(19) United States
(12) Patent Application Publication
(10) Pub. No.: US 2024/0182899 A1
(43) Pub. Date: Jun. 6, 2024

(54) SHORT MULTI-REPEAT RNA TARGETING GENE SILENCING

(71) Applicant: Kansas State University Research Foundation, Manhattan, KS (US)

(72) Inventors: David E. Cook, Manhattan, KS (US); Veerendra Sharma, Manhattan, KS (US); Sandeep Marla, Manhattan, KS (US); Geoffrey Morris, Fort Collins, CO (US)

(21) Appl. No.: 18/555,161

(22) PCT Filed: Apr. 14, 2022
(86) PCT No.: PCT/US2022/024863

§ 371 Date: Oct. 12, 2023

Related U.S. Application Data
(60) Provisional application No. 63/174,832, filed on Apr. 14, 2021.

(51) Int. Cl. C12N 15/113; C12N 15/82

(52) U.S. Cl. CPC C12N 15/113; C12N 15/8216; C12N 2310/11

(57) ABSTRACT

Short multi-repeat RNA targeting constructs for manipulating RNA targets and obtaining variable levels of gene silencing, and methods of using the same to silence RNA targets in mammals, insects, plants, and fungus. The constructs comprise two or more distinct guide nucleotide sequences (in the absence of CRISPR nuclease) that are complementary to one or more RNA targets, wherein each guide nucleotide sequence consists of a single-stranded antisense nucleotide fragment of 100 nt or less.

### CROSS-REFERENCE TO RELATED APPLICATIONS

The present application claims the priority benefit of U.S. Provisional Patent Application Ser. No. 63/174,832, filed Apr. 14, 2021, entitled GUIDE-INDUCED GENE SILENCING (GIGS), incorporated by reference in its entirety herein.

### FEDERALLY SPONSORED RESEARCH OR DEVELOPMENT

This invention was made with government support under grant no. D17AP00034 awarded by the Defense Advanced Research Projects Agency. The government has certain rights in the invention.

### SEQUENCE LISTING

The following application contains a sequence listing in computer readable format (CRF), submitted via EFS-Web as an ASCII text file entitled “Sequence_Listing,” created on Apr. 13, 2022, as 98,100 bytes, to serve as both the paper copy and CRF in compliance with 37 C.F.R. 1.821. The content of the ASCII text file is hereby incorporated by reference herein.

### BACKGROUND OF THE INVENTION

### Field of the Invention

The present invention relates to new RNA knockdown technology in which guide crRNA silences endogenous genes in the absence of the traditional CRISPR nuclease proteins, and offers a potential cis-genic solution for transcriptome engineering in plants and other organisms.

### Description of Related Art

New technological advances are needed to meet 21st century agricultural demands. The development of the clustered regularly interspaced short palindromic repeat (CRISPR) and associated proteins (Cas enzymes) for DNA editing holds significant promise to help increase agricultural productivity. However, targeting and editing DNA has limitations for multigene traits and cannot provide RNA-virus resistance. The editing of DNA may result in unintended and unpredictable pleiotropic phenotypes due to wide spatiotemporal gene function. Additionally, many target traits for improvement are polygenic in nature, and multi-gene genome editing will compound the problem of generating unpredictable and unwanted phenotypes.

CRISPR systems have been developed for various Cas proteins (Cas9, Cas12, Cas13) and have since been modified for RNA targeting, whereby the guide sequence directs the enzyme to edit a complementary RNA strand of the target. Cas13 (formerly C2C2) is a single effector recently identified in type VI CRISPR systems for RNA-guided RNA-interfering activity. See U.S. Patent Pub. No. 2019/035997, filed Jun. 19, 2017, and incorporated by reference herein in its entirety. Existing RNA interference (RNAi) technologies, which enable cleavage or inhibition of desired transcripts, have significant off-target effects and remain challenging for engineering targets.

### SUMMARY

As an alternative technology, we have developed a programmable RNA-guided targeting system based upon the CRISPR Cas guide platform, but which avoids the use of the CRISPR nuclease altogether, and the use of RNA sequences related to CRISPR. Thus, it does not require the introduction of “foreign” (i.e., bacterial) nucleic acids and therefore does not implicate the same issues as traditional transgenic technology. In general, the system involves nucleic acid constructs comprising short multiple repeat single stranded antisense sequences that are complementary to a target locus (either directly complementary or encode for the complementary antisense fragment). The guide sequences can be complementary to an endogenous target site of the host organism. Alternatively, the guide sequences can be complementary to an RNA target for an RNA virus that has infected the host organism. High levels of silencing of the target RNA are observed through the use of multiple repeat fragments of the guide sequence, leading to degradation of the target RNA and corresponding reduction of transcripts and thus downstream products. Unlike conventional CRISPR vectors, the construct does not include a second nucleotide sequence encoding for a CRISPR nuclease.

The platform is demonstrated initially using two Cas13a systems from Leptotrichia buccalis (Lbu) and Lachnospiraceae bacterium (Lba) as a platform for targeting trans, endogenous and viral RNA in plants. The function of the system was tested in Nicotiana benthamiana and Arabidopsis thaliana using biochemical and genomic techniques and the results show that both tested Cas13s can significantly reduce diverse target mRNAs. Unexpectedly, expressing the Cas13 CRISPR-RNA (crRNA) designed to express multiple guides provided target mRNA reduction independent of the Cas13 protein. Stable transgenic Arabidopsis plants have been developed to further understand this phenomenon, which has been further proved in soybean and extended to show efficacy using single-guide designed RNA related to the Cas9 system. Collectively, this research provides molecular characterization of new approaches for plant biotechnology, anti-viral resistance, and functional genomics.

The present disclosure concerns new techniques for gene silencing, in particular relying on guide RNA-alone to target RNA (instead of DNA) through target RNA degradation and without Cas enzymes traditional to CRISPR systems. The approach can be used, inter alia, to suppress plant viruses and plant endogenous transcripts as a new technique to reduce target RNA levels. The disclosure concerns expression constructs comprising single or multi-guide RNA segments or fragments. This technique was originally referred to as guide-induced gene silencing (GIGS), and relies on guide RNA sequences (or DNA sequence encoding for guide RNA) designed to be complementary to the target transcript. Guide-alone silencing can be induced using an array of anti-sense nucleotides that are separated by direct repeat loops, and other sequences, including short random nucleotide linkers. Moreover, multi-guide constructs can also include guides directly connected in the same expression cassette without intervening sequences.

Although the constructs may leverage endogenous RNAi machinery, these constructs are distinct from convention RNAi. Traditional small interfering RNA (siRNA) are usually induced by expressing a hairpin piece of RNA, typically ˜200 to 800 bp of a gene that has a linker, and then the same length of gene in the reverse complement. Sometimes the transcript is a short or small hairpin RNA (shRNA) that contains a sense and antisense sequence of 19-30 nucleotides intervened by a few (˜4) unpaired base pairs expressed as a single RNA molecule. This way, the one transcript has self-complementarity and folds on itself. For any design, a single transcript that has self-complementarity and forms the double-stranded RNA is introduced into the host organism or cell, and provides the signal to the endogenous machinery to initiate RNAi. Anti-sense RNA has also been used in which a transcript is expressed that is complementary to an mRNA fragment. This transcript binds to the mRNA to produce double-stranded RNA, and again, this triggers RNAi. However, antisense silencing using these approaches results in unpredictable silencing results, and is often unsuccessful unless it uses a very long piece of antisense RNA. The present technology leverages short (less than 100 nt) single-stranded, multiple repeat fragments or segments of a RNA guide sequence that is complementary to a target RNA. These short, multi-guide constructs unexpectedly demonstrate silencing of various targets in the absence of the CRISPR-Cas enzymes.

Advantages include the ability to apply precision biotechnology in crops without the use of “foreign DNA.” This approach, by eliminating components previously thought critical (e.g., Cas enzymes), further simplifies the ability to impact endogenous transcripts and knock down mRNA. The use of multiple short pieces of antisense RNA, which do not necessarily need to target contiguous regions of the target RNA, provide numerous advantages, including greater flexibility for design, greater quantitative range of target RNA reduction, and fidelity for silencing, resulting in fewer off-target effects (mRNA silencing of unintended targets). The platform is also superior to traditional RNAi in that constructs can be designed to more easily target multiple genes, which is fundamental for impacting complex traits or gene families. Moreover, the compact design of the expression cassettes makes them easier to use and clone.

Although exemplified in plant systems and crop improvement, the platform could be leveraged in the control of fungal pathogens (e.g., as a fungicide targeting genes in primary metabolism or cell wall components), as an herbicide (e.g., by selectively inhibiting required pathways in hard to control weeds), and as part of a rapid-response to emergent pathogens platform. The platform can be optimized to provide spatial and temporal silencing using SMRRTs that will be a key advancement in crop biotechnology. The platform can also be paired with existing and emerging delivery platforms to introduce the expression cassettes into the target host.

In one aspect, the disclosure concerns short multi-repeat RNA targeting constructs for manipulating RNA targets, said constructs comprising two or more distinct guide nucleotide sequences that are complementary to one or more RNA targets, wherein each guide nucleotide sequence consists of a single-stranded antisense nucleotide fragment of 100 nt or less, for example, 15 nt to 100 nt, for example, 80 nt or less, for example, 50 nt or less, for example, 22 nt to 30 nt. In any one of the disclosed embodiments, the guide nucleotide sequences are guide RNA (crRNA) or DNA encoding for the guide RNA, which are capable of binding or hybridizing to the RNA target. In any one of the disclosed embodiments, the RNA target is a coding sequence, preferably mRNA or pre-mRNA, or a non-coding sequence, preferably ncRNA, IncRNA, tRNA, or rRNA. In any one of the disclosed embodiments, the guide nucleotide sequences each bind to a different region of the same RNA target. In any one of the disclosed embodiments, the different regions are not contiguous loci of said RNA target. In any one of the disclosed embodiments, the different regions are contiguous loci on said RNA target. In any one of the disclosed embodiments, the guide nucleotide sequences each bind to a different RNA target In any one of the disclosed embodiments, the constructs can be comprised within one or more expression cassettes, which is preferably a single-stranded cassette. In any one of the disclosed embodiments, the constructs can be comprised within a single expression cassette. In any one of the disclosed embodiments, the two or more distinct guide nucleotide sequences are directly linked on said expression cassette. In any one of the disclosed embodiments, the two or more distinct guide nucleotide sequences are indirectly linked, such that each guide nucleotide sequence is separated by an intervening sequence on said expression cassette. In any one of the disclosed embodiments, the intervening sequences are selected from the group consisting of regulatory elements, direct repeats, and/or non-coding sequences of 30 nt or less.

In any one of the disclosed embodiments, the single expression cassette is a homogenous multimer wherein each of said two or more distinct guide nucleotide sequences consists of the same sequence and has complementarity with the same region of the same RNA target. In any one of the disclosed embodiments, the homogenous multimer comprises up to eight repeats of the same guide nucleotide sequence.

Alternatively, in any one of the disclosed embodiments, the single expression cassette is a heterogenous multimer wherein each of said two or more distinct guide nucleotide sequences consists of different sequences having complementarity with different RNA targets or different regions of the same RNA target.

In any one of the disclosed embodiments, the constructs are located on the expression cassette adjacent at least one regulatory element. In any one of the disclosed embodiments, the constructs are configured for co-expression. In any one of the disclosed embodiments, the constructs are free of nuclease-encoding nucleotide sequences. In any one of the disclosed embodiments, the constructs are free of nuclease protein.

The disclosure also concerns compositions for manipulation of RNA targets which comprise a plurality of short multi-repeat RNA targeting constructs according to any one of the embodiments or combinations of embodiments disclosed herein, dispersed in a carrier or vehicle.

The disclosure also concerns methods of modifying an RNA target, said method comprising delivering to said RNA target short multi-repeat RNA targeting constructs according to any one of the embodiments or combinations of embodiments disclosed herein. In any one of the disclosed embodiments, the method comprises introducing said short multi-repeat RNA targeting construct into a cell, tissue, organ, or organism. In any one of the disclosed embodiments, the cell is a eukaryotic cell. In any one of the disclosed embodiments, the cell is a mammalian, plant, insect, or fungal cell. In any one of the disclosed embodiments, the organism is a mammal, plant, insect, or fungus. In any one of the disclosed embodiments, the modification of the RNA target comprises reduced expression of one or more transcripts of the RNA target. In any one of the disclosed embodiments, the modification of the RNA target comprises degradation of one or more transcripts of the RNA target. In any one of the disclosed embodiments, modifying said RNA target comprises partially or completely silencing trans or endogenous gene expression associated with said RNA target. In any one of the disclosed embodiments, the modification of the RNA target comprises altered transcription or translation of at least one RNA product. In any one of the disclosed embodiments, the expression of the at least one product is decreased. In any one of the disclosed embodiments, the RNA target is an RNA virus. In any one of the disclosed embodiments, the RNA virus has infected a mammalian, plant, insect, or fungal cell, tissue, or organism. In any one of the disclosed embodiments, the method comprises delivering said constructs to said mammalian, plant, insect, or fungal cell, tissue, or organism to treat or prevent said infection.

The disclosure also concerns methods of gene silencing comprising delivering to a cell, tissue, organ, or organism, short multi-repeat RNA targeting constructs according to any one of the embodiments or combinations of embodiments disclosed herein, wherein at least one of said two or more distinct guide nucleotide sequences is complementary to an RNA target associated with said gene.

The disclosure also concerns methods of manipulating a plant characteristic, said method comprising delivering to a plant cell, tissue, or plant short multi-repeat RNA targeting constructs according to any one of the embodiments or combinations of embodiments disclosed herein, wherein at least one of said two or more distinct guide nucleotide sequences is complementary to an RNA target associated with a gene encoding for said plant characteristic. In one or more embodiments, the plant characteristic is vigorous growth, abundant foliage, longer primary roots, yield, height, and/or shoot water potential, pest resistance, drought tolerance, heat tolerance, salt tolerance, cold resistance, herbicide resistance, fungal resistance or reduced fungal susceptibility, viral resistance, or reduced viral susceptibility. In one or more embodiments, the plant is an invasive plant or weed, wherein said plant characteristic is altered to kill said plant or render said plant susceptible to herbicides.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a cartoon illustration of various SMRRT silencing constructs in accordance with embodiments of the invention, showing (A) homogenous multimers; and (B) heterogenous multimers.

FIG. 2 shows data demonstrating that Cas13 and GIGS reduce viral and endogenous target RNA in N. benthamiana. a, Schematic overview of the Cas13 transgene system. Guide crRNA responsible for RNA target specificity contain a single 28 nucleotide (nt) spacer antisense to the target RNA (single-guide, s-guide), multiple 28 nt spacers (multi-guide, m-guide), or lack the spacer (empty-guide). A diagram showing the genome of turnip mosaic virus (TuMV) expressing GFP and indicating the location the three targeting sites for the guide crRNA. b, The accumulation of GFP accumulation was assessed at 120 hours post inoculation based on GFP fluorescence. Areas of agroinfiltration are shown in dashed white circles. Individual treatments are labeled with numbers and shown schematically below the photographs. c, Nanostring RNA quantification for Cas13 and TuMV levels corresponding to labeled treatments for N. benthamiana spot infiltration. Samples expressed Cas13 (+) or not (−). d, Quantitative PCR for the endogenous transcript PDS following N. benthamiana leaf spot infiltration. e, Representative single leaf images of N. benthamiana following TRV systemic delivery of guide crRNA targeting the PDS transcript. Empty and non-target guides (NT-guide) did not cause photobleaching (white sectors), while the antisense and multi-guide (m-guide) did induce visible photobleaching.

FIG. 3 shows data demonstrating that Cas13 and GIGS function across plant species and are heritable. a, Representative images of tomato leaves following TRV systemic movement and photobleaching induced by GIGS (TRV_{m-guide}) and an antisense transcript (TRV_{asPDS}). TRV expressing a non-targeting guide crRNA (TRV_{NT-guide}) does not induce photobleaching. b, Measurements of chlorophyll content from SPAD meter readings for three independent plants. SPAD meter reading where taken from leaf sections showing photobleaching, and individual reading are shown as black points with the mean and standard deviation shown as a bar plot. c, qPCR measurement of the PDS transcript standardized to the EF1a transcript and relative to the NT-guide sample. Three independent samples were analyzed and individual data are shown as black points with the mean and standard deviation shown as bar plots. (d-i), Data for independent transgenic Arabidopsis lines. Data for plants expressing LbuCas13a are shown in dark blue and plants not expressing the protein are shown in light blue. Control lines expressing a hairpin construct against the TTG1 transcript are shown in grey. d, Trichome counts from the seventh leaf of T2 Arabidopsis lines. Ten plants were counted per independent line, listed below graph, with the individual counts shown as black points and the distribution represented as a violin plot. e, Leaf anthocyanin quantification from T₃ seedlings following sucrose treatment. Representative wells follow extraction shown below each bar plot. f, Representative plantlets following sucrose treatment showing anthocyanin pigmentation (i.e. purple color). g, Total flavonoids extracted from seeds collected from T₂ plants. Five independent seeds lots were analyzed per line, shown as black points. h, Seed proanthocyandin quantification from the same plants analyzed in (g). i, Quantification of the TTG1 transcript from three T₂ and three T₃ plants per line, individual data shown as black points. Statistical comparisons were made between the transformation control (no guide) and each treatment using a one-sided Mann-Whitney U-test with Benjamini-Hochberg (BH) multiple testing correction. Samples with p-values less than 0.05 (*), and 0.01 (**) are indicated.

FIG. 4 shows data demonstrating that GIGS is mediated by components of RNAi. (a-d), Uniquely mapped read counts to the PDS transcript collected from agroinfiltrated spots. The summary of read mapping is shown relative to the transcription start site (TSS) till the end of the predicted mRNA (2216 bp). Reads counts are shown as log 2 of counts per million+1 (CPM) averaged between the two replicates per treatment, which are listed above the plots. The position of the expressed single- and multi-guide crRNA are shown as vertical dashed line(s). The region spanning the hairpin construct is shown as a grey window. (e), Proportion of 21, 22, and 24 nt mapped sRNA to the PDS transcript averaged from two replicates. (f-l), similar to (a-e) but data collected from systemic leaves following TRV expression. (m), Representative images of leaves following TRV systemic delivery of multi-guide 1 (m-guide 1) targeting PDS, in addition to three variants of m-guide 1 that contained two base pair mismatches at each of the three guide crRNA. The mismatches were at positions 5,6 (mg 1[mm5,6]), positions 10,11 (mg 1[mm10,11]), and positions 21,22 (mg 1[21,22]). Controls include expressing TRV with a non-targeting guide (NT-guide) and TRV with a region of antisense sequence to PDS (asPDS). Photobleaching is seen as a loss of green color. (n), SPAD meter readings from photobleached leaf samples as described for (m). Data collected from a total of six independent leaves from two experiments. (o), Quantification of the PDS transcript using qPCR for the same samples as measured in (n). Data standardized to an endogenous transcript and normalized to the TRV expressing the NT-guide. (p), Representative images of leaves following TRV systemic delivery of single-guide 1 (s-guide 1) targeting PDS, and a Cas9 designed sgRNA designed to contain the same 28 bp targeting PDS as in s-guide 1. An sgRNA 1 control was expressed that contained the sequence in sgRNA 1, but with 50% mismatches to the PDS transcript (sgRNA 1[50% mm]). Control sgRNA were expressed with non-targeting guide sequence (NT-sgRNA). Photobleaching is seen as a loss of green color in the asPDS sample, while interveinal yellowing is visible in the samples expressing s-guide 1 and sgRNA 1. (q), SPAD meter readings from photobleached leaf samples as described for (p). Data collected from a total of six independent leaves from two experiments. (r), Quantification of the PDS transcript using qPCR for the same samples as measured in (q). Data standardized to an endogenous transcript and normalized to TRV expressing NT-sgRNA. Statistical comparisons were made between the NT-guide or NT-sgRNA and each treatment using a one-sided Mann-Whitney U-test with Benjamini-Hochberg (BH) multiple testing correction. Samples with p-values less than 0.05 (*), and 0.01 (**) are indicated. n.s., non-significant difference (p>0.05) as measured by Mann-Whitney U-test.

FIG. 5 shows data demonstrating that Cas13a mediated efficient virus interference. (a) Images of N. benthamiana leaves from 48 hours post inoculation (hpi) to 120 hpi shown under UV light to visualize GFP fluorescence. The Cas13 protein (either LbuCas13 or LbaCas13) were expressed with a guide and the TuMV expressing GFP virus as indicated by the schematic diagram and numbering. The areas of agro-infiltration are indicated with white dashed circles. Higher GFP signal results from increased virus accumulation (b) Quantification of viral genome accumulation using qPCR. TuMV levels were standardized to a plant endogenous transcript and three samples were collected per time point. c,d, GFP protein accumulation over the time series of inoculated leaves expressing LbuCas13a (c) or LbaCas13a (d). The lanes are labeled above the images for each time point, while the anti-GFP (α-GFP) panel shows signal from western blot, and the commassie brilliant blue (CBB) stained gel panel shows the loading control.

FIG. 6 shows data demonstrating that CRISPR inhibits TuMV with and without the Cas13 protein. a, Quantification of TuMV accumulation from N. benthamiana transient spot expression. Leaves were inoculated with TuMV, and a combination of Cas13 and guide crRNA as indicated to the left of the barplot. Individual samples are shown as black dots and the mean is shown as a bar with standard error. TuMV levels were standardized to the plant endogenous EF1α transcript and normalized to the Cas13 alone sample. Five samples were collected for each treatment. b, Nanostring quantification for using four different probes: TuMV (Coat protein, CP), and three endogenous controls from N. benthamiana (EF1α, PP2aa2, RPL23a). Samples expressed either the LbuCas13a protein (−) or no transgene (−), along with no-guide (−), a single-guide (s-guide), or a multi-guide crRNA (m-guide). Three independent samples were analyzed per treatment.

FIG. 7 shows data demonstrating that GIGS can function systemically to achieve virus interference. a, Schematic diagram of the TRV expressing guide crRNAs and TuMV expressing GFP. The two infectious clones are mixed and agroinfiltrated into N. benthamiana. At 7 days post inoculation, plants are visualized under a GFP lamp. TRV expressing an empty or NT-guide does not target TuMV and GFP accumulates in upper leaves. TRV expressing crRNA targeting TuMV results in GIGS and they fail to accumulate GFP. b, Representative images of N. benthamiana plants under UV light at 7 days post inoculation. The systemic movement of TuMV is evident based on the accumulation of GFP flourescence. The single-guide 2 (s-guide 2) and multi-guide (m-guide) stopped systemic TuMV infection. c, Upper leaves were checked for GFP accumulation by western blot corresponding to images shown in (b). The GFP antibody panel (αGFP) shows GFP signal while commassie brilliant blue panel (CBB) shows protein loading. d, The TuMV genome was quantified from three independent plants (shown as black points) using qPCR. The mean and standard deviation are shown as barplots. Samples were standardized to the EF1α endogenous transcript and normalized to the empty-guide control levels.

FIG. 8 provides data showing guide crRNA design and target sites for endogenous mRNA reduction by GIGS. Schematic of single- and multi-guide crRNA (s-guide and m-guide, respectively), along with control non-targeting guides (NT-guide) and RNAi inducing Hairpin. The approximate location where the respective guides are antisense to the PDS transcript (i.e. their target location) are denoted by filled arrows. The region covered by the hairpin construct is shown as a grey bar.

FIG. 9 provides data showing endogenous mRNA reduction mediated by Cas13-dependent and GIGS expression. a, Reduction in PDS mRNA accumulation as measured by northern blot analysis using a PDS probe (PDS panel). Samples expressing the Cas13a transcript are indicated (Cas13+) and correspond to signal from the Cas13 probe (LbuCas13 panel). Samples expressed no guide (−), a non-target guide (NT), one of three single-guides (1,2, or 3), a multi-guide (M), or a hairpin construct (H). The presence of roughly equal RNA amounts was confirmed by the abundance of ribosomal RNA signal (RNA loading panel). b, Quantification directly on RNA samples using nanostring for Cas13 (Cas13 probe) and phytoene desaturase (PDS probe) mRNA. The presence (+) or absence (−) of Cas13 expression is indicated to the left, along with the expression of a single-guide (s-guide) or the multi-guide (m-guide).

FIG. 10 is an illustration of the experimental design for systemic endogenous mRNA reduction by GIGS. Schematic of the TRV expression vector system (RNA1 and 2). RNA2 was engineered to contain the pea early browning virus (PEBV) promoter to express single-, multi-, and NT-guides. An antisense fragment (371 bp) to PDS was also inserted into the RNA2 cloning sight to induce RNAi against PDS. Infectious clones are agroinfiltrated. TRV moves systemically in the plant, delivering the respective guide crRNA. Those that cause PDS mRNA silencing result in a bleaching phenotype (i.e. white sectors) seen on upper leaves.

FIG. 11 provides data showing systemic endogenous mRNA reduction by GIGS. a, Representative images of N. benthamiana plants two weeks post TRV inoculation. TRV moved systemically to the top portion of the plant and expressed guide crRNAs targeting PDS or controls. Photobleaching caused by PDS mRNA reduction is visible as white or yellow sectors in the upper leaves. Each image is labeled with the guide delivered by TRV. b, SPAD meter readings from photobleached areas of leaves. One reading was taken per infiltrated plant. Three independent plants were infiltrated. c, The PDS transcript was quantified using qPCR from three independent leaves. Data from each sample is shown as a black point with the mean and standard deviation shown as a boxplot. Samples were standardized to the EF1α endogenous transcript and normalized to the empty-guide control levels.

FIG. 12 shows data demonstrating that Cas13-dependent and GIGS T1 transformed A. thaliana lines and display phenotypes consistent with TTG1 reduction. a, Leaf trichome counts from individual T1 lines shown as black points and the distribution shown as a violin plot. Three single-guides (g1, g2, g3) or a multi-guide (Mg) were transformed into plants with Cas13a (dark blue) or without (light blue). Plants were transformed with a hairpin construct (grey) designed to silence the TTG1 transcript. Statistical comparisons to the non-transformed control (WT, wild-type) were made as one-sided Mann-Whitney U-test with Benjamini-Hochberg (BH) multiple testing correction. Samples with p-values less than 0.05 (*) are indicated. b, Five lines from each transformation group were assessed for TTG1 transcript levels using qPCR. The values were standardized to AtEF1α endogenous control and normalized to the WT control. Individual data points are shown as black points and the mean and standard deviation shown as a barplot. c, Individual T1 plants were self fertilized, and five lots of seed from each plant (technical replicates) were analyzed for seed total flavonoid content. Transformants expressing Cas13a are shown in dark blue while those not expressing Cas13a are shown in light blue. Individual data points are shown as black points and the mean and standard deviation shown as barplots: WT, wild-type; C13, Cas13a expressing; empty, no Cas13a protein; Hairpin, expressing a 197 bp hairpin against TTG1. Numbers indicate the line numbers for the indicated treatments shown below.

FIG. 13 provides data showing expression and translation products of Cas13 transgenic Arabidopsis. a, The Cas13 transcript is only detected using reverse-transcription PCR (RT-PCR) in lines transformed with the LbuCas13a transgene (Lbu, top panel). The endogenous transcript coding for Elongation factor 1 alpha (EF1α) is shown as a control. The western blot panel using an anti-HA antibody (α-HA) detected a single band corresponding to the size of the 3×HA-LbuCas13a protein (145 kDa). Ponceau strain was used a protein loading control. b, Protein was isolated from the same lines used in (a) and re-analyzed by western blot. Higher exposure time detected a single band corresponding to 3×HA-LbuCas13a in line #1 as well as line #7. The results indicate likely differences in protein translation or possibly stability between the different transgenic lines.

FIG. 14 provides data showing guide crRNA design for multi-guide 1 and three corresponding multi-guides with base pair mismatches. Schematic of multi-guide 1 (m-guide 1) expressed as a single RNA transcript from the U6 promoter. The multi-guide crRNA targets the PDS transcript at three locations, shown as three filled arrows indicating the relative position on the PDS transcript. For each of the 28 nt guide targeting sequences (crRNA 1, 2, and 3), the complementary sequence is shown (multi-g1), the guide with mismatches at position 5,6 (multi-g1[mm5,6]) (highlighted in green), the guide with mismatches at position 10,11 (multi-g1[mm10,11]) (highlighted in blue), and the guide with mismatches at position 21,22 (multi-g1[mm21,22]) (highlighted in purple). *, indicates where the four guide crRNA sequence alignments are identical.

FIG. 15 shows data demonstrating that guide crRNA with mismatches at base pairs 10,11 do not elicit GIGS. Representative images of plants following Tobacco Rattle Virus (TRV) systemic movement. Plants infected with TRV expressing a non-targeting guide crRNA (NT-guide) have a normal green leaf appearance. Plants infected with TRV containing an antisense phytoene desaturase (asPDS) fragment display photobleaching in upper leaves following TRV systemic movement and the triggering of RNAi PDS silencing. TRV expressing multi-guide 1 (m-guide 1), multi-guide 1 with mismatches between the target and guide at positions 5 and 6 (m-guide 1[mm 5,6]), or multi-guide 1 with mismatches at positions 21 and 22 (m-guide 1[mm 21,22]) also display photobleaching in upper leaves. Plants inoculated with TRV expressing multi-guide 1 with mismatches between the target and guide at positions 10 and 11 (m-guide 1[mm 10,11]) have the same normal green appearance as the NT-guide samples.

FIG. 16 shows data demonstrating that Cas9 sgRNA can elicit GIGS photobleaching in N. benthamiana. a, Schematic of guide designs targeting PDS transcript for Cas9 (single guide RNA, sgRNA) and Cas13 (single-guide, s-guide). Each guide contains 28 nt antisense to the PDS transcript (PDS spacer, grey window) at the 5′ end. The Cas9 sgRNA control contained 50% mismatch sequence to the PDS transcript (sgRNA 1 [50% mm]). Mismatch nucleotides highlighted in red, while shared nucleotides are designated with an (*) under the alignment. The 3′ end of the Cas9 crRNA contains the 78 base pair trans-activating crRNA (tracrRNA, green). The Cas13 crRNA contains the 37 bp direct repeat (DR, beige). Total length for each crRNA is shown to the right of each crRNA. b, Representative whole plant images showing GIGS induced photobleaching. Plants expressing the control non-targeting single guide RNA (NT-sgRNA) from TRV do not display any abnormal leaf color. Strong photobleaching is seen from plants expressing TRV containing an antisense PDS fragment (asPDS). Clear phenotypic differences in leaf greenness are seen when TRV expressed Cas13 (s-guide 1) or Cas9 (sgRNA 1) designed crRNA against PDS. These plants show interveinal yellowing. The control Cas9 crRNA with 50% mismatches to the PDS transcript (sgRNA 1[50% mm]) does not display any visible alteration in leaf greenness.

FIG. 17 shows data demonstrating that PDS multi-guide crRNA expressed with three promoters produced strong GIGS. a, Schematic of PDS guide crRNA targeting locations on PDS transcript. PDS multi-guide crRNA was expressed as a single RNA molecule using a single PEBV promotor, and three separate s-guides using three PEBV promotors. b, Two-component Tobacco rattle virus (TRV) system for systemically expressing crRNA through agro-mediated TRV infection, and evaluating targeted gene silencing phenotype after systemic TRV infection. c, Representative images of a lower leaf from N. benthamiana plants after two-weeks of TRV systemic delivery with PDS m3g3 and three s-guides. d, Chlorophyll content measured with SPAD meter two-weeks after systemic delivery of crRNA. e, Relative PDS expression, quantified using qRT-PCR, compared between NT-guide and each treatment. Statistical comparisons were made using one-way ANOVA in R, multiple comparisons of treatments by means of Tukey were performed using HSD.test function from the agricolae R package. Treatments with the same letter are not significantly different.

FIG. 18 shows data evaluating minimum multi-guide crRNA length required for GIGS. a, Schematic representation of PDS guide crRNA and antisense PDS construct targeting locations on PDS transcript. Schematic of PDS m3g3.28nt crRNA with 37 bp LbuCas13a direct repeat (LbuDR) and three 28 bp crRNA. PDS m3g3 with varying lengths were generated by reduced crRNA length from the 3′ end of each crRNA. For example, PDS m3g3.20nt was generated by removing 8 nt from the 3′ end of each crRNA. b, Representative images of a lower leaf from N. benthamiana plants after two-weeks of systemic TRV delivery of transformation control (TRV_{empty}) and reduced guides of varying lengths (28-18 nt). c, Chlorophyll content or leaf greenness measured with SPAD meter two-weeks after systemic delivery of crRNA. d, Relative PDS levels quantified using qPCR. Statistical comparisons were made using one-way ANOVA in R, multiple comparisons of treatments by means of Tukey were performed using HSD.test function from the agricolae R package. Treatments with the same letter are not significantly different.

FIG. 19 shows data demonstrating that multi-guide crRNA design does not require a direct repeat. a, Schematic representation of PDS m3g3 crRNA with LbuCas13a direct repeat (LbuDR) was replaced with Arabidopsis miR170 stem-loop (stem-loop, 24 nt), without four-base-pair stem (loop, 16 nt), a random sequence of 20 nt (random), and without DR (noDR). b, Representative images of a lower leaf from N. benthamiana plants after two-weeks of TRV systemic delivery with TRV_{empty}, PDS m3g3 and NT m3g3 stem-loop, loop, random, and noDR crRNA constructs. c, Chlorophyll content or leaf greenness measured with SPAD meter two-weeks after systemic delivery of crRNA. d, Relative PDS expression, quantified using qRT-PCR, compared between the transformation control (TRV_{empty}) and each treatment. Statistical comparisons were made using one-way ANOVA in R, multiple comparisons of treatments by means of Tukey were performed using HSD.test function from the agricolae R package. Treatments with the same letter are not significantly different.

FIG. 20 shows data demonstrating that altering guide order in a PDS multi-guide did not impact visual photobleaching. a, Schematic of guide order crRNA constructs generated for determining the effect of guide order on GIGS. b, Representative images of a lower leaf from N. benthamiana plants after two-weeks of TRV systemic movement of TRV_{empty}, NT m3g3, and PDS m3g3 constructs with rearranged guide order. c, Chlorophyll content or leaf greenness measured with SPAD meter two-weeks after systemic delivery of crRNA.

FIG. 21 shows data demonstrating that increasing PDS multi-guide crRNA number affected GIGS strength. a, Schematic of PDS m3g3 and m6g6 crRNA, and antisense PDS construct locations on PDS transcript. b, Representative images of a lower leaf from N. benthamiana plants after two-weeks of TRV systemic movement with m3g3 and m6g6 crRNA with or without direct repeat (LbuDR and noDR). c, Chlorophyll content measured with SPAD meter two-weeks after systemic delivery of crRNA. Statistical comparisons were made using one-way ANOVA in R, multiple comparisons of treatments by means of Tukey were performed using HSD.test function from the agricolae R package. Treatments with the same letter are not significantly different.

FIG. 22 shows data demonstrating that increased guide dosage elicited an increase in visible photobleaching phenotype. a, Schematic of PDS s-guides, m3g1, and m6g1 crRNA for silencing PDS expression. b, Representative images of a lower leaf from N. benthamiana plants after two-weeks of TRV systemic movement with three s-guides, three m3g1, and three m6g1 crRNA for targeting PDS expression. c, Chlorophyll content measured with SPAD meter two-weeks after systemic delivery of crRNA. Statistical comparisons were made using one-way ANOVA in R, multiple comparisons of treatments by means of Tukey were performed using HSD.test function from the agricolae R package. Treatments with the same letter are not significantly different.

FIG. 23 shows images of plants showing systemic TRV movement of reduced guide length PDS m3g3 crRNA.

FIG. 24 shows images of plants demonstrating that increasing the number of crRNA in a PDS multi-guide elicited a strong photobleaching phenotype.

FIG. 25 shows images of plants demonstrating that the strength of GIGS elicited by increased guide dosage is RNA target dependent and that increased guide dosage resulted in stronger photobleaching.

FIG. 26(A) shows images of soybean hairy roots treated with the SMRRT silencing constructs from Example 3.

FIG. 26(B) shows images of soybean hairy roots treated with the SMRRT silencing constructs, from Example 3.

### DETAILED DESCRIPTION

The present invention is concerned with new oligonucleotide constructs for manipulation of RNA targets, and their downstream products, e.g., for modification of endogenous or transgenic cellular dynamics, genotype-phenotype, and/or protein expression, as well as for targeting RNA viruses and fungi for pathogen inhibition, and engineered antiviral or antifungal activity and immunity. These new constructs are derivatives of conventional CRISPR-Cas RNA editing platforms, but function in the absence of any Cas nuclease protein. Moreover, silencing appears to be mediated by RNA endonuclease reduction and not translational inhibition of target mRNA. Further, the level of silencing can be titrated or adjusted based upon the dosage (e.g., number) of the constructs delivered to the cells, including the number of guide sequences expressed that are antisense to the target RNA. Thus, an advantage of the construct design is that it is not a binary “on” or “off” silencing approach, but rather since it targets transcript levels, the level of reduced transcripts and/or silencing of the downstream gene products can be controlled based upon the dosage of guide sequences delivered, to only partially reduce the target, and further that gradations or gradients of reduction may be achieved from significant or near complete reduction of target transcripts to minor or minimal reduction of target transcripts (and reduction levels in between, e.g., moderate reduction). Thus, the reduction of target transcript (and thus amount of silencing) is adjustable or variable along a gradient, e.g., from about 1% reduction up to near 100% reduction, and any integer between 1 and 100% reduction of the target transcript, preferably about 10%, 20%, 30%, 40%, 50%, 60%, 70%, 80%, or 90% reduction. Moreover, through the use of tissue specific promoters in the constructs, the targeting can be even more precise to selectively suppress the gene targets (not only selectively suppress the levels along a gradient, but also suppress only in certain tissues).

The constructs generally comprise short multiple repeat fragments (SMRFs) that target one or more loci of interest for silencing via targeting of complementary RNA, thus giving rise to short multi-repeat RNA targeting (SMRRT) gene silencing. The constructs comprise or encode for single-stranded, short antisense nucleotide fragments that are complementary to a target RNA sequence (e.g., in an mRNA), such that the corresponding transcripts (and their downstream products) will be inhibited or reduced. Thus, embodiments described herein include methods and compositions for modulating (e.g., reducing) gene, transcripts, and/or protein expression in cells. The term gene “expression” refers to any stage in the process of converting genetic information encoded in a DNA sequence or gene into RNA (e.g., mRNA, IRNA, tRNA, or snRNA) through transcription of the DNA sequence or gene (i.e., via the enzymatic action of an RNA polymerase), and into protein, through translation of mRNA. DNA sequence or gene expression can be regulated at many stages in the process.

The SMRFs are derived from CRISPR Cas guides and typically comprise, consist essentially, or consist of a CRISPR-type guide RNA (crRNA) sequence (or nucleotide sequence encoding for the crRNA sequence), which is capable of binding or hybridizing to the target RNA.

In the context of formation of a conventional CRISPR RNA-targeting complex, the target RNA sequence refers to a sequence to which a guide is designed to have sufficient complementarity, such that the guide will hybridize with the target and promote formation and binding of the CRISPR complex and interaction of the Cas nuclease with the target sequence. The target RNA sequence may be located in the nucleus or cytoplasm of a cell. The target RNA sequence may be a coding (e.g., mRNA or pre-mRNA) sequence or a non-coding (e.g., ncRNA, IncRNA, tRNA, rRNA) sequence.

Various guides are known for the different CRISPR platforms and/or can be determined manually or using a number of online tools or software programs. For example, target RNA can be selected by based on the target trait of interest such as from published experiments, unpublished experiments linking RNA expression and resulting protein function to a trait of interest, sequences present in a viral or fungal pathogen of interest, or other endogenously transcribed RNA in an organism the user wishes to target, taking into account the conserved nature of the target as well as any structural considerations for secondary structures of the target transcripts. From this information, guide sequences can be designed (and then synthetized) as respective antisense sequences to the target RNA, specifically with complementarity to one or more positions, locations, or regions along the target RNA sequence, which can be contiguous target sequences or non-contiguous target regions in the target RNA. The SMRFs can target any region from the 5′ start to the 3′ end of the target RNA, including sequences that do or do not contain protein translation sequences. Target regions can be further refined by modeling target RNA secondary structure, which could be based on commercially available software and online tools such as, but not limited to, RNAfold (rna.tbi.univie.ac.at//cgi-bin/RNAWebSuite/RNAfold.cgi). Regions with less secondary structure interpreted through less self-pairing and higher disorder or entropy are considered as better regions for targeting with guides. Target regions can be further refined by modeling RNA-RNA interactions, which could be based on commercially available software and online tools such as, but not limited to, RNAup (rna.tbi.univie.ac.at//cgi-bin/RNAWebSuite/RNAfold.cgi). Here, potential or candidate antisense sequences are checked against the target sequence to model the energy of guide-target pairing and energy to open the target sequence. Better guides have a more negative free energy of binding, that is they are energetically more favorable, and require a lower opening energy for the target sequence. Preferably, off target effects are reduced in the system, such that the desired degree of complementarity between a guide sequence and its corresponding target sequence is greater than 95%, more preferably greater than 98%, more preferably greater than 99%, and even about 100% (+/−0.5%).

In the present disclosure, the construct is free of CRISPR-associated (Cas) nucleases or proteins (or nucleic acids encoding such nucleases or proteins), and any other sequence derived from the CRISPR-Cas system. Thus, while the conventional nomenclature of “guide” or crRNA sequence is used herein, it will be appreciated that the targeting constructs used herein no longer “guide” a CRISPR enzyme to the target, and no longer contain characteristic bacterial or archaea derived sequences. Nevertheless, the technology can leverage the existing libraries of CRISPR RNA guides which have been identified for various systems and conditions, and can be used in the constructs described herein to target the desired RNA.

This technology is currently demonstrated in four different plant systems using numerous multi-guide constructs, but can be applied in various eukaryotic systems including animals, insects, fungi, and plants, as well as for inhibition of RNA viruses. In one or more embodiments, each guide segment or fragment consists of a sequence length of 15 nucleotides (nt) up to 100 nt, preferably from 20 nt to 80 nt, and preferably from 20 nt to 50 nt, more preferably from 22 nt to 30 nt. Notably, the guide construct does not need to target a contiguous piece of target RNA.

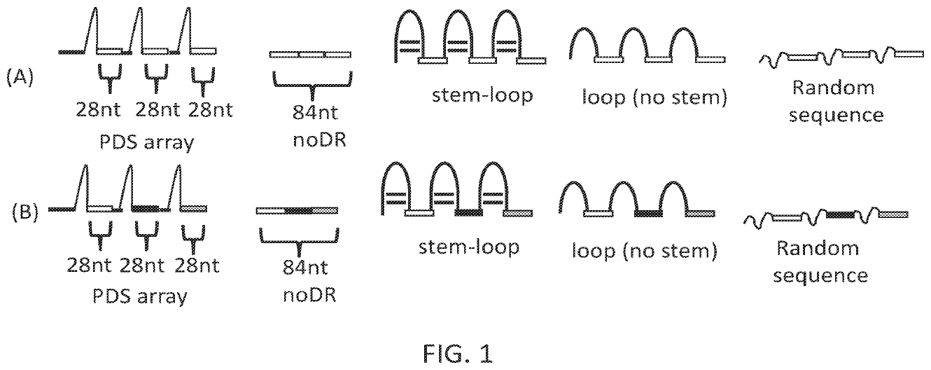

The architecture of the SMRFs for SMRRT silencing can be arranged in numerous ways as illustrated in FIGS. 1(A) and 1(B), subject to certain design constraints, namely that the constructs comprise two or more guide fragments targeting one or more RNA targets (i.e., short anti-sense fragments complementary to a region of one or more RNA targets). As illustrated in FIG. 1(A), in one or more embodiments, the SMRF construct can comprise a homogeneous multimer comprising two or more copies of the same guide sequence on the same construct (expression cassette). In such homogenous multimers, the two or more guide sequences can be directly linked (e.g., immediately adjacent on the cassette without any intervening sequences or nt). In such homogenous multimers, the two or more guide sequences can be indirectly linked, such that each guide fragment is separated by an intervening sequence. In the homogenous multimer, the individual guide fragment repeats, each have complementarity with the same region of the same RNA target. In the homogenous multimer, the individual guide fragment repeats could also have complementarity with similar (homologous) regions of different RNA targets. In one or more embodiments, as illustrated in FIG. 1(B), the SMRF construct can comprise a heterogenous multimer comprising two or more different guide sequences on the same construct (expression cassette). In such heterogenous multimers, the two or more different guide sequences can be directly linked (e.g., without any intervening sequences or nts). In such heterogenous multimers, the two or more different guide sequences can be indirectly linked, such that each guide fragment is separated by an intervening sequence. In the heterogenous multimer, the different guide fragments can each target the same RNA target, but have complementarity with a different region of the target. In one or more embodiments, the different guide fragments target loci that are spaced apart from one another on the target RNA (e.g., opposite ends, or beginning and middle, or middle and end of the target region, etc.). In one or more embodiments, the different guide fragments target contiguous loci on the target RNA (e.g., different sequentially located sequence segments). In the heterogenous multimer, the different guide fragments can each target a different RNA target entirely (e.g., a first guide fragment with complementarity to a first RNA target and a second guide fragment with complementarity to a second RNA target that is a different RNA molecule from the first RNA target).

The intervening sequences, if present, in the homogenous or heterogenous multimers can comprise (consist essentially or even consist of) direct repeat sequences (typically included with CRISPR guides), as well as random short intervening sequences (e.g., less than 30 nts). The direct repeat sequences can comprise a stem loop or other secondary structures (e.g., loop only, lacking the stem). In one or more embodiments, the intervening sequences, if present, are each located upstream (i.e., 5′) from each respective guide fragment. In one or more embodiments, the intervening sequences, if present, are each located downstream (i.e., 3′) from each respective guide fragment. With the exception of small loops or stem loops, the guide fragments are not designed to form any other secondary structures and are free of hairpin loops-they are not self-complementary and do not form double-stranded structures with themselves. Each fragment in the multimer can be driven by the same single promoter 5′ to the entire expression cassette or respective promoters 5′ to each fragment. Thus, intervening sequences in the multimers can include promoters or other regulatory sequences between the sequences.

In one or more embodiments, the present disclosure contemplates expression cassettes comprising 2 or more guide fragments directly or indirectly connected. In one or more embodiments, the present disclosure contemplates expression cassettes comprising 3 or more guide fragments directly or indirectly connected. In one or more embodiments, the present disclosure contemplates expression cassettes comprising 4 or more guide fragments directly or indirectly connected. In one or more embodiments, the present disclosure contemplates expression cassettes comprising 5 or more guide fragments directly or indirectly connected. In one or more embodiments, the present disclosure contemplates expression cassettes comprising 6 or more guide fragments directly or indirectly connected. In one or more embodiments, the present disclosure contemplates expression cassettes comprising 7 or more guide fragments directly or indirectly connected. In one or more embodiments, the present disclosure contemplates expression cassettes comprising 8 or more guide fragments directly or indirectly connected. In such embodiments, the multiple guide fragments are expressed as a single transcript (same sequence on a single transcript) for SMRRT silencing.

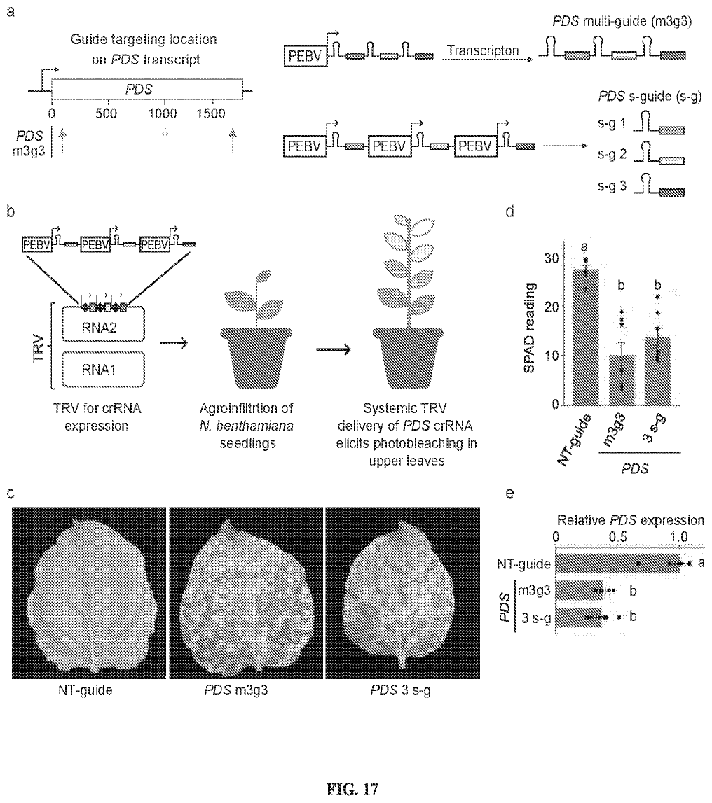

In one or more embodiments, compositions are contemplated herein which comprise multiple guide fragments, each expressed as multiple independent transcripts of their respective expression cassettes for SMRRT silencing (see FIG. 17a). Such single-guide constructs may comprise respective promoter sequences, as well as direct repeats, stem loops or other secondary structures. These single-guide constructs are co-expressed in the system.

Thus, embodiments described herein contemplate inhibition or reduced expression of target RNA transcripts and/or proteins via delivery the SMRFs, which may be located on the same vector or co-expressed on different vectors of the system. In one or more embodiments, the guide fragments are designed and combined in the expression cassette(s) so as to target multiple regions of a target RNA. In one or more embodiments, the guide fragments are designed and combined in the expression cassette(s) so as to target multiple RNA targets. The SMRF constructs can comprise DNA that encodes for the guide fragments (and is transcribed in vitro or in vivo into the corresponding RNA). The SMRF constructs can comprise transcribed crRNA. The SMRF constructs can comprise chemically synthesized RNA. The SMRF constructs can be further modified to improve stability (e.g., with 5′ or 3′ capping), addition of a detectable label (e.g., fluorescent moiety) and the like. For ease of reference herein, the terms crRNA or guide fragments are used interchangeably and may be referred to as “RNA guides” inclusive of either the RNA molecule itself or a DNA molecule which encodes for the RNA guide fragment. Thus, although the DNA sequence may be depicted, it is conventional in the art to still refer to this construct as an “RNA” guide or “crRNA guide.”

Embodiments described herein encompasses methods for delivering multiple guide fragments, wherein each guide fragment is specific for a different target locus of interest thereby modifying multiple target loci of interest. Alternatively, multiple repeats of the same fragment can be delivered, which appears to provide the best balance of robust silencing with fewer off-target effects. As demonstrated in the data, a stronger reduction in target mRNA can be observed using multi-guide cassettes. The approaches described herein can be applied to improve emerging as well as existing CRISPR-based gene editing systems, and SMRRT silencing approach can be used with new CRISPR-Cas guides as they are developed or identified.

In one or more embodiments, described herein are methods of modifying a target locus of interest, in particular in eukaryotic or prokaryotic cells, tissues, organs, or organisms, in particular in animal, plant, bacterial, or fungal cells, tissues, organs, or organisms. In one or more embodiments, described herein are methods of reducing the expression of one or more transcripts of a target locus in an organism. In one or more embodiments, described herein are methods of reducing the expression of one or more genes “associated with” the RNA target (e.g., genes encoding for the RNA target that is ultimately translated into the gene product, typically having a detectable phenotype). In other words, the target RNA transcripts are associated with the target gene whose expression is desired to be altered. Degradation of the target RNA transcripts prevent translation of the gene product, which can be detected as reduced protein expression and/or altered phenotype.

In one or more embodiments, the SMRF constructs can be used in methods for partially or completely silencing trans or endogenous genes in plants. As such, this technology offers a potential solution to bypass antagonistic pleiotropy by silencing targeted genes in a tissue-, age-, or inducible-specific manner. The construct or vector can be introduced by any suitable method, including, without limitation, agroinfiltration, biolistic particle delivery system, microprojectile bombardment, viral infection, Agrobacterium-mediated transformation (Agrobacterium tumefaciens), PEG- or electroporation-mediated uptake, particle bombardment-mediated delivery, microinjection, nanoparticle delivery, or liposomal delivery, to produce modified plant cells, tissues, or plants. The term “bombardment” with respect to transformation refers to the process of accelerating particles towards a target biological sample (e.g., cell, tissue, seedling, etc.) to effect wounding of the cell membrane of a cell in the target biological sample and/or entry of the particles into the target biological sample. In some embodiments, the constructs can be formulated in delivery vehicles that facilitate uptake of the constructs through the root system, leaves, or other tissues of the plants. The constructs may be applied to the leaves or other tissue of the plants. The constructs may be applied to the roots or in the vicinity of the roots (e.g., to the growth medium, soil, sand, dirt, etc.) in which the plant is rooted. The constructs can also be delivered to the meristematic tissue of developing or developed plants.

In any case, introduction of the constructs results in altered levels of expression of the target in the plant. References to altered “levels” of expression refers to the production of gene product(s) in modified plants in amounts or proportions that differ from that of normal, control, or non-modified plants. The modifications can result in plants having favorable or improved morphological or phenotypical characteristics, such as: vigorous growth, abundant foliage, longer primary roots, yield, height, and/or shoot water potential, pest resistance, drought tolerance, heat tolerance, salt tolerance, cold resistance, herbicide resistance, fungal resistance or reduced fungal susceptibility (e.g., by targeting RNA transcripts of a susceptibility gene), viral resistance, or reduced viral susceptibility (e.g., by targeting RNA transcripts of a susceptibility gene), water-use efficiency, nutrient uptake, or altered chemical profiles such as in the leaf, shoot, stem, seed, flower, tuber, hypocotyl, root, or lateral root. Altered chemical profiles could include changes in the relative amount of a chemical, or could include changes in the structure of the chemical, or could include changes in the spatial partitioning of the chemical compared to a normal, or control, or non-modified plants. Alternatively, the constructs can be used as herbicides to result in plants having impaired morphological or phenotypical characteristics, such as heat, drought, salt susceptibility, shortened root systems, photosensitivity, and the like. The constructs can be used to target critical pathways, such as photosynthesis, or derived photosynthetic carbon utilization or movement, to cause death of unwanted plants, or to render the plant susceptible to herbicides.

The methods can be used for monocotyledonous as well as dicotyledonous plants. Examples of suitable plants include, without limitation, wheat (Triticum sp.), barley (Hordeum sp.), rice (Oryza sp.), maize (Zea sp.), rye (Secale sp.), corn (maize), cassava, sorghum (Sorghum bicolor), and cereal crops (grasses in monocot family Poaceae including annual and perennial grasses), as well as soybeans (Glycine sp.), tomato (Solanum sp.), cotton (Gossypium sp.), tobacco (Nicotiana tabacum), potato (Solanum tuberosum), strawberry (Fragaria spp.), olive trees, grape vines, other embodiments of fruit and nut trees, poplar trees, as well as research plants such as Arabidopsis, Brachapodium spp., Nicotiana benthamiana, as well as photosynthetic algae. The methods can also be used for weeds or other invasive plant species, such as common waterhemp, palmer amaranth, giant/common ragweed, common lambsquarters, velvetleaf, giant foxtail, woolly cupgrass, burcucumber, morning glory, horseweed, marestail, sunflower, bluegrass, canada thistle, foxtail spp., johnsongrass, cheat grass, crabgrass, nutsedge, purple deadnettle, kochia, nightshade, pigweed, and kudzu vine.

The methods can be used to establish stable trans- or cis-genic plants expressing guide RNA constructs against an RNA target. In one or more embodiments, the SMRRT silencing constructs are inheritable, giving rise to inheritable phenotypes in progeny. Thus, embodiments herein contemplate seeds, seedlings, and progeny of modified plants, wherein the SMRRT silencing constructs are expressed in the seeds, seedlings, or progeny. Further, embodiments herein contemplate progeny with multiple traits targeted for modification, for example by using traditional breeding techniques to cross two or more plants that have been modified using the SMRRT silencing constructs, thus yielding seeds, seedlings, or progeny, expressing both of the inherited guide RNA constructs. It will be appreciated that these stably incorporated constructs can thus be used to achieve any combination of modified characteristics or traits in plants.

In one or more embodiments, the SMRF constructs can be used in methods for partially or completely silencing endogenous genes in fungi or reducing target RNA levels, such as components of cell wall synthesis including but not limited to glucan synthases, synthesis of membrane components such as ergosterol, components of primary metabolism or other components of primary growth.

In one or more embodiments, the SMRF constructs can be used in methods for partially or completely silencing endogenous genes in bacteria or reducing target RNA levels, such as by targeting bacterial small RNA or other RNA transcripts in critical pathways for bacterial growth and/or metabolism. Thus, the SMRF constructs may be used as antibacterial compounds, and can be used to inhibit bacterial growth and/or infection.

In one or more embodiments, the SMRF constructs can be used in methods for partially or completely silencing endogenous genes in an animal, such as insects, reptiles, amphibians, fish (e.g., zebrafish), poultry, and mammals, such as rodents (e.g., mouse, rat, rabbit), dogs, cats, pigs, horses, cows, goats, sheep, monkeys, primates, and humans. The SMRF constructs can be formulated for administration using pharmaceutically-acceptable vehicles or excipients. Nucleic acid constructs are particularly suited for delivery via various nanoparticles, liposomes, viral vectors, or vesicles as the delivery vehicles for encapsulating the constructs. In general, a plurality of the SMRF constructs are dispersed in a pharmaceutically-acceptable carrier. The term carrier is used herein to refer to diluents, excipients, vehicles, and the like, in which the constructs may be dispersed for administration. Suitable carriers will be pharmaceutically acceptable. As used herein, the term “pharmaceutically acceptable” means not biologically or otherwise undesirable, in that it can be administered to a subject without excessive toxicity, irritation, or allergic response, and does not cause unacceptable biological effects or interact in a deleterious manner with any of the other components of the composition in which it is contained. A pharmaceutically-acceptable carrier would be selected to minimize any degradation of the constructs or other agents and to minimize any adverse side effects in the subject. Pharmaceutically-acceptable ingredients include those acceptable for veterinary use as well as human pharmaceutical use and will depend on the route of administration. For example, compositions suitable for administration via injection are typically solutions in sterile isotonic aqueous buffer. Exemplary carriers include aqueous solutions such as normal (n.) saline (˜0.9% NaCl), phosphate buffered saline (PBS), sterile water/distilled autoclaved water (DAW), various oil-in-water or water-in-oil emulsions, as well as dimethyl sulfoxide (DMSO), other acceptable vehicles, and the like.

The composition can comprise a therapeutically effective amount of the SMRRT silencing constructs dispersed in the carrier. As used herein, a “therapeutically effective” amount refers to the amount that will elicit the biological or medical response of a tissue, system, or subject that is being sought by a researcher or clinician, and in particular elicit some desired therapeutic effect as against the condition such as by reducing the target RNA transcripts. One of skill in the art recognizes that an amount may be considered therapeutically “effective” even if the condition is not totally eradicated or prevented, but it or its symptoms and/or effects are improved or alleviated partially in the subject. In some embodiments, the composition will comprise from about 5% to about 95% by weight of the SMRRT silencing constructs described herein, and preferably from about 30% to about 90% by weight of the SMRRT silencing constructs, based upon the total weight of the composition taken as 100% by weight. Encapsulation techniques can also be used to facilitate delivery of the SMRRT silencing constructs. Other ingredients may be included in the composition, such as adjuvants, other active agents, preservatives, buffering agents, salts, other pharmaceutically-acceptable ingredients.

In use, a therapeutically-effective amount of the SMRRT silencing constructs is administered to a subject. In some embodiments, a composition comprising a therapeutically-effective amount of constructs are administered to a subject. The disclosed embodiments are suitable for various routes of administration, depending upon the particular carrier and other ingredients used. For example, the SMRRT silencing constructs can be injected intramuscularly, intraperitoneally, subcutaneously, intradermally, or intravenously. They can also be administered via mucosa such as intranasally or orally. The compounds or compositions can also be administered through the skin via a transdermal patch, or topically applied to dermal and epidermal-based cancers or viral infections. A researcher, clinician, or medical practitioner can determine the appropriate dosage amount and timing regimen depending upon various factors such as the age, weight, and overall health of the subject, as well as the severity and stage of the condition to be treated. The SMRRT silencing constructs can be administered, daily, ever other day, weekly, monthly, and the like.

In some embodiments, the SMRRT silencing constructs (or compositions) can be provided in unit dosage form in a suitable container. The term “unit dosage form” refers to a physically discrete unit suitable as a unitary dosage for human or animal use. Each unit dosage form may contain a predetermined amount of the SMRRT silencing constructs (and/or other active agents) in the carrier calculated to produce a desired effect. A kit comprising the SMRRT silencing constructs is also disclosed herein. The kit further comprises instructions for administering the SMRRT silencing constructs to a subject. The SMRRT silencing constructs can be provided as part of a dosage unit, already dispersed in a pharmaceutically-acceptable carrier, or provided separately from the carrier. The kit can further comprise instructions for preparing the SMRRT silencing constructs for administration to a subject, including for example, instructions for dispersing the SMRRT silencing constructs in a suitable carrier.

It will be appreciated that therapeutic and prophylactic methods described herein are applicable to humans as well as for veterinary use for any suitable animal, including, without limitation, dogs, cats, and other companion animals, as well as, rodents, primates, horses, cattle, pigs, etc. The methods can be also applied for clinical research and/or study.

The method comprises delivering to the target locus a SMRRT silencing construct according to the various embodiments herein, wherein binding between the complementary guide fragments from the SMRRT silencing construct and the target locus induces the modification of the target locus of interest. In particular, binding of the guide crRNA fragment with its complementary target yields double-stranded RNA which triggers the endogenous RNA processing machinery of the host cell or organisms. This leads to degradation of the RNA target and thus reduction in downstream gene products.

The RNA-targeting constructs can also be used for inhibition of RNA viruses. For example, the SMRRT silencing constructs can also be used to inhibit RNA viral replication and infection in plants. It is readily appreciated that the antisense silencing RNAs are capable of hybridizing directly to the genomic RNA ((+) RNA strand) of the target virus by base pairing, and so of inhibiting the genomic RNA either prior to or during replication, whereas the sense silencing RNAs are capable of hybridizing to the (−) replicative strand of the target virus which is produced during replication of the target virus, and thereby are capable of inhibiting replication of RNA viruses during viral replication, or subgenomic RNAs, thereby capable of degrading viral messenger RNA, i.e., post-transcriptional degradation. The constructs can be introduced into plant cells or tissues to inhibit viral replication in the plants.

Additional advantages of the various embodiments of the invention will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments, but is not necessarily included. Thus, the present invention encompasses a variety of combinations and/or integrations of the specific embodiments described herein.

As used herein “regulatory sequences” refers to promoters, enhancers, and other expression control signals that direct constitutive expression of a nucleotide sequence. Such elements may be host-specific or may drive expression broadly across various host cell types. Such elements may also be inducible and direct expression only under certain conditions (e.g., active or “on” only under an external stimulus, tissue-specific, or developmentally determined parameter). Advantageously, regulatory sequences can be selected to achieve targeted expression of the constructs. That is, a native promoter can be identified for the host organism that has a specific expression pattern (e.g., time or tissue specific expression). Using this promoter in the constructs ensures that the construct is thus, only expressed in the desired tissue or at the desired time. That way, you only get target mRNA silencing in that tissue (e.g., only in the roots, or root hairs, or only in the root epidermis in the elongation zone, etc.) or at the particular time of development. In this way, the constructs can be selectively activated.

A “host cell” or “target cell” as used herein, refers to the cell into which the constructs have been introduced, include the progeny of the original modified cell. A “host” or “subject” as used herein refers to an individual organism targeted for altered gene expression via SMRF manipulation of RNA targets. Likewise, a “host” or “target” population refers to a plurality of individual host organisms which may be targeted for altered gene expression through SMRF manipulation of RNA targets.

The term “control” when used with respect to control plants or other organisms includes wild-type (native) plants or organisms, as well as cultivars and genetically altered plants that otherwise contain a wild-type, non-modified, or native (endogenous) gene targeted for silencing (inhibition) according to the invention. Such control plants or organisms are compared to “modified” plants or organisms which have been treated with the SMRF constructs according to the invention.

The “inhibition,” “silencing,” or “knock down” of the expression, activity, or function of a gene, as used herein, is intended to refer to any suitable method of reducing or even completely suppressing protein expression from a gene or a coding sequence, including methods of reducing the levels of protein produced as a result of gene transcription to mRNA and subsequent translation of the mRNA. Gene inhibition may be effective against a native gene associated with a trait, e.g., to provide the organism with a diminished level of a protein encoded by the native gene or with reduced levels of an affected metabolite.

The terms “vector” or “expression cassette” refer to nucleic acid molecules that transfer DNA or RNA segment(s) from one cell to another. The term includes recombinant DNA or RNA molecules containing a desired coding sequence(s) and appropriate nucleic acid sequences (e.g., promoters) necessary for the expression of the operably linked coding sequence in a particular host organism. In one or more embodiments, the vector is a plasmid. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses (AAVs)). Viral vectors also include polynucleotides carried by a virus for transfection into a host cell.

A “sense” strand of nucleic acid construct refers to a strand that is transcribed by a cell in its natural state into a “sense” mRNA. The term “antisense” refers to a DNA sequence whose sequence of deoxyribonucleotide residues is complementary to all or part of the sequence of deoxyribonucleotide residues in a sense strand. Thus, an “antisense” sequence is a sequence having the same sequence as the non-coding strand in a DNA duplex. With respect to RNA, the term “antisense” refers to an RNA transcript that is complementary to all or part of a target primary transcript or mRNA and that blocks the expression of a target gene by interfering with the processing, transport and/or translation of its primary transcript or mRNA. The complementarity of an antisense RNA or DNA may be with any part of the specific gene or transcript, i.e., at the 5′ non-coding sequence, 3′ non-coding sequence, introns, or the coding sequence.

The present description uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

Finally, as used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

### EXAMPLES

The following examples set forth methods in accordance with the invention. It is to be understood, however, that these examples are provided by way of illustration and nothing therein should be taken as a limitation upon the overall scope of the invention.

### Introduction

The development of RNA-targeting CRISPR provides potential advantages over DNA editing, such as avoiding pleiotropic effects of genome editing and expanded function including anti-viral immunity. Here, we report the use of CRISPR-Cas13 in plants to reduce both viral and endogenous RNA. Unexpectedly, we discovered that crRNA designed to guide Cas13 could, in the absence of the Cas13 protein, cause substantial reduction in RNA levels as well. We demonstrate this Cas13-independent silencing in four plant species, including stable transgenic Arabidopsis. We determined this was the result of crRNA dependent guide-induced gene silencing (GIGS) and show that GIGS utilizes endogenous RNAi machinery. From this work, we have further explored guide design and re-termed these constructs as “short multiple repeat fragments” (SMRFs) for use in short multi-repeat RNA targeting (SMRRT), since the designed RNA constructs are no longer “guiding” anything to the target. Our results demonstrate that SMRRT silencing is active across a range of plant species and provides a novel and flexible approach to RNA reduction. These findings, along with similar evidence in mosquitoes, suggests that GIGS is potentially active across many eukaryotes.

### Example 1

An alternative approach to targeting DNA is the manipulation of RNA. RNA plays a central role in cellular dynamics, mediating phenotype and trait development across eukaryotes. Manipulating RNA does not require editing the DNA of multi-copy sequences, and could be developed to overcome negative pleiotropy. As such, we initially sought to develop the class II type VI CRISPR-Cas13 system, where the Cas13 nuclease specifically binds target single-stranded (ss)RNA in a CRISPR RNA (crRNA) guided manner, to function as an introduced anti-viral immune system and more generally as a platform to manipulate endogenous plant mRNA. Cas13a (formerly C2c2), first characterized by Abudayyeh et al. (2016), Cas13d, first characterized by Konermann et. al. (2018).

Programmable CRISPR-Cas13 machinery requires a CRISPR-Cas13 protein, and CRISPR-RNA (crRNA) containing a 37 nucleotide (nt) direct repeat (DR), followed by a 28 nt spacer/guide to direct the Cas13 protein towards a specific mRNA sequence.

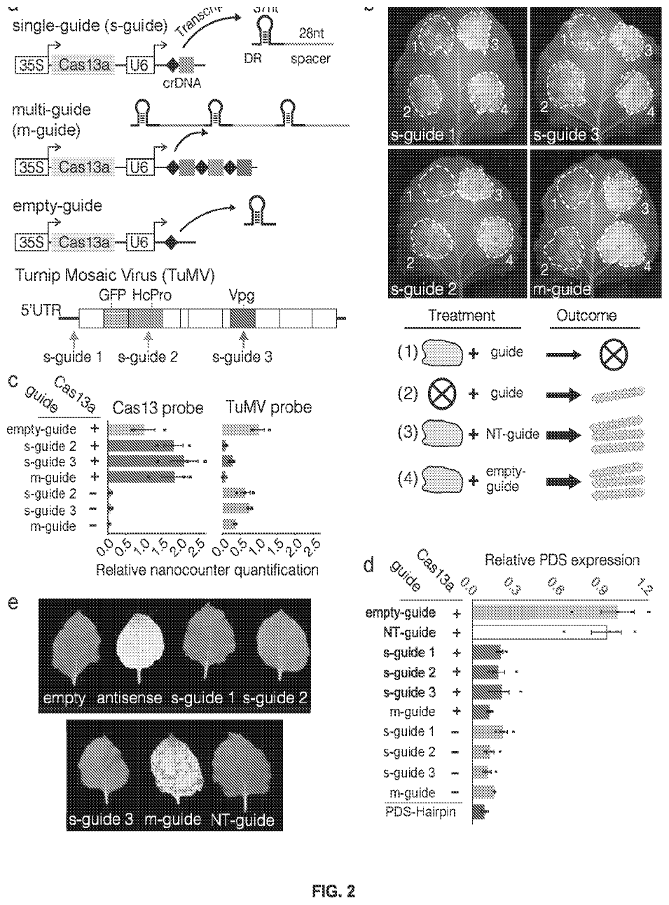

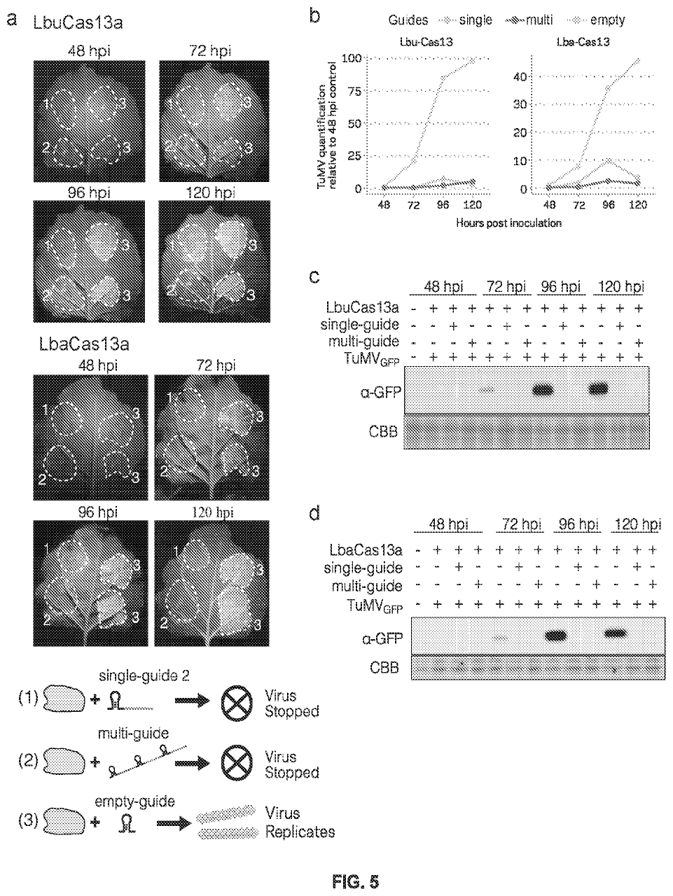

Two Cas13a proteins (from Lachnospiraceae bacterium (LbaCas13a) or Leptotrichia buccalis (LbuCas13a)) were co-expressed with Turnip mosaic virus (TuMV) expressing GFP in Nicotiana benthamiana leaves using Agrobacterium-mediated transient expression. The Cas13 proteins were expressed either with a single-guide crRNA containing antisense sequence to one region of the TuMV genome (single-guide), a multi-guide crRNA containing sequences against three regions of the genome (multi-guide), or an empty-guide, which contained the direct repeat (DR) crRNA sequence alone (FIG. 2a). Expression of either Cas13a protein with the single- or multi-guide crRNA reduced viral accumulation by 72 hours post inoculation (hpi) (FIG. 5a). Virus accumulation was reduced by approximately 90% at 120 hpi, and TuMV interference by Cas13a was dependent on the expression of a crRNA with complementary sequence (FIG. 5b-d).

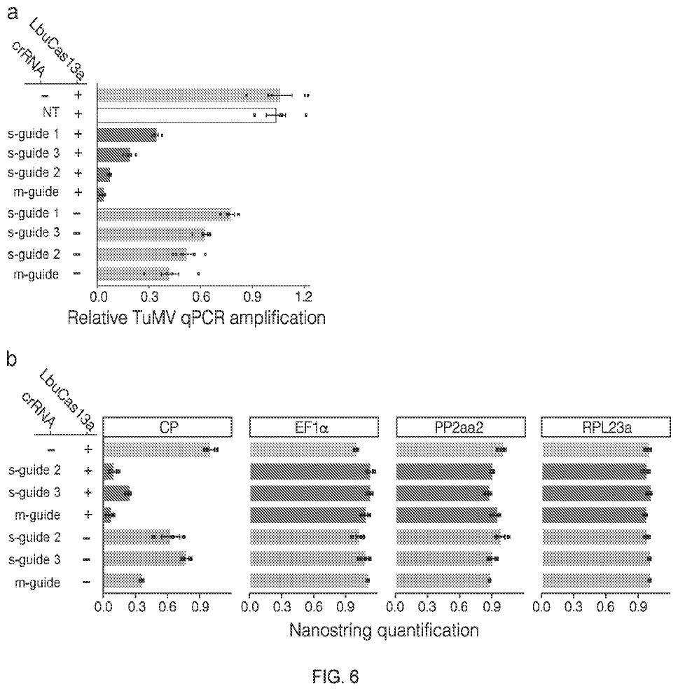

In traditional CRISPR-Cas experiments, the negative controls characterizing cells expressing the sgRNA or crRNA alone, without Cas, are generally omitted due to the assumption of Cas-dependence. Unexpectedly, we observed that expression of a single-guide or multi-guide crRNA alone, in the absence of the Cas13a protein, nonetheless inhibited viral accumulation (FIG. 2b and FIG. 6a). This was evidenced by reduced viral genome and derived protein accumulation. Viral RNA was also directly quantified using two independent NanoString nCounter probes, which allowed direct RNA quantification without the creation of complementary (c)DNA. Probes against two different regions of the TuMV genome confirmed that the single-guide and multi-guide caused virus interference when expressed with Cas13a, but also when expressed alone, in the absence of Cas13a (FIG. 2c and FIG. 6b). The NanoString quantification indicated that LbuCas13a plus guides provided greater viral interference compared to the single- or multi-guide alone. Among the samples expressing guide crRNA alone, the multi-guide consistently caused the greatest TuMV reduction compared to the single-guides (FIG. 2b,c and FIG. 6a,b)

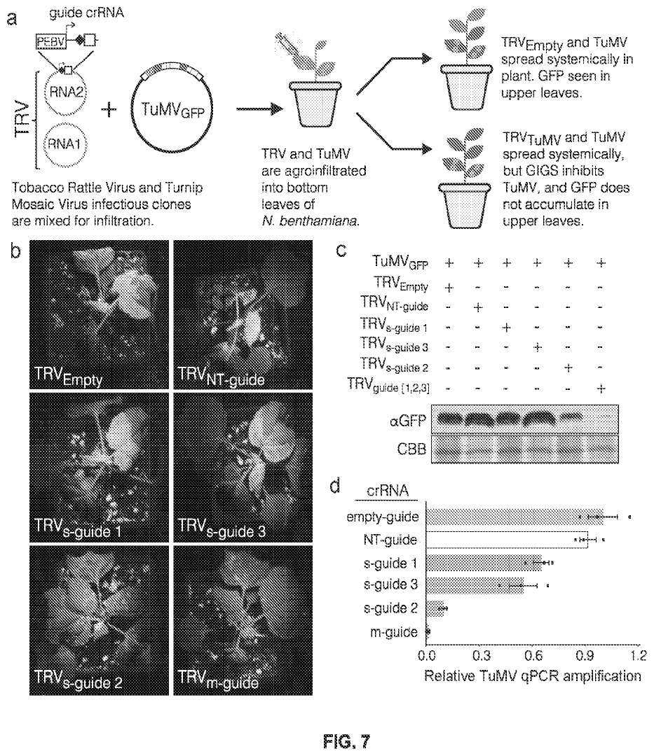

To determine whether the guide crRNA alone can function systemically, GIGS-mediated TuMV interference was tested using the tobacco rattle virus (TRV) expression system. Plants were co-inoculated with TuMV expressing GFP and TRV, which systemically produced single- and multi-guide crRNA in the absence of Cas13 (FIG. 7a). At 7 days post inoculation (dpi), GFP-fluorescent from TuMV was observed in the upper systemic leaves of plants co-inoculated with either TRV expressing an empty-guide or a non-targeting (NT)-guide, which showed that systemic TRV delivery alone did not interfere with TuMV replication, movement, or translation (FIG. 7b-d). Samples expressing the two single-guides, guide 1 and 3, also accumulated visible GFP fluorescence in upper, non-inoculated leaves, indicating the spread of TuMV. Unexpectedly, TRV expressing either single-guide 2 or the multi-guide caused a significant reduction in GFP-fluorescence in the upper systemic leaves (FIG. 7b,c). Quantitative assessment of TuMV accumulation by qPCR showed an approximately 90% reduction in TuMV accumulation in samples expressing single-guide 2 and the multi-guide (i.e., GIGS) (FIG. 7d). Moreover, qPCR revealed an approximate 30% to 40% reduction in TuMV levels when TRV expressed single-guide 1 or -guide 3, which was not obvious from visual inspection of GFP fluorescence. This may reflect complicated translation mechanisms viruses employ, such as internal ribosome entry, in which the viral molecule was targeted by GIGS and partially interfered with, while intact GFP open reading frame sequence was still translated. These results indicate that GIGS can cause systemic TuMV interference, but that not all crRNA target sequences work as effectively. Variation for crRNA effectiveness has been reported for Cas13-dependent RNA targeting, likely caused by secondary structure and accessibility of the target RNA.

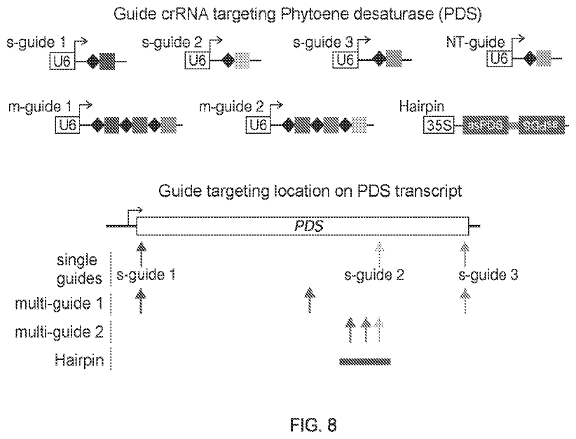

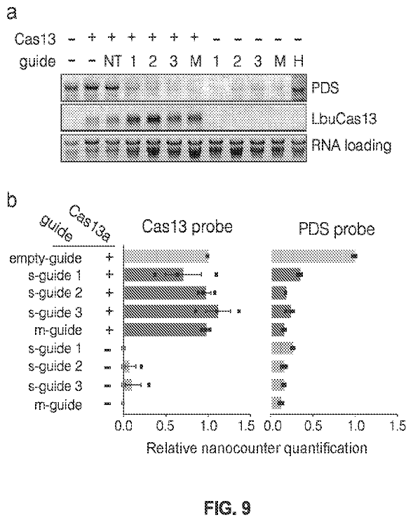

Viruses manipulate host physiology and have unique features unlike host derived RNAs, making it possible that the observation of guide crRNA alone silencing was a phenomena strictly related to viral RNA. To test this hypothesis, we targeted endogenous phytoene desaturase (PDS) mRNA with single-guide and multi-guide crRNA with and without LbuCas13a (FIG. 8). Agrobacterium-mediated expression of single- and multi-guide crRNA with and without LbuCas13 caused a significant reduction in PDS transcript levels compared to expressing LbuCas13a alone or with a NT-guide (FIG. 2d). The resulting mRNA reduction (75-85%) was consistent across the tested samples, comparable to a PDS-hairpin construct known to induce RNAi (FIG. 2d). The reduction in PDS mRNA was confirmed by northern blot, which showed a clear reduction for PDS signal for both LbuCas13a-depenedent and GIGS compared to expressing LbuCas13a alone, with a NT-guide, or from an untreated leaf (FIG. 9a). Direct RNA quantification by NanoString further confirmed a significant reduction for the PDS transcript for samples expressing the PDS targeting guides with or without the expression of Cas13a (FIG. 9b). These results show the phenomena is not specific for viral RNA and extend the observation of guide crRNA alone silencing to endogenous genes.

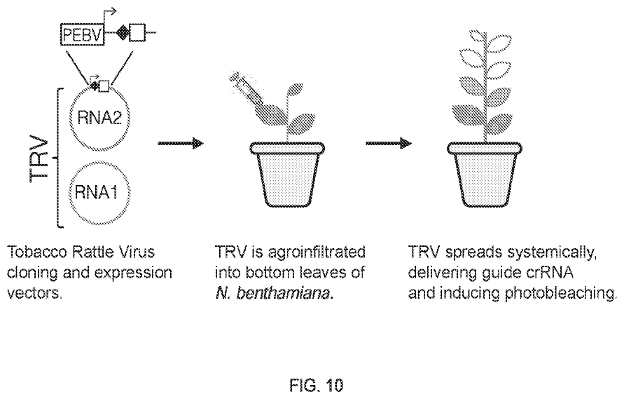

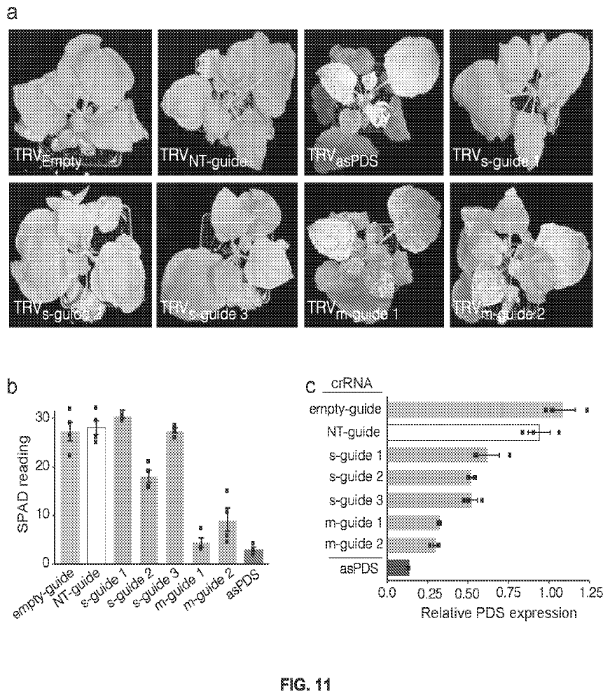

To test if guide crRNA alone silencing acts systemically on endogenous genes, TRV expressing guides targeting endogenous PDS mRNA were infiltrated into N. benthamiana (FIG. 10). Under the hypothesis that GIGS can act systemically on endogenous genes, the hope was that TRV-delivered guides would result in photobleaching in TRV-infected tissues. Three single-guide crRNA, targeting different regions of PDS, did not exhibit significant photobleaching (FIG. 2e). However, two multi-guide constructs targeting different PDS regions displayed substantial photobleaching in systemic leaf tissue (FIG. 2e and FIG. 11a). Interestingly, the visible photobleaching pattern induced by the anti-sense hairpin fragment (i.e. RNAi) and that induced by GIGS were not the same (FIG. 2e and FIG. 11a). While the antisense RNAi photobleaching was strong in the upper, youngest leaves, GIGS induced photobleaching was not visible in the upper most leaves, and the photobleaching occurred in more distinct segments causing a patchy appearance. Quantifying the photobleaching to confirm the phenomena, SPAD meter readings showed a significant reduction in chlorophyll content for samples expressing the multi-guide crRNAs and the antisense PDS fragment (FIG. 11b). Plants that expressed single-guide 2 were yellow and also showed a reduced SPAD reading (FIG. 11a,b). Quantifying PDS transcripts with qPCR showed that the PDS transcript level was reduced (30-45%) for the three single-guides, and to a greater extent by the multi-guides (65-70%) and the antisense construct (85%) (FIG. 11c). It is not clear why single-guide 1 and 3 caused a reduction in PDS mRNA levels, but did not result in visible photobleaching or SPAD meter reductions, but we note that the reduced PDS mRNA levels are consistent with that seen using Agrobacterium-mediated spot infiltration (e.g., FIG. 2d and FIG. 9). Also, the results clearly showed that GIGS induced by multi-guide constructs caused the greatest reduction in target transcript levels for both virus and endogenous RNA targeting.

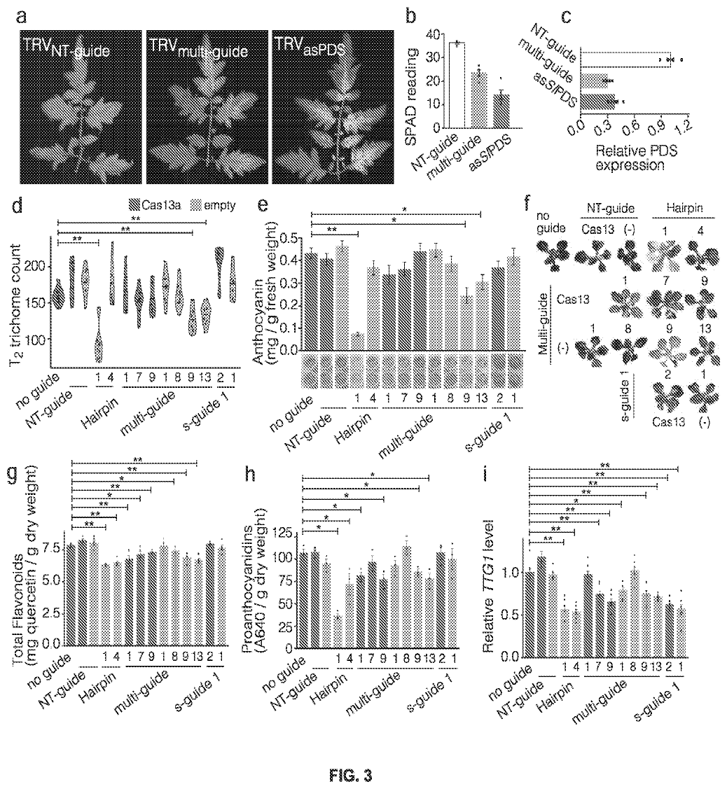

An important question is whether GIGS is specific to N. benthamiana or is more broadly active in plants. To test this, multi-guide crRNA constructs were developed to target PDS in tomato (Solanum lycopersicum), which were delivered using TRV, along with a NT-guide and an antisense PDS control. We observed visible photobleaching in upper leaves of S. lycopersicum plants following systemic movement of TRV expressing a multi-guide targeting S. lycopersicum PDS, although the photobleaching was not as widespread as that produced by the antisense PDS construct (FIG. 3a). Quantifying chlorophyll levels and the PDS transcript indicated that photobleached tissue from GIGS and antisense expressing TRV both had substantially lower levels compared to the control (FIG. 3b,c). These results show that GIGS is active outside of N. benthamiana, possibly extending to other plants in the Solanaceae family.

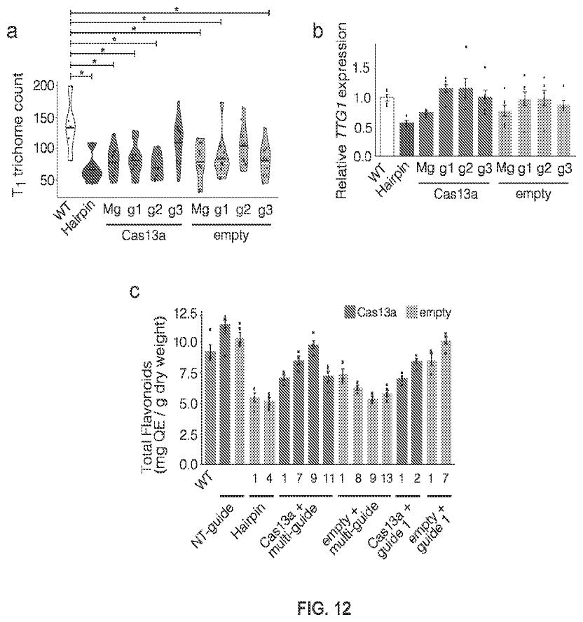

Another important question is whether GIGS requires bacterial or viral machinery (i.e., proteins) introduced during transient expression or if GIGS functions in stable transgenics through plant endogenous machinery. To test this, we transformed Arabidopsis thaliana (Col-0) with single-guide and multi-guide crRNA targeting the pleiotropic regulator TRANSPARENT TESTA GLABRA1 (TTG1), both with and without LbuCas13a. The TTG1 gene encodes a WD40 repeat protein, which interacts with MYB and bHLH transcription factors required for normal trichome and root hair development, along with seed proanthocyanidin and vegetative anthocyanin production. The average trichome counts for multiple independent T₁ plants that expressed LbuCas13a with either single-guide or multi-guide crRNA had significantly fewer trichomes compared to wild-type, and importantly, plants expressing single-guides and the multi-guide crRNA, without Cas13, also had significantly fewer trichomes on average (FIG. 12a). The TTG1 transcript was quantified in T₁ plants and was highly variable across the transformed lines (FIG. 12b). Individual plants were selected, self-fertilized and seeds from T₁ plants showed reduced total flavonoids in both Cas13 and GIGS lines, consistent with reduced TTG1 (FIG. 12c).

We assessed whether GIGS would function in progeny inheriting guides by characterizing individual lines in the T₂ and T₃ generations for altered TTG1 phenotypes. Trichome counts of the seventh leaf (from ten plants per line) indicated that two GIGS lines (i.e., expressing only a multi-guide crRNA targeting TTG1), and one of the hairpin expressing lines had significantly fewer trichomes compared to the transformation control expressing Cas13a alone (FIG. 3d). Individual transformed lines were subjected to sucrose and light stress to induce leaf anthocyanin production, and we again observed that two lines expressing multi-guide crRNA targeting TTG1 (i.e., GIGS) displayed significantly reduced leaf anthocyanin levels, along with a hairpin expressing line (FIG. 3e,f). Quantification of total seed flavonoids showed a significant but modest reduction compared to the control line, for both Cas13 expressing and GIGS lines along with both hairpin expressing lines (FIG. 3g). As total flavonoid quantification measures products of both TTG1-dependent and -independent pathways, we measured seed proanthocyanidins to more accurately measure the impact of TTG1 reduction. This analysis identified the same transformed lines as having significantly lower proanthocyanidins as overall reduced flavonoids, but the level of reduction was more substantial (FIG. 3h).

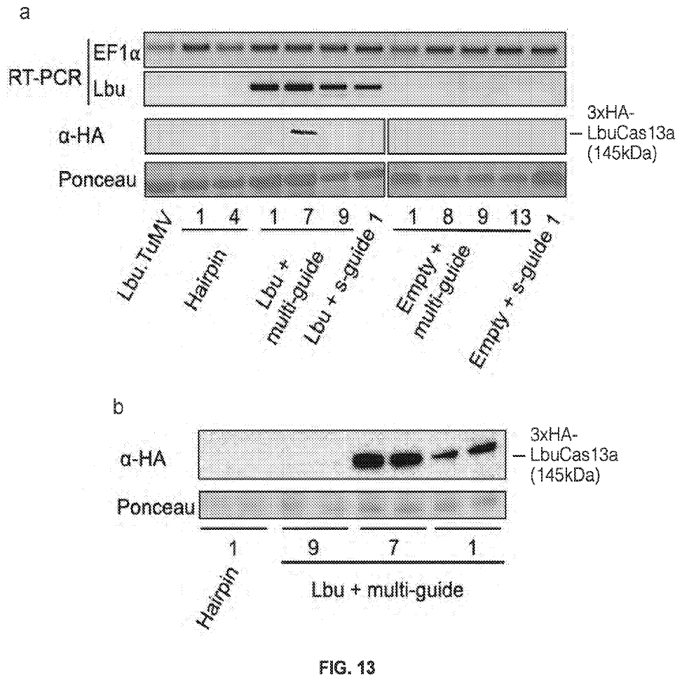

These results indicate heritable phenotypes for multiple traits mediated by both Cas13 and GIGS in stable transgenic Arabidopsis when targeting the pleiotropic regulator TTG1. We do note there was substantial phenotypic variation among lines with the same construct, despite significant reduction in TTG1 levels (FIG. 3i). This is in part explained by variation in transgene expression and translation (FIG. 13). In addition, more complicated mechanisms such as asynchronous TTG1 expression and Cas13 or GIGS expression at the individual cell level, or the effect of incomplete TTG1 silencing on trait manifestation (i.e., kinetics of silencing to produce a phenotype). Optimizing Cas13 and GIGS approaches will be an important step to deliver robust biotechnology platforms for plant research and crop improvement, particularly for tissue- or temporal-specific expression that is difficult to manipulate precisely with CRISPR-Cas9.

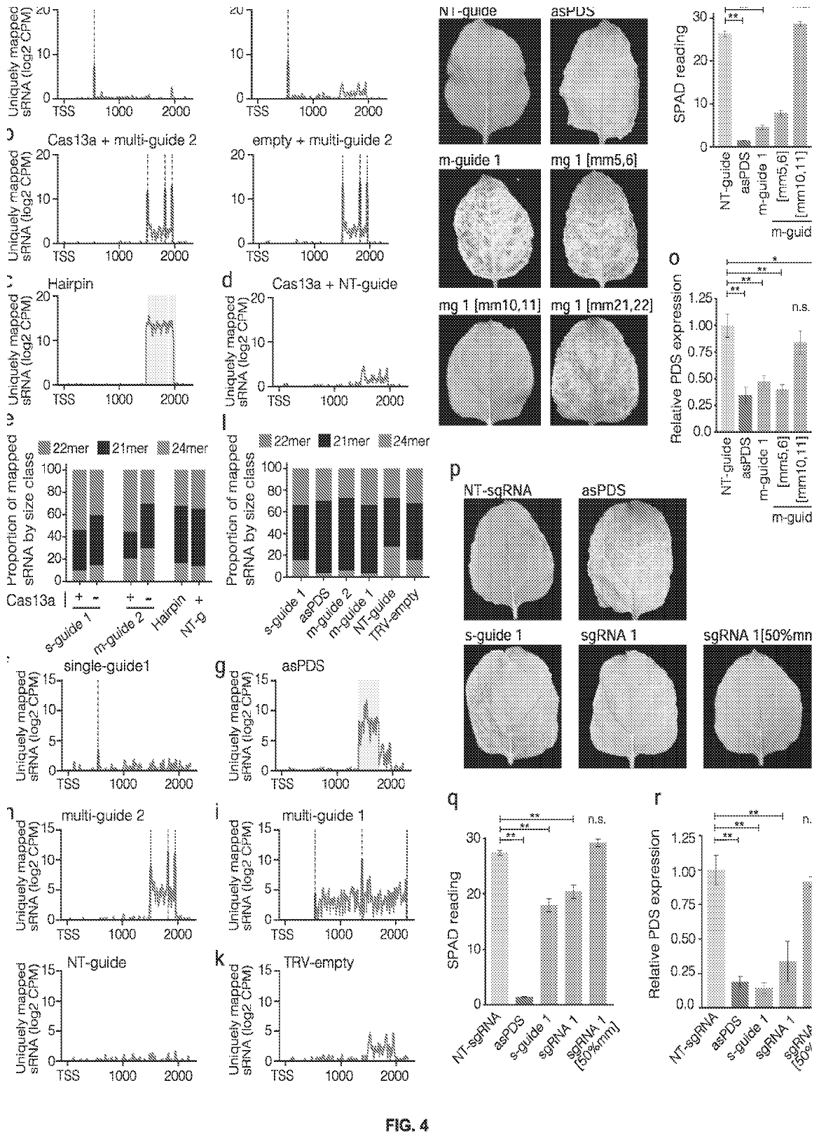

We sought to understand the mechanism giving rise to guide crRNA alone reducing viral and endogenous RNA levels. Given that crRNA are composed of short antisense sequences, it is possible that GIGS functions through components of the endogenous RNA interference (RNAi) pathway. However, the structure of crRNA used here are very different from the hairpin RNA, small interfering RNA (siRNA), or micro RNA (miRNA) used for traditional RNAi, therefore it is not obvious how crRNA might enter or induce RNAi. Alternatively, it is possible that GIGS elicits other endogenous endo- or exonucleolytic RNA degradation pathways. Since small RNA (sRNA) usually in the range of 21- to 24-nucleotides (nt) are a hallmark for RNAi, we reasoned that if GIGS functions through RNAi, abundant sRNA could be identified. To assess this, we conducted small (s)RNA-seq from N. benthamiana samples expressing single and multi-guide crRNA against the endogenous PDS transcript. Mapped sRNA for the single-guide samples showed a single sharp peak at the PDS transcript, which corresponds to the location of the crRNA guide sequence, regardless of Cas13 expression (FIG. 4a). Likewise, the samples expressing the multi-guide crRNA had three distinct peaks of mapped sRNA, each corresponding to the location of the targeting guide sequence. Expectedly, in these samples we also identified many sRNA mapping to the PDS transcript that were independent from the multi-guide target sequence (FIG. 4b). Interestingly, these sRNA were identified only between the 5′ and 3′ boundaries of crRNA targeting sites and do not appear to extend past this region (FIG. 4b). This was similar to the sRNA mapping from the samples expressing the PDS hairpin, which produced ample sRNA between the two ends of the hairpin fragment (FIG. 4c). While the most abundant peaks for the multi-guide crRNA samples corresponded to the guide targets themselves, the identification of thousands of sRNA reads between these target regions suggest the production of secondary sRNA using the GIGS constructs. We do note the presence of background sRNA in the samples where Cas13 was expressed with a NT-guide, which may indicate background read mapping or potentially RNA contamination during library preparation, but regardless, the signal was low (FIG. 4d). Supporting the idea that GIGS results in the production of secondary sRNA through RNAi, we identified more 21 nt sRNA (i.e., siRNA) mapped to the PDS transcript during GIGS (i.e. without the Cas13 protein) than when Cas13 was expressed with the guide (FIG. 4e). To further determine sRNA production during GIGS, a second sRNA-seq experiment was conducted by expressing either a single-guide or one of two multi-guide crRNA in the absence of Cas13 using the TRV vector in N. benthamiana. The mapped sRNA from the single-guide had a clear but small peak corresponding to the guide target sequence, along with other background mapped sRNA (FIG. 4f). In contrast, mapped sRNA from the sample expressing a PDS antisense fragment produced many sRNA, which mapped between the ends of the antisense fragment (FIG. 4g). Both multi-guide crRNAs showed three sharp peaks of mapped sRNA, with each peak corresponding to a guide targeting region (FIG. 4h,i). Importantly, these samples clearly have many mapped sRNA that are independent of the expressed multi-guide sequences, which are not present in the controls, and intervene the regions targeted by the multi-guide crRNA (FIG. 4h-i). We interpret these sRNA to represent secondary sRNA generated in response to multi-guide GIGS. Consistent with these secondary sRNA being generated via components of the RNAi pathway, the length of sRNA mapped to the PDS transcript are predominantly 21 nt for the two multi-guide and antisense fragment samples (FIG. 4l). These results suggest that siRNA and RNAi could be involved in mediating GIGS.

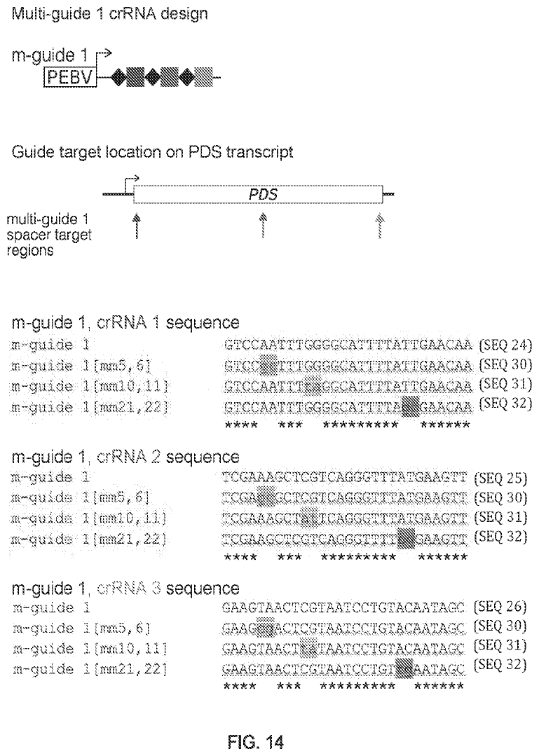

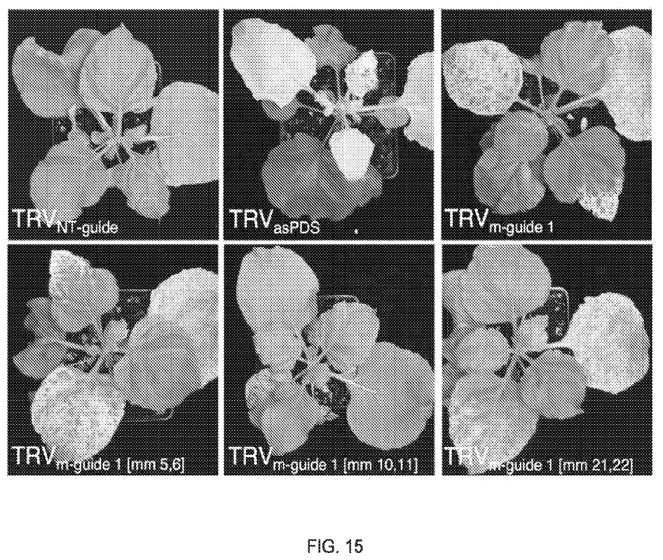

Under the hypothesis that GIGS requires endogenous RNAi machinery, target mRNA reduction would be dependent on ARGONAUTE (AGO) RNA-binding protein(s). AGO proteins are required to form the RNA Induced Silencing Complex (RISC), which carries out the biochemical slicing or translational inhibition of target mRNA. To achieve AGO mediated endonuclease activity, perfect complementary base pairing is required at positions 10 and 11 between AGO-bound siRNA and the target mRNA (i.e., central duplex region). Therefore, if GIGS is dependent on AGO, we predicted that multi-guide crRNA designed to have mismatches at base-pairs 10 and 11 would be blocked for GIGS (i.e., no target mRNA reduction). To test this, multi-guide crRNA that contained specific two base pair mismatches to the PDS mRNA were delivered to N. benthamiana using TRV (FIG. 14). The results show that multi-guide crRNA against PDS with mismatches at the critical region for AGO endonuclease activity (i.e., base pairs 10,11) do not cause photobleaching, while negative control mismatches (i.e., positions 5,6 or 21,22) still elicit photobleaching (FIG. 4m, FIG. 15). Indeed, SPAD meter readings and PDS transcript quantification showed that the multi-guide with mismatches 10,11 were not reduced for the two traits when compared to the controls expressing TRV alone or a NT-guide crRNA (FIG. 4n,o). We note that the mismatches at 5,6 and 21,22 did affect silencing, as the perfectly complementary multi-guide crRNA gave the strongest photobleaching. These mismatches may interfere with other RISC functions, such as target recognition and target mRNA turnover. However, it is clear that mismatches at 10,11 abolish GIGS, while the other mismatches diminish it, suggesting that GIGS functions through one or more endogenous AGO proteins. Additionally, these results suggest that GIGS is mediated by RNA endonuclease reduction and not translational inhibition of target mRNA.

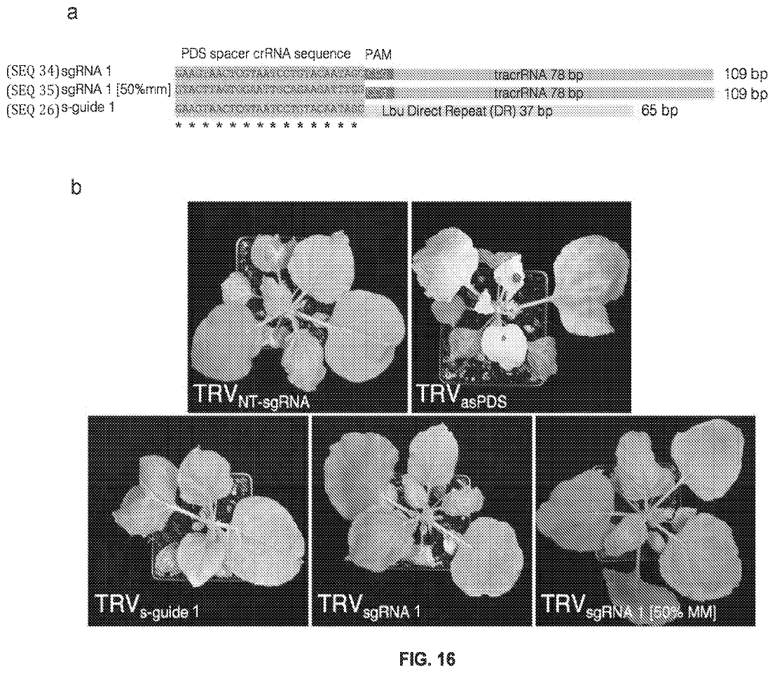

During preparation of this work, it was reported in a mosquito system that crRNA guides from the Cas13b system cause target mRNA reduction in the absence of Cas13b, termed Cas13b-independent silencing. That report does not provide functional data that elucidate the mechanism, but the authors postulate that Cas13b-independent silencing is related to RNAi. We posit that the findings described in mosquito suggest that GIGS functions broadly across eukaryotes. If this is true, we were interested to address if GIGS is active for other guide crRNA, such as for the CRISPR-Cas9 system. To this end, we tested if a single-guide that elicited PDS mRNA reduction when designed as a Cas13 crRNA could additionally reduce target mRNA when designed as a 28 nt Cas9 sgRNA (FIG. 16a). When the Cas9 designed sgRNA was delivered by TRV, we observed subtle yellowing in the leaves compared to TRV expressing a NT-guide, similar to that produced by the Cas13 crRNA design (FIG. 4p). Importantly, a control Cas9 designed sgRNA containing 50% mismatches to the PDS sequence shows no yellowing, indicating that this subtle phenotype is specific (FIG. 4p and FIG. 12b). Quantifying these results, we saw that the sgRNA designed against PDS resulted in approximately 28% reduction in SPAD meter readings, and variable PDS transcript reduction (25-90%) compared to the NT-guide and the 50% mismatch sgRNA controls (FIG. 4q,r).

In summary, we report that Cas13a can effectively reduce targeted viral and endogenous RNA levels. Unexpectedly, we also discovered that the guide crRNA designed for the Cas13a system can also reduce viral and endogenous RNA in the absence of the Cas13 protein (i.e., guide crRNA alone) in the three plant species tested. We also provide evidence that GIGS elicits small RNA and is dependent on AGO for target mRNA reduction, strongly implicating that GIGS functions via endogenous RNAi machinery. Further research is needed to understand how crRNA guides enter or elicit the RNAi pathway, the interplay between RNAi and Cas13-mediated silencing, and how broadly GIGS functions across eukaryotes. The work presented here suggests that GIGS can achieve target RNA silencing using a guide sequence that is shorter than conventional hairpin and anti-sense constructs used in plants, which could be particularly important for targeting multigene families. Additionally, this could afford a higher target specificity compared to RNAi, while avoiding the need to express a Cas13 transgene, which could facilitate crop biotechnology.

### Materials and Methods

### Designing CRISPR-Cas13a Machinery for in Planta Expression

To develop prokaryotic CRISPR-Cas13a machinery as a platform for in planta transcript-silencing, sequences of LbuCas13a and LbaCas13a effectors were N. benthamiana codon optimized along with 3× FLAG tag at the N-terminal were custom synthesized from Genscript (Piscataway, NJ).

TABLE
Plasmids and gene sequences

| Name of sequence | SEQ ID NO: |
|---|---|
| 3x-HA-LbuCas13a | 1 |
| 3X-FLAG-tagged LbaCas13a | 2 |
| Nicotiana benthamiana PDS cDNA | 3 |
| Solanum lycopersicum PDS cDNA | 4 |
| Arabidopsis TTG1 cDNA | 5 |
| PDS-hairpin | 6 |
| pGWB413 | 7 |
| pTRV1 | 8 |
| pTRV2 | 9 |
| TuMV-GFP | 10 |
| pENTR-AtU6:LbuDR:BsaI | 11 |
| pENTR-AtU6:LbaDR:BsaI | 12 |
| pCR8:PEBV:LbuDR:BsaI | 13 |

These fragments were assembled using HiFi DNA assembly (New England Biolabs, Ipswich, MA). The integrity of the constructs was confirmed by Sanger sequencing (Genewiz, South Plainfield, NJ).

Turnip mosaic virus engineered to express GFP (TuMV-GFP) and the endogenous phytoene desaturase (PDS) gene were selected as targets for CRISPR-Cas13a interference. For crRNA designs, Lba- or LbuCas13a specific direct repeats with 28 nucleotide spacer sequences complementary to the target were expressed by the Arabidopsis thaliana U6 promoter.

TABLE
Backbones for cRNA cloning and expression

| Name of | | | SEQ |
|---|---|---|---|
| sequence | Sequence (5′-3′) | Purpose | ID NO |
| AtU6 | TGATCAAAAGTCCCACATCGAT | Express | 14 |
| promoter | CAGGTGATATATAGCAGCTTAG | crRNA | |
| | TTTATATAATGATAGAGTCGAC | | |
| | ATAGCGATT | | |
| PEBV | AATTCGAGCATCTTGTTCTGGG | Express | 15 |
| promoter | GTTTCACACTATCTTTAGAGAA | crRNA | |
| | AGTGTTAAGTTAATTAAGTTAT | using | |
| | CTTAATTAAGAGCATAATTATA | pTRV2 | |
| | CTGATTTGTCTCTCGTTGATAG | | |
| | AGTCTATCATTCTGTTACTAAA | | |
| | AATTTGACAACTCGGTTTGCTG | | |
| | ACCTACTGGTTACTGTATCACT | | |
| | TACCCGAGTTAACGAG | | |
| Lbu crRNA | GATTTAGACCACCCCAAAAATG | Cloning | 16 |
| cloning | AAGGGGACTAAAACAaGAGACC | crRNA | |
| backbone | TTTTTTTTTTGAGACC | | |
| Lba crRNA | GCTGGAGAAGATAGCCCAAGAA | Cloning | 17 |
| cloning | AGAGGGCAATAACGGTCTCGTA | crRNA | |
| backbone | ACTTTTTGAGACC | | |

For TuMV targeting, three single crRNAs targeting different regions of TuMV namely 5′ untranslated region (5′ UTR), Helper component Proteinase (HcPro), viral genome linked protein (Vpg), and a poly crRNA containing aforementioned individual crRNAs in an array were designed and constructed (FIG. 2).

TABLE
crRNA for targeting of TuMV

| Name | | | SEQ |
|---|---|---|---|
| of | | | ID |
| crRNA | Sequence of crRNA | Purpose | NO: |
| UTR- | GTTTCTGCCTTTGCCTCTTAC | Targeting | 18 |
| crRNA | CTTTCGC | TuMV | |
| HcPro- | CTGGGAAATCTTGTTGCGAAA | Targeting | 19 |
| crRNA | GGACTTC | TuMV | |
| Vpg- | TTGTGTTTGCTTTAATCGTTT | Targeting | 20 |
| crRNA | TGTGTAT | TuMV | |
| TuMV | TAACGTTTCTGCCTTTGCCTC | Targeting | 21 |
| Array | TTACCTTTCGCGCTGGAGAAG | TuMV | |
| (Lba) | ATAGCCCAAGAAAGAGGGCAA | | |
| | TAACCTGGGAAATCTTGTTGC | | |
| | GAAAGGACTTCGCTGGAGAAG | | |
| | ATAGCCCAAGAAAGAGGGCAA | | |
| | TAACTTGTGTTTGCTTTAATC | | |
| | GTTTTGTGTATTTTTTT | | |
| TuMV | AACAGTTTCTGCCTTTGCCTC | Targeting | 22 |
| Array | TTACCTTTCGCGATTTAGACC | TuMV | |
| (Lbu) | ACCCCAAAAATGAAGGGGACT | | |
| | AAAACACTGGGAAATCTTGTT | | |
| | GCGAAAGGACTTCGATTTAGA | | |
| | CCACCCCAAAAATGAAGGGGA | | |
| | CTAAAACATTGTGTTTGCTTT | | |
| | AATCGTTTTGTGTATTTTTTT | | |
| NS- | CCTTTCGGTACTTCGTCCACA | Non- | 23 |
| crRNA | AACACAA | targeting | |

Similar to TuMV, the PDS transcript was targeted using three single crRNAs namely, s-guide 1, s-guide 2, and s-guide 3 and a multi-guide crRNA containing the three single guides.

TABLE
crRNA sequences for
targeting of Nicotiana benthamiana PDS

| | | | SEQ |
|---|---|---|---|
| Name of | | | ID |
| crRNA | Sequence (5′-3′) | Purpose | NO: |
| PDS- | GTCCAATTTGGGGCATTTTATTGAACAA | Targeting PDS | 24 |
| crRNA1 | | | |
| PDS- | TCGAAAGCTCGTCAGGGTTTATGAAGTT | Targeting PDS | 25 |
| crRNA2 | | | |
| PDS- | GAAGTAACTCGTAATCCTGTACAATAGC | Targeting PDS | 26 |
| crRNA3 | | | |
| NbPDS- | AACAGTCCAATTTGGGGCATTTTATTGAA | Targeting PDS | 27 |
| Array1 | CAAGATTTAGACCACCCCAAAAATGAAGG | | |
| | GGACTAAAACATCGAAAGCTCGTCAGGGT | | |
| | TTATGAAGTTGATTTAGACCACCCCAAAAA | | |
| | TGAAGGGGACTAAAACAGAAGTAACTCGT | | |
| | AATCCTGTACAATAGCTTTTTTT | | |
| NbPDS- | AACACCTTTTGACTCAATATGTTCCACAAT | Targeting PDS | 28 |
| Array2 | CGGATTTAGACCACCCCAAAAATGAAGGG | | |
| | GACTAAAACAACAGACATGTCAGCGTACA | | |
| | CACTGAGCAGATTTAGACCACCCCAAAAA | | |
| | TGAAGGGGACTAAAACAGGGAAAAGCTT | | |
| | CGCTAGTTCCTTCATTGTTTTTTT | | |
| NS-crRNA | CCTTTCGGTACTTCGTCCACAAACACAA | Non-targeting | 29 |
| Multi- | AACAGTCCgcTTTGGGGCATTTTATTGA | PDS-poly- | 30 |
| g1[mm5,6] | ACAAGATTTAGACCACCCCAAAAATGA | crRNA with | |
| | AGGGGACTAAAACATCGAgcGCTCGTC | mismatches at | |
| | AGGGTTTATGAAGTTGATTTAGACCAC | 5 and 6 | |
| | CCCAAAAATGAAGGGGACTAAAACAG | nucleotides | |
| | AAGcgACTCGTAATCCTGTACAATAGCtttt | | |
| Multi- | AACAGTCCAATTTtaGGCATTTTATTGAAC | PDS-poly- | 31 |
| g1[mm10,11] | AAGATTTAGACCACCCCAAAAATGAAGGG | crRNA with | |
| | GACTAAAACATCGAAAGCTatTCAGGGTTT | mismatches at | |
| | ATGAAGTTGATTTAGACCACCCCAAAAAT | 10 and 11 | |
| | GAAGGGGACTAAAACAGAAGTAACTtaTAA | nucleotides | |
| | TCCTGTACAATAGCtttt | | |
| multi- | AACAGTCCAATTTGGGGCATTTTAgcGAACA | PDS-poly- | 32 |
| g1[mm21,22] | AGATTTAGACCACCCCAAAAATGAAGGGGA | crRNA with | |
| | CTAAAACATCGAAGCTCGTCAGGGTTTTcgG | mismatches at | |
| | AAGTTGATTTAGACCACCCCAAAAATGAAGG | 21 and 22 | |
| | GGACTAAAACAGAAGTAACTCGTAATCCTGT | nucleotides | |
| | tgAATAGCtttt | | |
| NT-sgRNA | AACACCTTTCGGTACTTCGTCCACAAACACAA | Non-targeting | 33 |
| | CGggaaccattcaaaacagcatagcaagtta | | |
| | aaataaggctagtccgttatcaacttgaaaa | | |
| | agtggcaccgagtcggtgctttt | | |
| sgRNA1 | AACAGAAGTAACTCGTAATCCTGTACAATAG | | 34 |
| | CCGggaaccattcaaaacagcatagcaagtta | | |
| | aaataaggctagtccgttatcaacttgaaaa | | |
| | agtggcaccgagtcggtgcTTTT | | |
| SgRNA1 | AACAGTACTTAGTGGAATTGCAGAAGATTTG | | 35 |
| [50%mm] | GCGggaaccattcaaaacagcatagcaagtta | | |
| | aaataaggctagtccgttatcaacttgaaa | | |
| | aagtggcaccgagtcggtgcTTTT | | |

To create mismatch guides corresponding to PDS multi-guide crRNA, the nucleotide sequence was altered at positions 5-6 bp, 10-11 bp, and 21-22 bp from the 5′ end of each crRNA. A non-targeting crRNA was designed as a negative control. To create the sgRNA1 construct, we assembled the single-guide 3 target sequence with the transactivating crRNA (tracrRNA). The same strategy was used to construct sgRNA1 [50% mm] in which single-guide 3 crRNA had mismatches at every-other nucleotide. The NT-sgRNA negative control contained the Cas9 tracrRNA sequence and a non-plant target sequence.

TABLE
crRNA sequences for targeting of tomato PDS

| Name of | | | SEQ ID |
|---|---|---|---|
| crRNA | Sequence (5′-3′) | Purpose | NO: |
| SIPDS- | AACATAAGCTGAACTACCTTGGACT | Targeting | 36 |
| Array | CTCAAGTGATTTAGACCACCCCAAA | tomato | |
| | AATGAAGGGGACTAAAACATTGAA | PDS | |
| | AGTTCGTCAGGGTTTATAAAGTTGA | | |
| | TTTAGACACCCCAAAAATGAAGGGG | | |
| | ACTAAAACACAAGTAACTCATAATC | | |
| | CTGTACAATAGCTTTT | | |
| NS- | CCTTTCGGTACTTCGTCCACAAACA | Non- | 37 |
| crRNA | CAA | targeting | |
| | | control | |

### Cloning of CRISPR-Cas13a Machinery

A backbone harboring AtU6 promoter sequence with one Lbu or Lba specific direct repeat sequence and BsaI Golden Gate site was custom synthesized (IDT, Coralville, IA) for expressing crRNAs. This backbone was cloned into entry vector pENTR (Thermo Scientific, Waltham MA) using Topo cloning. Spacer sequences were ordered as oligos and cloned using BsaI Golden Gate site. Gateway assembly (Invitrogen) was used to clone the promoter and crRNA cassette into the destination vector pGWB413 containing or lacking Cas13a effector.

Cloning crRNA for TRV Systemic Delivery

For systemic expression of crRNA using TRV, pea early browning virus (PEBV) promoter sequence with LbuCas13a specific direct repeat and BsaI Golden gate site were custom synthesized (IDT, Coralville, IA) and cloned into Gateway entry vector PCR8. Three single guide and multi-guide crRNA sequences targeting NbPDS, and a multi-guide crRNA targeting SIPDS were ordered as oligos and cloned using Golden gate assembly. The cassette harboring PEBV promoter and TuMV, NbPDS, or SIPDS targeting crRNAs was PCR amplified with primers having EcoRI and MluI restriction sites and cloned into EcoRI and MluI digested pTRV2 vector.

TABLE
Sequences used in study

| | | | SEQ |
|---|---|---|---|
| Oligo | | | ID |
| name | Sequence (5′-3′) | Purpose | NO: |
| PDS3for | GGACTGGATGAGAAAGCA | qRT-PCR of | 38 |
| | AGGTGT | PDS | |
| PDS3rev | ATCGAAAGCTCGTCAGGG | qRT-PCR of | 39 |
| | TTTATG | PDS | |
| EF1a-FP | AGCTTTACCTCCCAAGTC | qRT-PCR of | 40 |
| | ATC | EF1a | |
| EF1a-RP | AGAACGCCTGTCAATCTT | qRT-PCR of | 41 |
| | GG | EF1a | |
| qPCR-CP- | GAAGGAGAAGAAGGAGAG | qRT-PCR of | 42 |
| FP | AGAGA | TuMV-CP | |
| qPCR-CP- | CAGAGGTTCCAGCGTTTA | qRT-PCR of | 43 |
| RP | CTT | TuMV-CP | |
| qPCR-P1- | TGAGGACGAGAAGGTTGT | qRT-PCR of | 44 |
| FP | AATG | TuMV-P1 | |
| qPCR-P1- | ATTGGTTGCTGCGCTCTA | qRT-PCR of | 45 |
| RP | | TuMV-P1 | |
| AtEF1α FP | CCGAACAAGGAACAAGTG | qRT-PCR of | 46 |
| | AAAG | EF1α | |
| AtEF1α | GTCCTCATCTTCAATGCC | qRT-PCR of | 47 |
| RP | AGTA | EF1α | |
| AtTTG1 | CGCCTCAGAGCTGTAAAC | qRT-PCR of | 48 |
| qPCR FP | ATA | TTG1 | |
| AtTTG1 | CTCCGAACCAGCCGAATA | qRT-PCR of | 49 |
| qPCR RP | AA | TTG1 | |
| PDS- | GGTCACAAACCGATATTG | PDS northern | 50 |
| Probe-FP | CTGGAGG | | |
| PDS- | ACAGACATGTCAGCGTAC | PDS northern | 51 |
| Probe-RP | ACACTGAGCA | | |
| Lbu- | GCTGAAAATATTTAGGCA | Lbu northern | 52 |
| Probe-FP | GTTGAACTC | | |
| Lbu- | GGAACCCTTTAAGTTCGT | Lbu northern | 53 |
| Probe-RP | CAGCT | | |
| LbuCas13a | GTTCGGGATTATGCGAAA | Lbu RT-PCR | 54 |
| FP | GTGACC | in Arabidopsis | |
| LbuCas13a | ACGGAAAACCTCCAGTTC | Lbu RT-PCR | 55 |
| RP | CTCGC | in Arabidopsis | |
| MluI- | GACTAGCGAATTCAATTC | Amplification | 56 |
| PCR8-RP | GAGCATCTTGTTCTGG | of PEBV- | |
| | | crRNA | |
| | | Cassette | |
| EcoRI- | CAGTACAACGCGTCTGGG | Amplification | 57 |
| PEBV-FP | TCGAATTCGCC | of PEBV- | |
| | | crRNA | |
| | | Cassette | |

Cloning of Intron Hairpin RNAi (hpRNAi) Cassette

For cloning of PDS hpRNAi construct, a 197 bp sequence of PDS gene was custom synthesized as sense and antisense arm along with PDK intron sequence with 25 bp overhang complementarity to pGWB413 vector. All the fragments were assembled using HiFi DNA assembly (New England Biolabs, Ipswich, MA) expressed by the 35S promoter.

Agro-Infiltration of N. benthamiana and Solanum lycopersicum

N. benthamiana plants were grown and maintained in growth chamber at 23° C. with 16-hour day and 8 hour light cycle and 70% humidity. Four week old plants were used for leaf spot agroinfiltration to test Cas13a interference against TuMV-GFP. Binary constructs harboring Cas13a homologs with or without crRNA (targeting TuMV or PDS transcript), TuMV-GFP infectious clone (a gift from Dr. James Carrington) were individually transformed into chemically competent Agrobacterium tumefaciens strain GV3101. Single colonies for each construct were inoculated into LB medium with antibiotics and grown overnight at 28° C. Next day, the cultures were centrifuged and suspended in agroinfiltration buffer (10 mM MgCl2, 10 mM MES buffer pH 5.7 and 100 μM acetosyringone), and incubated at ambient temperature for 2-3 hours. For TuMV interference assay, Agrobacterium cells harboring Cas13a with crRNA targeting TuMV were infiltrated at an OD600 of 1.0 into adaxial side of four week old N. benthamiana leaves using a 1.0 ml needleless syringe. Two days later, Agrobacterium cells harboring TuMV-GFP were infiltrated into same areas at an OD600 of 0.3. After five days, interference activity of Cas13a against the TuMV-GFP was assayed by visualizing GFP in infiltrated leaves under UV light using a hand-held UV lamp (Fisher Scientific, Waltham, MA) and a Nikon camera.

For PDS silencing, leaves of four-week-old N. benthamiana plants were infiltrated with Agrobacterium cultures harboring LbuCas13a with crRNAs targeting PDS and leaf samples were collected at 5 days post inoculation. For TRV mediated crRNA delivery, assays used three-week-old N. benthamiana plants. A single colony of Agrobacterium harboring crRNAs targeting PDS were inoculated into LB medium with antibiotics and grown overnight at 28° C. Next day, the cultures were centrifuged and resuspended into infiltration buffer at an OD600 of 0.6. The cultures were incubated at ambient temperature for 2-3 hours and infiltrated into N. benthamiana. Infiltration of tomato plants was performed similarly to N. benthamiana except that Agrobacterium cells were resuspended into infiltration buffer at an OD600 Of 2.0. The cultures were incubated at ambient temperature for 2-3 hours and infiltrated into three-week-old tomato plants.

RNA Isolation, cDNA Synthesis, qRT-PCR and Northern Blotting

Total RNA was isolated from Agro-infiltrated leaf samples using Trizol (Ambion) method. For first strand cDNA synthesis, DNase treated 1.0 ug total RNA was reverse transcribed using either random hexamers or oligo(dT20) and SuperScript II reverse transcriptase (Thermo Fisher Scientific) according to the manufacturer's instructions. Quantitative PCR was performed using SYBR Select Master Mix (Applied Biosystem) and gene specific primers for PDS and TuMV. EF1α gene was used as internal house-keeping reference for PDS and TuMV qRT-PCR The experiments were repeated three times with three biological and two technical replicates. Relative expression values were plotted using ggplot2 in R. For detection of PDS transcript, 20 μg of total RNA was separated on a denaturing 1.2% agarose gel and blotted on a Hybond-N+(Roche) membrane. RNA was crosslinked using UV light and hybridized with a DIG labelled probe (PCR DIG probe synthesis kit, Sigma). For detection of LbuCas13a the membrane was stripped and probed with DIG labelled Cas13a specific probe and signals detected on a Licor Odyssey imaging system (LI-COR Bioscience, Lincoln, NE).

### Real Time Quantification of PDS and TuMV Transcripts Using Nanocounting Technology

For direct RNA quantification of PDS and TuMV transcripts using NanoString technology, we collected sequence data for different N. benthamiana genes including PDS, three house-keeping genes for normalization (PP2aa2, EF1α, RPL23a), LbuCas13a, HCPro and coat protein.

TABLE
Probes for Nanocounting

| Probe | | SEQ ID | |
|---|---|---|---|
| name | (5′-3′) | NO: | Purpose |
| Tumv-CP | CGTGTATAGGATTAGATGATCGAGGTTTAGA | 58 | For |
| A | GCCACTCTTTTCTCGTATCCAACAGCCACTTT | | TuMV |
| | TTTTCCAAATTTTGCAAGAGCC | | quanti- |
| B | CGAAAGCCATGACCTCCGATCACTCCGCGTT | 59 | fication |
| | GAACGTGTGTTGGATAGATCCGTCTGCTCCG | | |
| | G | | |
| Tumv-HCpro | CGAAAGGACTTCAGTGAACCCTTCTCAATGT | 60 | For |
| A | TCTCGGTTCTGTTCTTCAGCACCGTGTGGACG | | TuMV |
| | GCAACTCAGAGATAACGCATAT | | quanti- |
| B | CGAAAGCCATGACCTCCGATCACTCTGTCAC | 61 | fication |
| | ACATTAGTGTTGGGTTGATGTGCGCTTTCTGG | | |
| | GAAATCTTGTTG | | |
| LbuCas13a | CGCAGTTACGCACATAGGTGTCCAGCTTGTTT | 62 | For |
| A | AACAGCTTATTTTCGATACATCCTCTTCTTTT | | LbuCas13a |
| | CTTGGTGTTGAGAAGATGCTC | | quanti- |
| B | CGAAAGCCATGACCTCCGATCACTCGAAGTC | 63 | fication |
| | TGAAGTGGCTATCTCCCCGTCTTGCAAATAAT | | |
| | AGTTGTACTTGC | | |
| NbPDS | TTGGGTAAGCCCCAAAGAATATGTGCAACCC | 64 | For PDS |
| A | AGTCTCGTACCAATCTCCACAAAGACGCCTA | | quanti- |
| | TCTTCCAGTTTGATCGGGAAACT | | fication |
| B | CGAAAGCCATGACCTCCGATCACTCCCACTG | 65 | |
| | CAACCGATCGTTAATCCCTAGTTCTCCAAAC | | |
| | AGGTTCTGCATAT | | |
| NbEIF1a | CAGGGACGGTTCCAATGCCACCAATCTTGTA | 66 | Internal |
| A | AACATCCTGAAGTGGAAGACCAATTTGGTTT | | control |
| | TACTCCCCTCGATTATGCGGAGT | | |
| B | CGAAAGCCATGACCTCCGATCACTCACCAGG | 67 | |
| | CTTGAGCACACCAGTCTCCACACGACCAA | | |
| NbRPL23A | TCCCATCAGGCCTAATCAAGGTATTGACTTTC | 68 | Internal |
| A | TTTGTCTGGATGCGAACCTAACTCCTCGCTAC | | control |
| | ATTCCTATTGTTTTC | | |
| B | CGAAAGCCATGACCTCCGATCACTCAACGTC | 69 | |
| | CAATGCATCGTAGTCAGGAGTCAACCTCACA | | |
| | TATGCTTTCTTCG | | |
| NbPP2aa2 | CTTGCCAAGTGCAGCACAACCCTCAACAGCT | 70 | Internal |
| A | AATAAACGTACAGAATCCTCAGATAAGGTTG | | control |
| | TTATTGTGGAGGATGTTACTACA | | |
| B | CGAAAGCCATGACCTCCGATCACTCTTGACA | 71 | |
| | ATGACAGGCAGGATGTGTGCAACACAATCCT | | |
| | GTGGCTCCAACAG | | |

The sequence information was utilized to design two probes for each target gene. Total RNA samples (300 ng total RNA) and probe master mix were supplied to the Huntsman Cancer Institute, University of Utah for Nanostring quantification following manufacturer specifications. The nanocounting data was analyzed using the nSolver software.

### Western Blotting

For western blotting, total protein was isolated from Agrobacterium infiltrated leaves using extraction buffer (50 mM Tris-Cl, 1% β-Mercaptoethanol and protease inhibitor cocktail (Roche, Basel, Switzerland)). Total proteins were boiled with loading buffer (100 mM Tris-Cl, 20% Glycerol, 4% SDS, 10% β-Mercaptoethanol and 0.2 mg/ml bromophenol blue) and resolved on 12% SDS-PAGE gel. The proteins were transferred from SDS-PAGE gel to PVDF membrane (GE healthcare, Chicago, IL). Membrane blocking and antibody incubations were performed using iBind western device (Thermo Fisher Scientific, Waltham, MA) according to the instrument manual. Finally, the membrane was treated with ECL Select western blotting detection reagent (GE healthcare, Chicago, IL) and signal was detected with Licor Odyssey imaging system (LI-COR Bioscience, Lincoln, NE).

### Generating Stable Transgenic Arabidopsis Plants

TTG1-targeting three single guides (guide-1, -2, -3) and a multi-guide crRNA, and non-targeting (NT) oligos were annealed and ligated into pENTR backbone containing BsaI Golden gate site.

TABLE
crRNA sequences for targeting TTG1

| | | | SEQ |
|---|---|---|---|
| Name of | | | ID |
| crRNA | Sequence (5′-3′) | Purpose | NO: |
| AtTTG1 s- | TAACGAATCTGGAGCTGAATTATC | Targeting | 72 |
| guide 1 | CATG | TTG1 | |
| AtTTG1 s- | CTACAAGTTCCGAGACGTTTCGGC | Targeting | 73 |
| guide 2 | TCTA | TTG1 | |
| AtTTG1 s- | AGGAGCTGCATTTTGTTAGCAAAA | Targeting | 74 |
| guide 1 | GCAA | TTG1 | |
| TTG1 m- | AACATAACGAATCTGGAGCTGAAT | Targeting | 75 |
| guide | TATCCATGGATTTAGACCACCCCA | TTG1 | |
| | AAAATGAAGGGGACTAAAACACTA | | |
| | CAAGTTCCGAGACGTTTCGGCTCT | | |
| | AGATTTAGACCACCCCAAAAATGA | | |
| | AGGGGACTAAAACAAGGAGCTGCA | | |
| | TTTTGTTAGCAAAAGCAATTTT | | |
| TuMV m- | AACAGTTTCTGCCTTTGCCTCTTA | Non- | 76 |
| guide | CCTTTCGCGATTTAGACCACCCCA | targeting | |
| | AAAATGAAGGGGACTAAAACACTG | control | |
| | GGAAATCTTGTTGCGAAAGGACTT | | |
| | CGATTTAGACCACCCCAAAAATGA | | |
| | AGGGGACTAAAACATTGTGTTTGC | | |
| | TTTAATCGTTTTGTGTATTTTTTT | | |

Gateway assembly was used to transfer guide crRNA to pGWB413 destination vector with or without 3×HA-LbuCas13a. Stable transgenic Arabidopsis plants expressing TTG1 guides with or without LbuCas13a were generated using Agrobacterium-mediated floral dip. Similarly, stable Arabidopsis controls with a NT crRNA, a 197 bp hairpin construct against TTG1 (a gift from Dr. Steven Strauss), and no guide transformation control (only 3×FLAG-LbuCas13a) were generated. One month after floral dip, T₁ seeds were collected and stored at 4° C.

### Arabidopsis Phenotyping

Transformed T₁ Arabidopsis seedlings were identified using rapid selection protocol. Selection was conducted on ½ MS media with a Kanamycin concentration of 100 μg/ml. Positive transformants (n=36) for each TTG1 crRNA with or without LbuCas13a and TTG1 hairpin controls were transferred to soil and grown under optimal conditions. Control Arabidopsis Col-0 plants were germinated on ½ MS media without Kanamycin and transferred to soil. Seventh leaf from ten individual plants for each construct was imaged under a dissecting microscope equipped with a Nikon camera and trichomes were counted using multi-point feature in ImageJ software. For each construct, RNA was extracted from 10^{th} leaf of five individual plants with varying leaf trichomes to quantify TTG1 expression using qRT-PCR. AtEF1α was used as internal house-keeping control for normalizing TTG1 expression. Selected individual plants for each construct were self-pollinated to collect T₂ seed. Five technical replicates of each selected plant/line were used for analyzing total flavonoids, in 5 mg seed, using modified aluminum chloride (AlCl₃) colorimetric method. Flavonoids content was estimated using the following formula: flavonoids (mg/g)=concentration obtained through quercetin calibration curve×(volume of extract/seed weight).

To determine the inheritance of GIGS and Cas13-mediated gene silencing, 10 T₂ plants from selected T₁ lines were transferred to soil after Kanamycin selection. Seventh leaf from 10 individual T₂ plants was imaged for counting leaf trichomes. Statistical comparisons between the transformation control (no guide) and each selected line was performed. TTG1 expression in the top rosette leaf from three individual T₂ plants was analyzed using qRT-PCR. Five individual T₂ plants for each line were self-pollinated to collect T₃ seed. Total flavonoid content was analyzed in T₃ seeds from five independent seed lots (five biological replicates). Similarly, proanthocyanidins content was measured using DMACA-HCl method from three seed lots. Proanthocyanidins were measured at 640 nm and reported as per gram of seed weight. Total flavonoid and proanthocyanidin analyses were repeated twice, the averaged values for each seed lot were used for statistical comparisons. Absorbance of flavonoids and anthocyanin was measured using Thermo Spectronic 3 UV-Visible Spectrophotometer. While absorbance of proanthocyanidins was measured through Synergy H1 Hybrid Multi-Mode Microplate Reader (Agilent Technologies, Winooski, Vermont).

For leaf anthocyanin quantification, one-week-old T₃ seedlings after Kanamycin selection were transferred into ½ MS media+3% sucrose and subjected to light stress (500 μmol m⁻² s⁻¹) for one week. 200 mg of leaf tissue was used for quantifying anthocyanin. Anthocyanin analysis was repeated twice with 5 replicates in each batch. Anthocyanin content was calculated by using following formula (absorbance/35,000×dilution factor×647×1,000 per mg of sample extracted (in mg g−1 fresh weight). Representative plantlets following sucrose treatment showing anthocyanin pigmentation were imaged with a dissecting microscope equipped with a Nikon camera. To test TTG1 expression in T₃ generation, seventh leaf from three individual plants was analyzed using qRT-PCR. To determine the expression of LbuCas13a, RT-PCR was conducted on cDNA synthesized for qRT-PCR. Western blot analysis with HA-tag antibody was conducted on one-week-old T₃ seedlings post Kanamycin selection.

### Example 2

### High-Fidelity RNA Silencing Though Improved Design for Short Multiple Repeat Fragment Silencing

In our previous CRISPR-Cas13 experiments evaluating their ability for TuMV (Tobacco mosaic virus) interference, we observed strong TuMV interference with guide crRNA alone in the absence of LbuCas13a protein. We determined that multi-guide crRNA (short multiple repeat fragments), that targeted three mRNA regions of a single gene, produced stronger silencing compared to a single-guide crRNA. In addition to virus interference, systemic TRV (tobacco rattle virus) expression of guide crRNA against endogenous phytoene desaturase (PDS) gene reduced PDS expression and elicited leaf photobleaching in Nicotiana benthamiana and Solanum lycopersicum. In addition to transient assays, stable transgenic Arabidopsis plants expressing guide crRNA against TTG1 (TRANSPARENT TESTA GLABRA1) RNA resulted in lower TTG1 expression, seedling anthocyanin, grain tannins, and fewer leaf trichomes, demonstrating heritability of the silencing approach. Expressing these SMRRT silencing constructs in a tissue- or age-specific manner offers a potential cis-genic solution to bypass antagonistic pleiotropy in plants.

In the preliminary work, the phenotype reduction elicited by the SMRRT silencing constructs was weaker compared to a conventional TTG1-haripin (RNAi) construct. To utilize SMRRT silencing technology in transcriptome engineering, SMRRT silencing constructs need to be optimized to elicit a phenotype reduction levels similar as RNAi. However, limited knowledge exists on guide crRNA design parameters, such as optimal guide length, number of crRNA in a multi-guide construct, crRNA order, and crRNA copy number for effective GIGS. To improve our understanding of SMRRT guide design parameters, we evaluated different guide design parameters using Tobacco rattle virus (TRV) systemic infection in N. benthamiana. Our results shed like on optimized crRNA length, sequence flexibility for crRNA design, crRNA copy number, and crRNA dosage that could potentially improve SMRRT silencing technology.

This prior work is extended, improving SMRRT silencing by altering crRNA length, number, and structure for use of the constructs in gene silencing in in plants. The improved construct design for the SMRFs, a cis-genic RNA silencing approach, elicited stronger gene silencing with reduced off-targeting effects.

As limited knowledge exists on crRNA guide design parameters to apply these new silencing constructs for crop improvement, we tested different guide design parameters by systemically expressing PDS guide crRNA through agro-mediated Tobacco rattle virus (TRV) infection of Nicotiana benthamiana. Systemic movement of PDS multi-guide for RNA silencing revealed a minimum guide length requirement of 22 nt for SMRRT silencing induction and no additional sequence at the 5′ crRNA region. Interestingly, increasing the number of crRNA fragments in a multi-guide from three to six crRNA fragments against PDS mRNA elicited photobleaching patterns observed with the control antisense PDS construct. In addition to testing a six multi-guide construct against PDS mRNA, increasing guide dosage of single crRNA fragments by expressing three or six copies of the same crRNA sequence elicited an increased PDS silencing with an additional guide dosage.

### Results

Multi-Guide crRNA Fragments do not Need to be Assembled as a Single RNA Molecule for SMRRT Silencing

Our previous research demonstrated that multi-guide crRNA fragments resulted in more viral and endogenous RNA reduction compared to silencing elicited by a single guide. One interpretation is that the more locations an RNA is targeted the greater the RNA degradation. This could be achieved by more DCL-based mRNA-crRNA processing and the generation of more siRNA. An alternate hypothesis however, is that multi-guide crRNA change the targeted RNA's physical conformation upon crRNA-mRNA hybridization that results in target RNA decay. This could be achieved if the crRNA-mRNA duplex was detected as an aberrant RNA structure and triggered non-sense mediated decay (NMD) or possibly de-adenylation or de-capping.

To understand which of these mechanisms explains SMRRT silencing, we modified the structure of our previously tested multi-guide crRNA targeting PDS. Here, the multi-guide crRNA containing three separate crRNA fragments was expressed as a single RNA molecule targeting different mRNA locations (FIG. 17a) and driven by a single promoter. To modify this, we split the crRNA guides into three separate RNA molecules driven by individual promoters (FIG. 17a) in order to test if simultaneous expression of three separately expressed crRNA fragments caused similar target RNA reduction as a multi-fragment crRNA molecule (containing all 3 fragments).

We developed the following nomenclature to refer to the different constructs: m3g3 where 3 defines how many guides (crRNA fragments) are contained in a single RNA molecule, and 3 represents how many different crRNA sequences are present on that RNA molecule. For instance, a multi-guide crRNA with three guides targeting three different sequences is termed m3g3. We expressed either PDS m3g3 or three separate single-guide (s-g) crRNA corresponding to these same PDS sequences in N. benthamiana using agro-mediated TRV infection (FIG. 17b).

Two-weeks after infection, we observed similar visible photobleaching in systemic tissue elicited by PDS m3g3 or the simultaneous expression of the three PDS s-guide crRNA (FIG. 17c). The non-target (NT)-guide control produced no visible photobleaching as expected (FIG. 17c). Supporting the visible photobleaching, chlorophyll content was significantly reduced ˜60% with either PDS m3g3 or three PDS s-guides compared to the NT-guide crRNA (FIG. 17d). Quantifying PDS transcript levels in photobleached tissue using qPCR showed a significant ˜50% reduction in PDS mRNA levels from either PDS m3g3 or three PDS s-guides compared to the NT-guide crRNA (FIG. 17e). These results show that three different crRNA fragments targeting different regions of PDS produced significant transcript reduction, regardless if they were expressed as a single crRNA molecule (i.e. multi-guide) or as three separate single guide crRNAs. These results were unexpected and were contrary to our hypothesis that SMRRT silencing involves target RNA conformational change brought about by multiple binding events of a single multi-guide crRNA construct.

Minimum crRNA Length of 22 nt is Required for Silencing of PDS Target

This new technology relies on short crRNA fragments. We sought to understand the thresholds for fragment length. In N. benthamiana, the shortest nucleic acid homology of 23 nt against green fluorescent protein (GFP) was shown to silence GFP RNA and a minimum length of 33 nt against PDS to elicit a visible photobleaching phenotype. In our previous experiments, we observed a complementary nucleic acid sequence of 28 nt initiated SMRRT silencing of PDS RNA for eliciting a photobleaching response in N. benthamiana and S. lycopersicum. However, the minimum guide crRNA length requirement for induction of SMRRT silencing has not been determined. Based on the hypothesis that SMRRT silencing utilizes plant RNAi pathway for altering targeted gene expression, we predicted that short guide crRNA length (<21 nt) is required for initiation of SMRRT silencing by triggering AGO-mediated dsRNA cleavage.

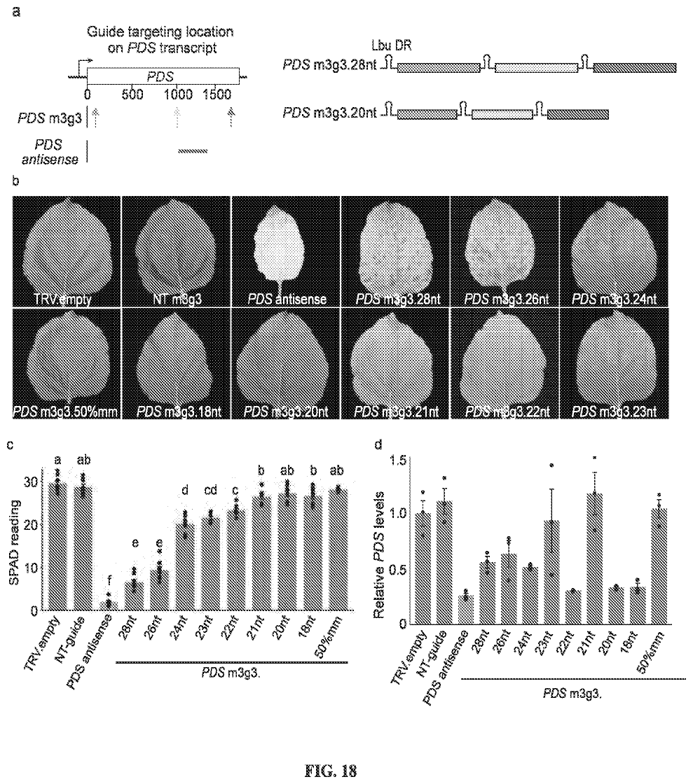

To determine the minimum crRNA length required for induction of GIGS, we evaluated varying lengths, from 18 to 28 nt, for a PDS m3g3 crRNA construct, including an Lbu DR (FIG. 18a). Following systemic TRV movement, SMRRT silencing elicited by PDS m3g3.28nt crRNA showed a photobleaching appearance in lower leaves. The photobleaching phenotype for PDS m3g3.26nt was similar to that of PDS m3g3.28nt (FIG. 18b). Moderate photobleaching was visible around leaf veins in the lower leaves with decreasing visibility for PDS m3g3.24nt, 0.22nt, and 0.23nt crRNA. No visible photobleaching was observed with PDS m3g3.21nt, 20nt, 0.18nt, 50% mismatching (50% mm) crRNA, TRV_{empty}, or NT m3g3 treatments (FIG. 18b). TRV expressing an antisense PDS construct (RNAi) elicited complete photobleaching in the lower leaves. Measuring chlorophyll content via SPAD meter showed 70-80% chlorophyll reduction with PDS m3g3.28nt and 0.26nt, 20-30% reduction with PDS m3g3.24nt, 23nt, and 0.22nt (FIG. 18d). No chlorophyll reduction was seen with PDS m3g3.21nt, 0.20nt, 0.18nt, and 0.50% mm compared to TRV_{empty} control. We observed the largest chlorophyll reduction (93%) with antisense PDS. Collectively, these results indicate that a minimum crRNA length for this construct was 22 nt to see chlorophyll reduction. Moreover, the fragment length was directly correlated to the strength of the reduction observed.

Multi-Guide crRNA do not Require Direct Repeat or Other Specific Sequence Arrangement for Silencing

The traditional CRISPR-Cas13 mediated RNA-targeting system processes precursor crRNA (pre-crRNA) transcripts into mature crRNAs by cleaving before the DR stem region. As SMRRT silencing is independent of a Cas13 protein, there is no specific constraint that “guide sequences” for SMRRT silencing possess the DR sequence, other than the possibility that it could be required to elicit SMRRT silencing. One hypothesis is that the DR stem-loop mediates DCL processing of crRNA multi-guides, thereby releasing the individual guide sequences to somehow associate with AGO. Another hypothesis is that complementary binding of crRNA-mRNA alone triggers SMRRT silencing, and intervening sequences between the guides does not impact the phenomena.

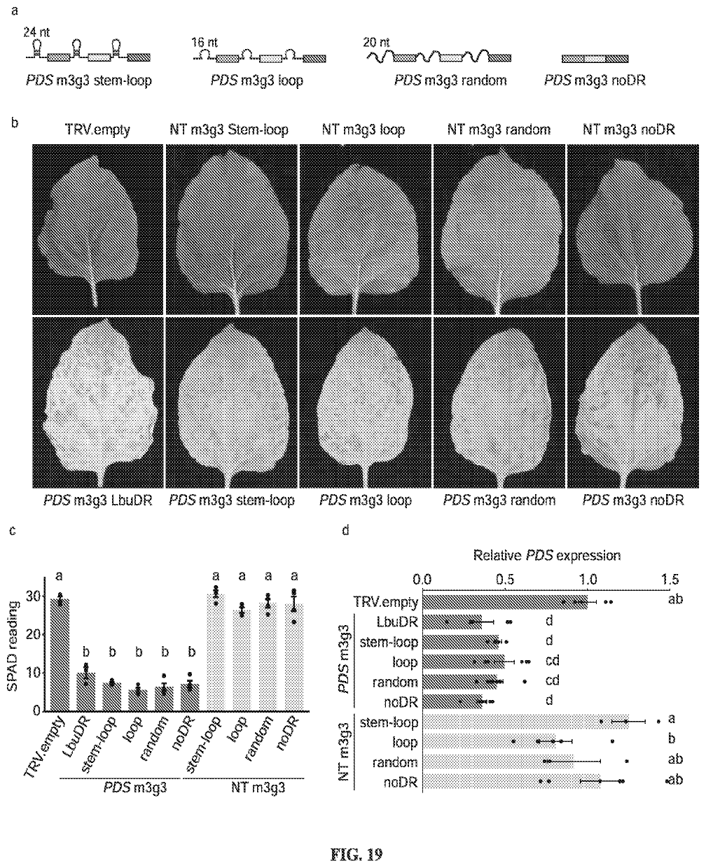

To test these hypotheses and determine if the Cas13a DR sequence, or the resulting stem-loop structure are required for the induction of SMRRT silencing, we tested modified PDS m3g3 construct designs (FIG. 19a). We evaluated four PDS m3g3 construct designs where the 5′ guide sequence was varied (FIG. 19a). Expressing the modified fragments using TRV delivery in N. benthamiana, we found that the intervening 5′ sequence for PDS m3g3 did not affect visible photobleaching (FIG. 19b). That is, replacing the crRNA DR with either an Arabidopsis miR170 stem-loop, an Arabidopsis miR170 lacking 4 nt stem (i.e., loop only), a random 20 nt sequence, or no DR all (directly connected) elicited a clearly visible photobleaching phenotype (FIG. 19b). Each of these modified constructs were also tested with a non-target (NT)-guide control, and no design elicited photobleaching alone (FIG. 19b). Chlorophyll content in plants expressing PDS m3g3 LbuDR, stem-loop, loop, random, and no DR was reduced by 65-80%, compared to the TRV_{empty} control, and no significant reduction was observed for the guide design controls expressing the NT m3g3 crRNA (FIG. 19c). The visible photobleaching pattern was supported by PCR transcript quantification, where we observed 35% to 50% reduced PDS transcript levels compared to the TRV_{empty} control (FIG. 19d). Together, these results show m3g3 5′ guide sequence does not require a DR or any specific sequence for GIGS initiation.

Guide Order of crRNA in PDS Multi-Guide Construct Did not Alter Silencing Strength

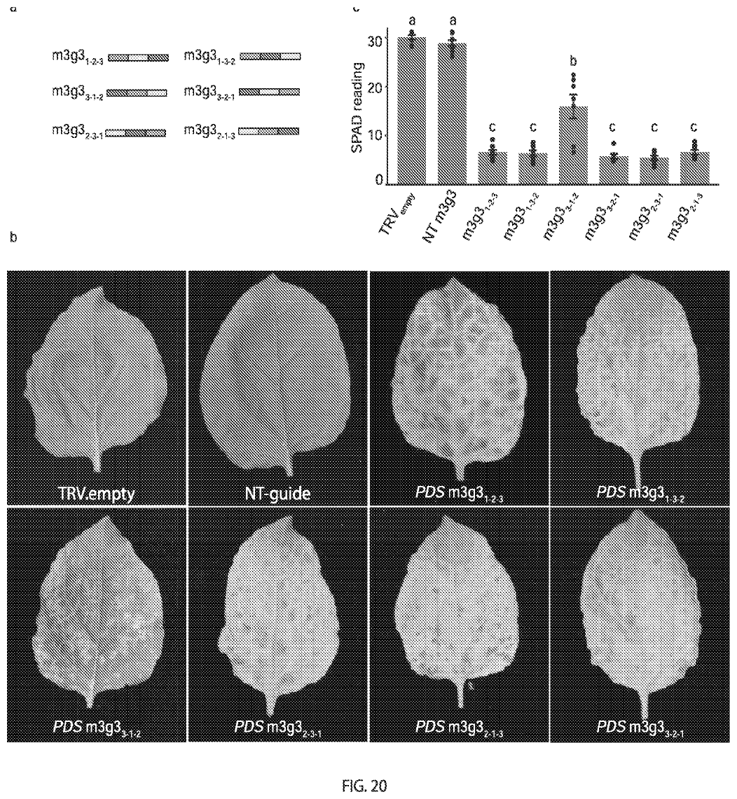

PDS m3g3 expressed as a single RNA molecule elicited a visible photobleaching response (FIG. 17). When a m3g3 crRNA is expressed as single RNA molecule, it is hypothesized that all three crRNA could hybridize with the targeted mRNA to change its confirmation or only one crRNA hybridizes with RNA while the other two adjacent crRNA in the m3g3 remain unattached. Given our limited knowledge on how many crRNA in a multi-guide hybridizes with RNA or in which order they hybridize with the target RNA, we tested if the guide order in a multi-guide crRNA influences RNA silencing activity. We hypothesized that guide order of individual crRNAs in the m3g3 crRNA does not affect DCL-processing activity. We evaluated six TRV infectious clones generated by rearranging three PDS m3g3 crRNA in six different combinations based on their position in the multi-guide (FIG. 20a). Five of the six crRNA treatments elicited a similar mosaic photobleaching pattern in the lower leaves, and one crRNA construct elicited only a partial photobleaching (FIG. 20b). No photobleaching was observed in plants infected with the TRV_{empty} control. Consistent with the visible photobleaching patterns, we observed similar levels of chlorophyll reduction with five of the six rearranged PDS guide crRNA constructs, and only slightly less for the sixth construct (FIG. 20c).

Increasing crRNA Fragment Number in a Multi-Guide Produced Stronger Photobleaching

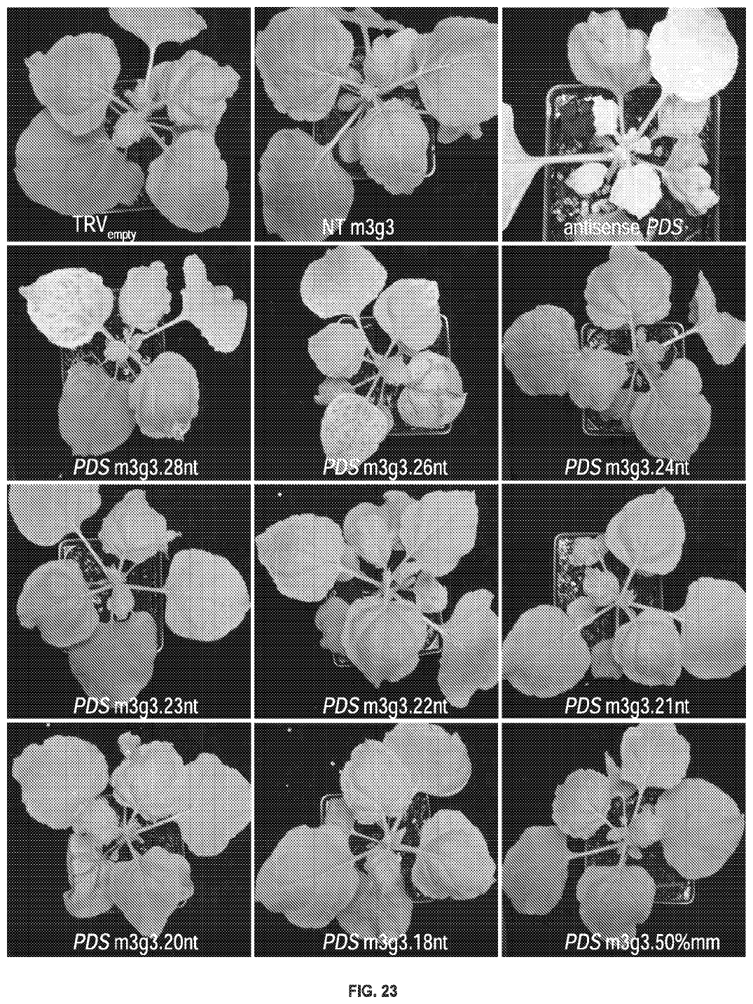

Previous research identified antisense fragments in the range of ˜200 to ˜1300 nt silenced RNA more effectively than short inserts (for RNAi). Supporting these findings, the visible photobleaching phenotype elicited by a 397 nt antisense PDS fragment was completely white with almost no chlorophyll in the lower leaves (FIG. 18b, 19c, and FIG. 23). In contrast, PDS m3g3 crRNA elicited a visible photobleaching phenotype in the lower leaves that was more mosaic, less white, and leads to a lesser reduction in chlorophyll content (FIG. 18c). In the newly emerged leaves, antisense PDS caused complete photobleaching while PDS m3g3 elicited no visible photobleaching phenotype (FIG. 23). One possibility for lesser reduction in chlorophyll content with PDS m3g3 could be attributed to differences in the length of the RNA targeted by the crRNA fragments and the and antisense PDS fragments. We hypothesized that SMRFs targeting more locations on an RNA causes greater RNA degradation.

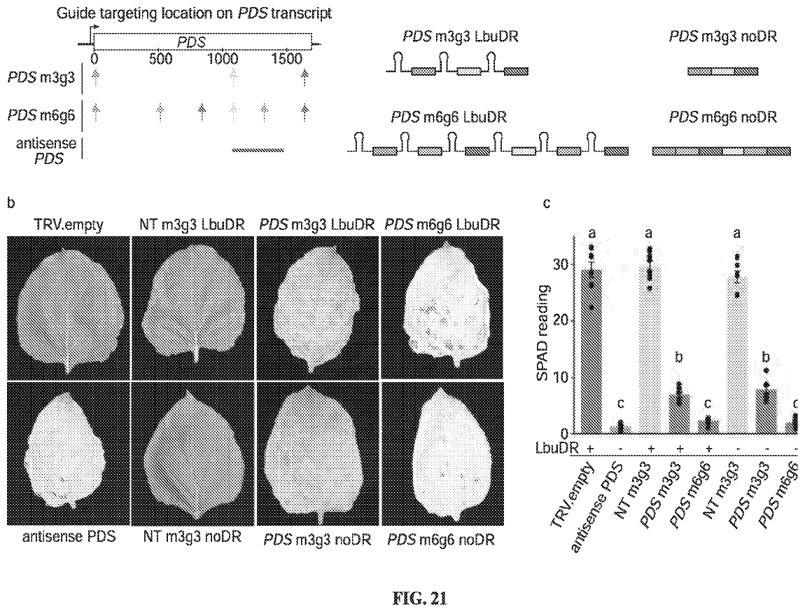

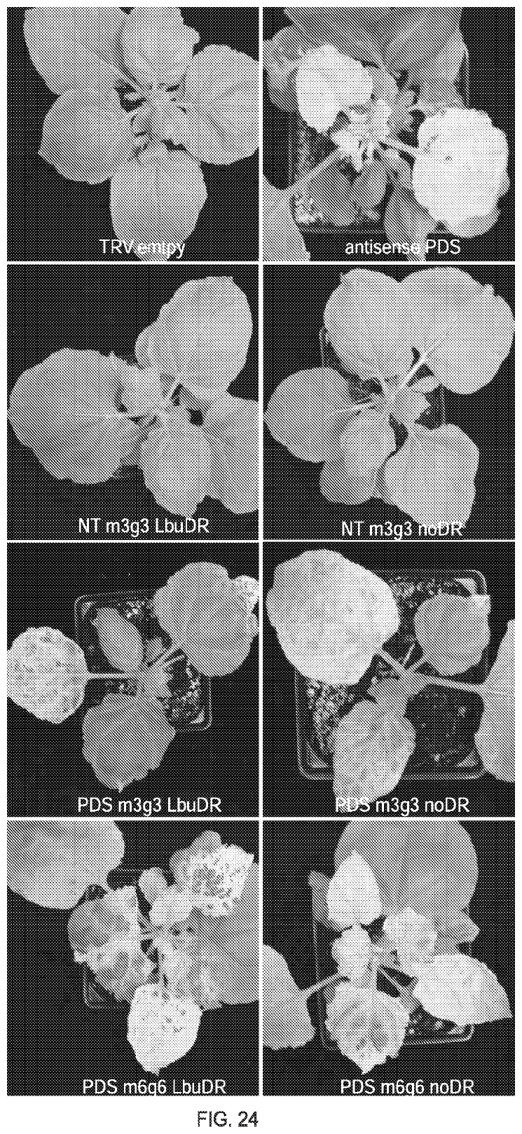

To test this hypothesis, we generated a PDS m6g6 (six guides targeting six different regions of the PDS transcript) by adding three additional crRNA fragments to the PDS m3g3 construct (FIG. 21a). Two weeks after systemic TRV movement, PDS m6g6 crRNA SMRFs elicited a completely clear photobleaching phenotype in the lower and newly emerged leaves (FIG. 21b and FIG. 24). The visual photobleaching phenotype elicited with PDS m6g6 crRNA SMRFs was same as the photobleaching pattern caused by the conventional RNAi antisense PDS fragment. No visible differences were observed in the photobleaching phenotype elicited by PDS m6g6 constructs containing LbuDR or noDR sequence between the guides. Expressing PDS m3g3 crRNA produced a mosaic photobleaching phenotype in lower leaves while the top leaves remained green, and the TRV_{empty} control plants lacked any photobleaching appearance. Supporting the visual photobleaching patterns, ˜95% chlorophyll content reduction was elicited with PDS m6g6 with or without DR and antisense PDS, and PDS m3g3 caused a 76% reduction compared to TRV_{empty} (FIG. 21c). We observed a significant difference in chlorophyll content levels between PDS m6g6 and PDS m3g3 crRNA. Collectively, these results demonstrate the strength of photobleaching phenotype, elicited by PDS silencing, increased with an increase in the number of guide crRNA in the PDS multi-guide.

### Increasing Guide Dosage of Single-Guides Produced Stronger Silencing

RNAi off-targeting activity stems from sRNA generated through DCL-mediated cleavage of dsRNA and additional sRNA generation by RdRP. Previously, we reported the antisense PDS fragment caused more sRNA production against the PIS transcript compared to a lesser and almost no sRNA elicited by PDS m3g3 and single-guide respectively. We observed the fewer sRNA generated with PDS m3g3 were against the PDS RNA in the intervening space between the three PDS guides. In contrast, s-guide targeting only one region on the PDS RNA caused no silencing but also did not produce any sRNA. One possible explanation for reduced PDS silencing with PDS m3g3 and s-guides results from their fewer sRNA generation compared to the antisense PDS fragment. Based on these findings, we hypothesized the SMRRT silencing construct design that elicits strong RNA degradation while generating fewer sRNA can be used as an RNAi alternative for crop improvement. Our previous SMRRT silencing construct design, PDS m6g6 crRNA elicited a strong visible photobleaching pattern (FIG. 21b), like antisense PDS, but targeting six different RNA regions could lead to an increase in sRNA levels in the intervening space between PDS m6g6 crRNA. Another possible SMRRT silencing construct design for effectively silencing PDS RNA while synthesizing fewer sRNA involves increasing the fragment dosage of s-guides.

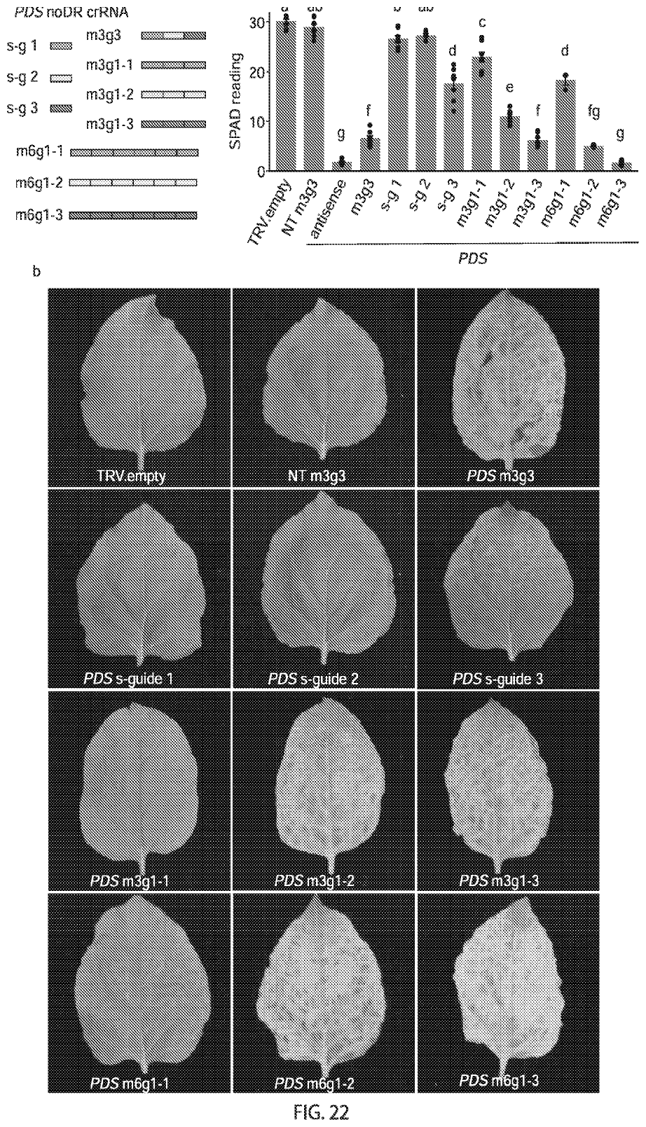

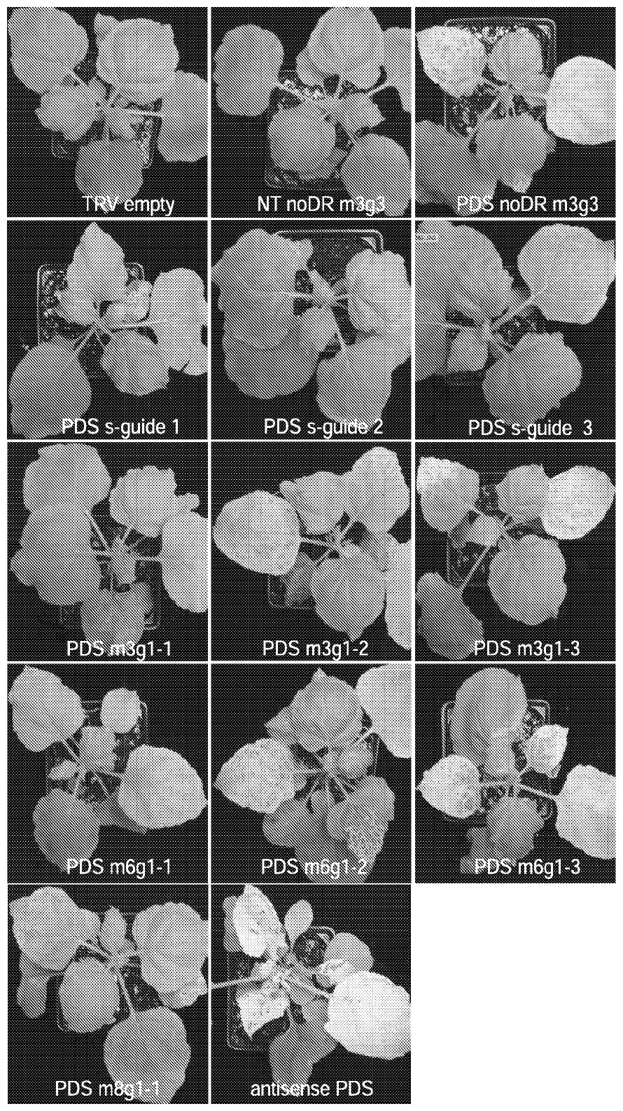

We tested the potential of increased s-guides dosage to induce strong photobleaching by assembling PDS m3g1 (3 repeat fragments targeting the same RNA, PDS m3g1-1, -2, and -3) and PDS m6g1 (6 repeat fragments targeting the same RNA, PDS m6g1-1, -2, and -3) TRV constructs (FIG. 22a). Expression of PDS m3g1-3 construct elicited a mosaic, less white, and lower reduction in chlorophyll content in the lower leaves while the top leaves remained green (FIG. 22b, 22c, and FIG. 25). Photobleaching elicited by PDS m3g1-2 was weaker compared to PDS m3g1-3, and PDS m3g1-1 crRNA caused no visible photobleaching. The photobleaching pattern elicited with PDS m3g1-3 was same as the phenotype caused by PDS m3g3. Interestingly, the three PDS m6g1 crRNA treatments elicited an increased photobleaching phenotype and greater chlorophyll reduction compared to PDS m3g1 crRNA (FIG. 22b, 22c, and FIG. 25). Comparing plants infiltrated with PDS m6g1-3 with antisense PDS revealed similar photobleaching pattern in the lower leaves, whereas the newly emerged leaves with PDS m6g1-3 crRNA remained green and antisense PDS fragment elicited a clear photobleaching phenotype (FIG. 25). Among the three PDS s-guides tested, only PDS s-guide 3 caused partial photobleaching and reduced 40% chlorophyll content compared to TRV_{empty} control. The NT m3g3 and TRV_{empty} controls lacked any photobleaching phenotype, as expected. Supporting the visible photobleaching phenotype in the lower leaves, chlorophyll reduction with antisense PDS and PDS m6g1-3 was the highest with 94% lower chlorophyll, ˜80% chlorophyll reduction with PDS m6g1-2, PDS m3g1-3, and PDS m3g3, and weak chlorophyll reduction with PDS m3g1-1 and m6g1-1 crRNA compared to the TRV_{empty} control (FIG. 21c). Combined together, these results suggest that silencing elicited by increased fragment dosage is dependent on the RNA region targeted, and increased guide dosage can elicit stronger gene silencing with a possibility of generating fewer sRNA and reduced off-targeting effects.

Increasing Guide Dosage of s-Guides Improved Silencing with Less Small RNA Generation

We observed a clear photobleaching phenotype and greater chlorophyll reduction with PDS m6g1 crRNA. Despite all our promising observations on improved GIGS guide design with TRV systemic infections in N. benthamiana, one possibility for the increased visible photobleaching phenotype elicited by increasing guide dosage could be an artefact of small RNA generated against the viruses.

### Materials and Methods

### Plant Materials

Nicotiana benthamiana plants were grown and maintained at 22° C. under 16 h light and 8 h dark cycles at 70% relative humidity. Seeds were germinated on Berger BM1 peat moss and vermiculite mix (Hummert International, Earth City, MO). One-week after emergence, N. benthamiana seedlings were transplanted into individual pots. Two-week-old seedlings were used for launching TRV infectious clones using Agrobacterium infiltration. The infiltrated plants were maintained in the growth chambers under optimal growing conditions for two weeks.

Constructing a pTRV2 Vector with AarI Restriction Site

Tobacco rattle virus (TRV) infectious clone, a generous gift from Dr. James Carrington, was used for systemic delivery of PDS and NT multi.guide crRNA. As pTRV2 vector contained a BsaI restriction site, we assembled PEBV (pea early-browning virus) promoter followed by a AarI type-II endonuclease restriction site. Overlapping oligos of PEBV promoter, 37 bp LbuCas13a direct repeat (LbuDR) or without LbuDR (noDR), with AarI restriction site were ordered from IDT (Integrated DNA Technologies, Inc., Coralville, IA). Oligos (1 ul of 100 nm concentration) were pooled and annealed using NEB T4 Polynucleotide Kinase (New England Biolabs, Ipswich, MA). pTRV2 backbone was digested using EcoRI and MluI restriction enzymes, gel electrophoresis was conducted on a 2% agarose gel to resolve the digested fragments. pTRV2 digested backbone was purified using Promega Wizard SV Gel and PCR Clean-Up System (Promega, Madison, WI). The assembled PEBV promoter with EcoRI and MluI overhangs was ligated overnight with the digested pTRV2 backbone using T4 DNA ligase (Promega Corporation, Madison, WI). Ligated pTRV2 was transformed into E. coli and selected on LB media with Kanamycin (50 μg/mL). PEBV promoter sequence was confirmed by Sanger sequencing (Genewiz, South Plainfield, NJ).

### Constructing TRV Infectious Clones

Reduced Multi.Guide crRNA Length

To generate PDS m.guide with reduced guide length, nucleotides were removed from the 3′ end for all three single guides (FIG. 17b, FIG. 23). pTRV2 backbone containing PEBV with LbuDR or noDR and AarI restriction sites were digested using AarI for 4 h. pTRV2 backbone after restriction digestion was purified to remove the fragment between two AarI restriction sites using Promega Wizard SV Gel and PCR Clean-Up System. Golden gate cloning was used to assemble PEBV-AarI digested backbone and reduced PDS m.guide crRNA with 4 bp overhangs, and transformed into E. coli for Kanamycin selection. Colony PCR was conducted using PEBV forward and PDS s.guide3 reverse primers to identify positive clones. Sequence of reduced PDS multi.guide crRNA was confirmed using Sanger sequencing with a PEBV promoter primer. Confirmed plasmid was extracted from E. coli Top10 competent cells and transformed into Agrobacterium GV3101 competent cells. Selection for positive transformants was conducted on

Modified DR Between erRNA

For LbuCas13a-mediated gene silencing, m.guide crRNA consisted of three 28nt s.guides and a direct repeat of 37 bp at the 5′ end of each guide (FIG. 17a). As GIGS does not require Cas13a protein processing of a m-guide crRNA into individual crRNA, we tested the flexibility of sequence required at the 5′ end of s.guides for GIGS. We tested PDS m.guide with four constructs (a) NoDR: no direct repeat between three s.guide crRNA, (b) Stem-loop: replaced LbuDR with Arabidopsis 24nt miR170 stemloop, (c) Loop: Arabidopsis miR170 stemloop without stem (16nt), and (d) Random: a 20nt randomly generated nucleotide sequence. Luciferase m.guide with NoDR, Stem-loop, Loop, and Random sequences were designed for including as nontarget (NT) controls. PDS and NT m.guide oligos were ordered from IDT, annealed using T4PNK ligase, cloned into pTRV2-PEBV backbone, and selected using Kanamycin. Sanger sequence was conducted to confirm PDS and NT m.guide crRNA.

Constructing Reduced PDS Guides with and without LbuCas13a

pENTR vector backbone was digested with BsaI restriction enzyme and PDS multi.guide annealed oligos were ligated using T4 DNA ligase. In pGWB413::35SLbuCas13a vector, 35S promoter was used to drive the expression of LbuCas13a.

### Measuring Chlorophyll Content

SPAD Chlorophyll Meter which instantly measures chlorophyll content or “greenness” of plants.

### Example 3

The prior work exemplified in three plant species (Nicotiana benthamiana and Solanum lycopersicum, and Arabidopsis) is extended to a soybean (Glycine max). SMRRT silencing constructs were designed for silencing enhanced green fluorescent protein (EGFP) RNA in soybean hairy roots that were constitutively expressing the fluorescent protein.

Evaluating increased guide dosage in soybean hairy roots against enhanced green fluorescent protein revealed that increased guide dosage confers strong RNA silencing in soybean. Small RNA sequencing from soybean hairy roots showed fewer small RNA production with increased guide dosage and fewer off-target effects compared to RNAi. Our results demonstrate the potential of improved SMRRT silencing for transcriptome engineering to bypass pleiotropic effects in crop improvement.

TABLE
Summary of optimized guide design in soybean hairy roots

| | # of hairy | # of roots showing |
|---|---|---|
| | roots | GFP reduction |
| Rep 1 | | |
| Luc-Control | 7 | 1 |
| eGFP.antisense | 7 | 3 |
| eGFP.m3g3 | 7 | 5 |
| eGFP.m6g1.1 | 5 | 1 + 1(?) |
| eGFP.m6g1.2 | 7 | 3 |
| eGFP.m6g1.3 | 7 | 1 |
| Rep 2 | | |
| Luc-Control | 8 | 1 |
| eGFP.antisense | 8 | 3 |
| eGFP.m3g3 | 8 | 4 |
| eGFP.m6g1.1 | 8 | 3 |
| eGFP.m6g1.2 | 8 | 3 |
| eGFP.m6g1.3 | 8 | 2 |

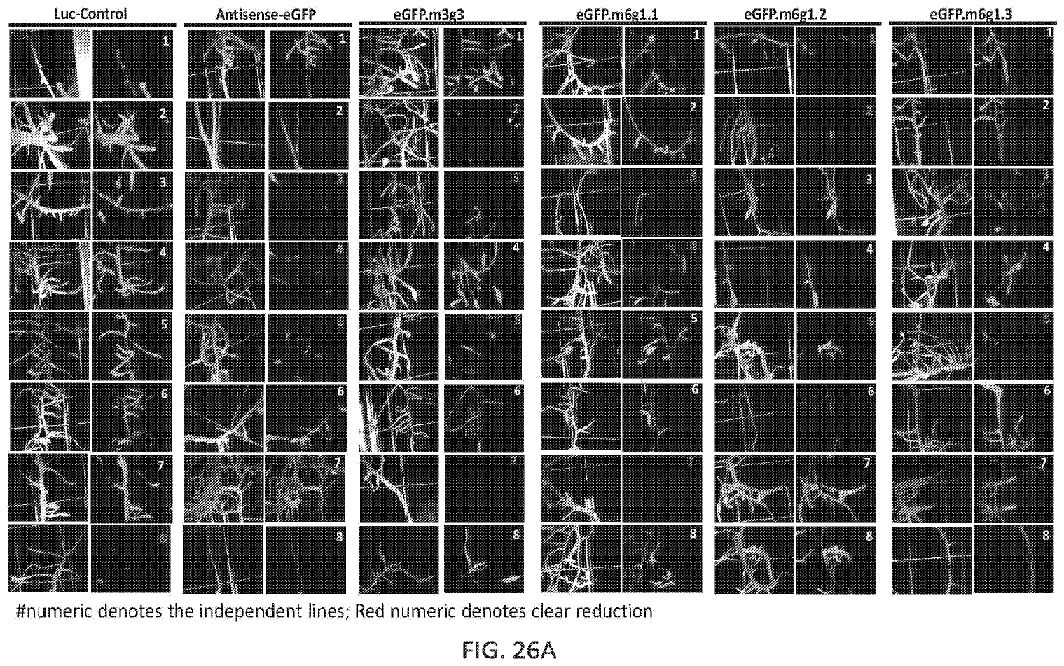

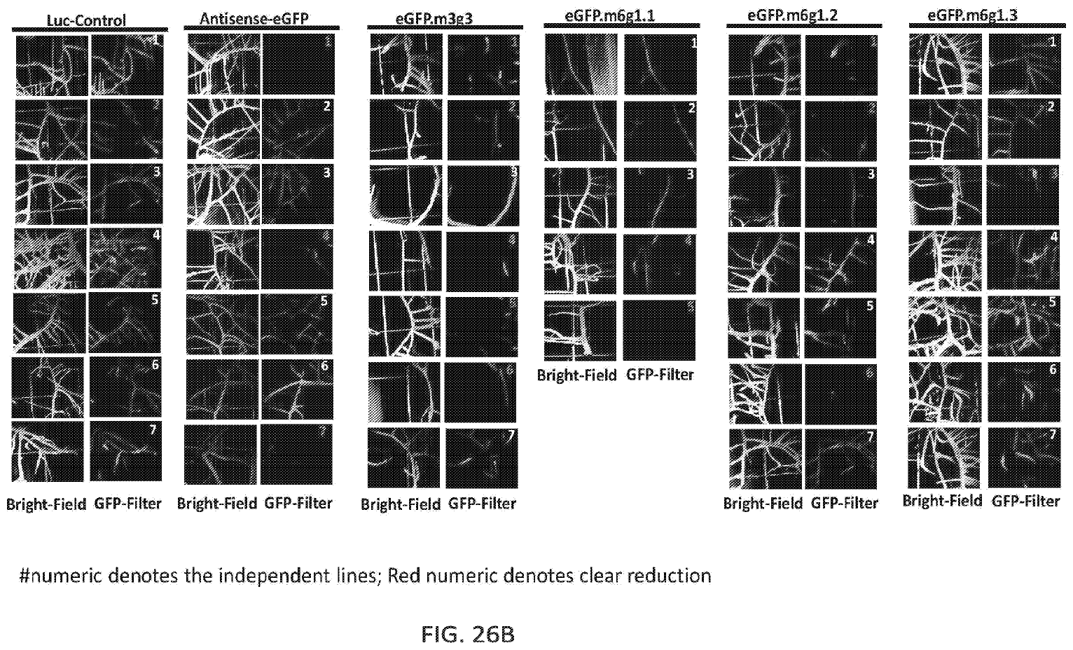

The results are shown in FIG. 26A and FIG. 26B.

1. Short multi-repeat RNA targeting constructs for manipulating one or more RNA targets, said constructs comprising two or more distinct guide nucleotide sequences that are complementary to one or more RNA targets, wherein each guide nucleotide sequence consists of a single-stranded antisense nucleotide fragment of 100 nt or less.

2. The short multi-repeat RNA targeting constructs of claim 1, wherein each guide nucleotide sequence consists of a single-stranded antisense nucleotide fragment of 15 nt to 100 nt.

3. The short multi-repeat RNA targeting constructs of claim 1, wherein each guide nucleotide sequence consists of a single-stranded antisense nucleotide fragment of 80 nt or less.

4. The short multi-repeat RNA targeting constructs of claim 1, wherein each guide nucleotide sequence consists of a single-stranded antisense nucleotide fragment of 50 nt or less.

5. The short multi-repeat RNA targeting constructs of claim 1, wherein each guide nucleotide sequence consists of a single-stranded antisense nucleotide fragment of 22 nt to 30 nt.

6. The short multi-repeat RNA targeting constructs of claim 1, wherein said guide nucleotide sequences are guide RNA (crRNA) or DNA encoding for the guide RNA, which are capable of binding or hybridizing to the RNA target.

7. The short multi-repeat RNA targeting constructs of claim 1, wherein the RNA target is a coding sequence, preferably mRNA or pre-mRNA, or a non-coding sequence, preferably ncRNA, IncRNA, tRNA, or rRNA.

8. The short multi-repeat RNA targeting constructs of claim 1, wherein said guide nucleotide sequences each bind to a different region of the same RNA target.

9.-10. (canceled)

11. The short multi-repeat RNA targeting constructs of claim 1, wherein said guide nucleotide sequences each bind to a different RNA target.

12. (canceled)

13. The short multi-repeat RNA targeting constructs of claim 1, wherein said constructs are comprised within a single expression cassette, wherein each of said two or more distinct guide nucleotide sequences are:
directly linked on said expression cassette; or
indirectly linked, such that each guide nucleotide sequence is separated by an intervening sequence on said expression cassette.

14.-16. (canceled)

17. The short multi-repeat RNA targeting constructs of claim 13, wherein said single expression cassette is a homogenous multimer wherein each of said two or more distinct guide nucleotide sequences consists of the same sequence and has complementarity with the same region of the same RNA target or complementarity with a homologous region of a different RNA target.

18. The short multi-repeat RNA targeting constructs of claim 17, wherein said homogenous multimer comprises up to eight repeats of the same guide nucleotide sequence.

19. The short multi-repeat RNA targeting constructs of claim 13, wherein said single expression cassette is a heterogenous multimer wherein each of said two or more distinct guide nucleotide sequences consists of different sequences having complementarity with different RNA targets or different regions of the same RNA target.

20. The short multi-repeat RNA targeting constructs of claim 13, wherein said constructs are located on the expression cassette adjacent at least one regulatory element.

21. The short multi-repeat RNA targeting constructs of claim 1, wherein said constructs are comprised on respective separate expression cassettes, wherein said constructs are configured for co-expression.

22. The short multi-repeat RNA targeting constructs of claim 1, said constructs being free of nuclease-encoding nucleotide sequences, or nuclease protein.

23. (canceled)

24. The short multi-repeat RNA targeting constructs of claim 1, said constructs comprising three or more of said distinct guide nucleotide sequences.

25. A composition for manipulation of RNA targets, said composition comprising a plurality of short multi-repeat RNA targeting constructs according to claim 1 dispersed in a carrier or vehicle.

26. A method of modifying an RNA target, said method comprising delivering to said RNA target a short multi-repeat RNA targeting construct according to claim 1.

27.-37. (canceled)

38. A method of gene silencing comprising delivering to a cell, tissue, organ, or organism, a short multi-repeat RNA targeting construct according to claim 1, wherein at least one of said two or more distinct guide nucleotide sequences is complementary to an RNA target associated with said gene.

39. A method of manipulating a plant characteristic, said method comprising delivering to a plant cell, tissue, or plant a short multi-repeat RNA targeting construct according to claim 1, wherein at least one of said two or more distinct guide nucleotide sequences is complementary to an RNA target associated with a gene encoding for said plant characteristic.

40.-42. (canceled)

43. A seed, seedling, or progeny of a plant produced according to the method of claim 39, wherein said seed, seedling, or progeny expresses said short multi-repeat RNA targeting construct.

44.-45. (canceled)


SEQUENCE LISTING (76 sequences)

SEQ ID NO: 1 - DNA, 3561 bp, Leptotrichia bucallis
atgtacccgtatgacgtgccggattatgcgtacccgtacgacgttccggactatgcgtacccgtatgatgttccggattatgcgaaagtgaccaaggttggtggcatcagccacaagaaatacaccagcgaaggtcgtctggttaaaagcgagagcgaggaaaaccgtaccgacgagcgtctgagcgcgctgctgaacatgcgtctggatatgtacattaagaacccgagcagcaccgaaaccaaagagaaccagaagcgtatcggtaaactgaagaaattctttagcaacaaaatggtgtatctgaaggacaacaccctgagcctgaaaaacggcaagaaagaaaacattgaccgtgaatacagcgagaccgatatcctggagagcgacgtgcgtgataagaaaaacttcgcggttctgaagaaaatctacctgaacgaaaacgtgaacagcgaggaactggaggttttccgtaacgatattaagaaaaagctgaacaaaatcaacagcctgaagtacagctttgagaagaacaaggcgaactaccaaaaaattaacgagaacaacatcgaaaaggttgagggcaagagcaagcgtaacatcatctacgactactatcgtgaaagcgcgaaacgtgatgcgtatgtgagcaacgtgaaagaggcgttcgacaaactgtacaaggaagaggacattgcgaaactggtgctggaaatcgagaacctgaccaagctggaaaaatacaagatccgtgaattctatcacgagatcattggccgtaagaacgacaaggagaactttgcgaagatcatctacgaggaaattcagaacgttaacaacatgaaagaactgatcgagaaggtgccggatatgagcgagctgaaaaagagccaagtgttctacaagtactatctggacaaagaggagctgaacgataaaaacattaagtatgcgttctgccactttgttgaaatcgagatgagccagctgctgaaaaactacgtgtataagcgtctgagcaacattagcaacgacaaaatcaagcgtatcttcgaataccaaaacctgaaaaagctgatcgagaacaagctgctgaacaaactggatacctacgtgcgtaactgcggtaaatataactactatctgcaggacggcgaaattgcgaccagcgatttcatcgcgcgtaaccgtcaaaacgaggcgtttctgcgtaacatcattggtgtgagcagcgttgcgtacttcagcctgcgtaacattctggaaaccgagaacgaaaacgacatcaccggtcgtatgcgtggcaaaaccgttaagaacaacaaaggcgaggaaaaatacgtgagcggcgaagttgataagatctataacgagaacaaaaagaacgaagtgaaggagaacctgaaaatgttctacagctatgactttaacatggataacaagaacgagattgaagacttctttgcgaacatcgatgaagcgatcagcagcattcgtcacggtattgttcacttcaacctggagctggaaggcaaagacatcttcgcgtttaagaacattgcgccgagcgagatcagcaaaaagatgtttcagaacgaaattaacgagaaaaagctgaaactgaagatcttccgtcaactgaacagcgcgaacgtgtttcgttacctggaaaagtataagatcctgaactacctgaagcgtacccgtttcgagtttgtgaacaagaacatcccgttcgttccgagctttaccaaactgtatagccgtattgacgatctgaagaacagcctgggtatctactggaagaccccgaaaaccaacgacgataacaaaaccaaggaaatcattgacgcgcagatctacctgctgaaaaacatctactatggtgaattcctgaactactttatgagcaacaacggcaacttctttgagatcagcaaagaaatcattgagctgaacaagaacgacaaacgtaacctgaagaccggcttctataagctgcagaagttcgaagatatccaagagaagatcccgaaagagtacctggcgaacattcagagcctgtatatgatcaacgcgggtaaccaagacgaggaagagaaggacacctacatcgatttcattcaaaagatcttcctgaaaggctttatgacctacctggcgaacaacggtcgtctgagcctgatctatattggcagcgacgaagagaccaacaccagcctggcggagaagaagcaggagttcgataagttcctgaaaaagtacgaacaaaacaacaacatcaagattccgtatgaaatcaacgagttcctgcgtgaaattaaactgggtaacatcctgaagtacaccgagcgtctgaacatgttttatctgatcctgaaactgctgaaccacaaggaactgaccaacctgaaaggcagcctggagaagtaccagagcgcgaacaaagaagaggcgttcagcgaccaactggagctgattaacctgctgaacctggataacaaccgtgttaccgaagactttgagctggaagcggatgagatcggtaaattcctggactttaacggcaacaaagtgaaggataacaaagaactgaaaaagttcgacaccaacaagatctactttgatggcgagaacatcattaagcaccgtgcgttctacaacattaaaaagtatggtatgctgaacctgctggagaagatcgcggacaaagcgggctacaagatcagcattgaagagctgaaaaagtatagcaacaaaaagaacgagatcgaaaagaaccacaaaatgcaggaaaacctgcaccgtaaatatgcgcgtccgcgtaaggatgagaaattcaccgacgaagattacgagagctataagcaggcgattgaaaacatcgaagagtacacccacctgaaaaacaaggttgagttcaacgagctgaacctgctgcaaggtctgctgctgcgtattctgcaccgtctggtgggctacaccagcatctgggagcgtgacctgcgtttccgtctgaagggtgaatttccggagaaccagtacatcgaagagatcttcaacttcgaaaacaaaaagaacgtgaaatacaagggtggccagatcgttgagaagtacatcaaattctataaggaactgcaccaaaacgacgaggttaaaattaacaagtacagcagcgcgaacatcaaagtgctgaagcaagagaagaaggatctgtacattcgtaactatatcgcgcactttaactacattccgcacgcggagatcagcctgctggaagttctggagaacctgcgtaaactgctgagctatgaccgtaaactgaagaacgcggttatgaaaagcgtggttgatattctgaaggagtacggcttcgtggcgacctttaagatcggtgcggacaaaaagatcggcattcagaccctggagagcgaaaaaatcgttcacctgaagaacctgaaaaagaaaaagctgatgaccgatcgtaacagcgaagagctgtgcaaactggtgaagattatgttcgaatacaagatggaggagaaaaagagcgagaactaa

SEQ ID NO: 2 - DNA, 4431 bp, Lachnospiraceae bacterium
atggactacaaggaccacgacggtgactacaaggaccacgacatcgactacaaggacgacgacgacaagaaaatcagtaaagttcgtgaggaaaatcgaggtgccaagcttaccgttaacgctaaaactgcagtcgtttccgagaacagatcacaggagggcattttatacaacgatccctctagatatgggaaaagtaggaaaaacgacgaggacagagacaggtacattgagtcaaggctgaagagtagtggaaaattatatagaattttcaatgaggacaaaaataagagagaaactgacgaactccaatggtttttgagcgaaattgtgaaaaagatcaaccgtcgtaacggccttgttcttagtgacatgctttcagtggatgatcgtgctttcgaaaaagccttcgaaaaatacgctgagttgtcttacacgaacagacgaaataaagtcagtggttctcccgcatttgaaacatgtggggtcgacgcagctactgcagagcgactgaaggggataataagtgagacaaactttattaatcgtattaaaaataacatagacaataaggtctcagaagacattatagatcgtataatagcaaagtatttgaagaaaagcctttgccgtgaaagggttaagcgtggattaaagaagctcttgatgaatgcatttgacttaccgtacagtgatcccgacattgacgtacaacgagattttatagactatgtccttgaagatttttatcacgtgagagcaaagagtcaagttagtagaagtattaagaatatgaatatgccagtacagcccgagggggacgggaaattcgctataaccgtctccaagggcgggactgaatcagggaacaagcgaagcgcagagaaggaggcttttaagaaatttttatcagattacgcatctttagatgagagagtgcgagatgacatgttacgtcgaatgagaaggcttgtcgtactttatttttacggttccgacgattctaaattgtctgatgttaatgaaaaattcgatgtttgggaagatcatgctgctcgtagggtggataacagggagttcataaagcttccgctggaaaacaaactggccaacgggaagaccgataaagatgccgagaggattcgaaagaatacagtaaaggaactgtacagaaaccaaaatattgggtgttaccgacaagcagtcaaggcagtggaagaggataataacgggcgatattttgacgacaagatgctcaatatgttttttattcaccgtatcgaatatggagtcgagaaaatatacgcaaacctcaaacaggttactgaatttaaagctaggactgggtatttaagcgaaaaaatttggaaagatctcataaactacatatccataaagtatatcgcaatgggtaaagcagtctacaactatgcaatggatgaattaaatgccagcgacaagaaggagatcgagctcgggaaaatatcagaagaatatttgtctgggatcagtagtttcgattacgagcttataaaggcagaagaaatgttacagcgtgaaactgccgtctatgtggctttcgccgccaggcacctgtcatctcaaacagttgagttggattcagagaatagcgacttccttctcttgaaaccgaagggcacaatggataagaatgataagaacaaactcgcatcaaataacatcctgaattttctcaaagacaaagaaaccctcagggacactattttacaatactttggtgggcacagcctttggaccgattttccctttgacaaatatctggcagggggtaaggatgacgttgattttctcaccgacttgaaagacgtgatctacagcatgcgaaacgacagcttccattatgctacagaaaaccataataacggcaagtggaacaaagagcttatctccgctatgttcgaacatgaaactgaaagaatgacagtagtaatgaaggataaattttactctaacaacttaccgatgttttacaagaacgatgatctgaagaaactcttaattgacctttacaaggacaacgtcgagcgagcctcacaggtcccctcctttaataaggtgtttgtccgaaagaactttcccgccttagtccgtgacaaggacaatcttgggatagagctggatctgaaggcagatgcagataagggcgaaaacgagctgaagttttataacgctctttattatatgttcaaggaaatttactataatgcattcttgaacgataaaaatgtgagggagaggttcatcactaaggccaccaaagtagcagacaattacgaccgaaacaaggagaggaatcttaaagaccgaattaaaagtgctgggagtgatgaaaaaaagaaacttcgtgagcagttgcagaattacatagcagaaaacgatttcggacagaggattaagaacatagtacaggttaacccagattataccttggcacaaatatgtcagcttataatgactgagtacaaccaacaaaacaacgggtgtatgcagaaaaagagtgcagccagaaaagacattaacaaggattcataccagcactataagatgcttctgttggtaaacctgagaaaagcattcctggaattcatcaaagaaaactatgcatttgtgttgaagccgtataagcacgatctctgtgacaaagccgattttgttcccgatttcgcaaaatatgtgaaaccatatgcaggcctcatttcacgagtcgccgggagcagtgagctccaaaagtggtatattgtatcacgtttcttgtccccggcacaagccaaccacatgttgggattcctgcatagttacaaacagtacgtatgggacatctatcgtcgtgctagtgaaactgggactgagataaatcattctatagccgaggataaaatagcaggtgtcgatattaccgacgtggatgcagtcattgaccttagcgtaaaactctgtggcacgatttcttctgaaataagtgattacttcaaggacgacgaggtttacgccgaatatatttccagctacctggatttcgagtacgatggtggtaactacaaggactctctgaacaggttttgtaattcagacgctgtaaacgatcaaaaggtagcattatattatgatggcgaacatccaaagttaaatcgaaacattattttatccaagctgtatggggagagaaggttccttgagaagattaccgacagagttagcaggtccgatatagtggagtattacaagttaaagaaagagacgagccaatatcaaactaaaggcatctttgacagtgaggacgagcaaaagaacataaaaaaattccaagaaatgaaaaatatagtagagtttagagatcttatggattatagtgagattgcagatgagctccaaggacagctgatcaactggatatatctgagagaacgtgacttgatgaacttccagcttggctaccactatgcctgtcttaacaacgactctaataaacaagccacctacgtgacacttgattaccagggtaaaaaaaacaggaagatcaatggggccatactttaccagatctgtgctatgtatattaacgggctcccactttactacgtagataaggattccagcgaatggacagttagtgacggcaaggaatcaacgggtgcaaagataggtgaattttataggtacgctaagtcatttgagaatacatccgactgttatgcttctggcttagaaatattcgagaatatatctgaacacgataacataacggagcttagaaactatatcgaacactttcgatactattcatccttcgaccgttcttttttaggaatttactccgaggtattcgaccgattttttacgtacgatctgaaatataggaaaaacgtccccacgatactctacaatatattactccaacacttcgttaacgtcaggttcgaatttgtctctggtaaaaagatgatagggatagacaaaaaggataggaaaatagctaaagagaaagagtgcgctaggattacaatcagggagaagaatggcgtatattctgagcagtttacatacaaattgaagaacggtactgtgtatgtggacgcacgagacaaacgttacctgcagtccattatcaggctgttattttacccagaaaaggtgaatatggatgagatgatcgaggtgaaggagaagaagaaaccgagcgataataataccggaaaagggtatagtaaaagggatcgtcaacaagacaggaaagaatacgataaatacaaggaaaaaaagaagaaagagggtaattttctcagcgggatgggagggaacatcaactgggatgagattaatgctcaattgaaaaacggcggcgggggttctggcggaggaggtagcggggggggggggagccctaggtaa

SEQ ID NO: 3 - DNA, 1761 bp, Nicotiana benthamiana
atgccccaaattggacttgtttctgccgttaatttgagagtccaaggtaattcagcttatctttggagctcgaggtcttcgttgggaactgaaagtcaagatgtttgcttgcaaaggaatttgttatgttttggtagtagcgactccatggggcataagttaaggattcgtactccaagtgccacgacccgaagattgacaaaggactttaatcctttaaaggtagtctgcattgattatccaagaccagagctagacaatacagttaactatttggaggcggcgttattatcatcatcgtttcgtacttcctcacgcccaactaaaccattggagattgttattgctggtgcaggtttgggtggtttgtctacagcaaaatatctggcagatgctggtcacaaaccgatattgctggaggcaagagatgtcctaggtgggaaggtagctgcatggaaagatgatgatggagattggtacgagactgggttgcacatattctttggggcttacccaaatatgcagaacctgtttggagaactagggattgatgatcggttgcagtggaaggaacattcaatgatatttgcgatgcctaacaagccaggggagttcagccgctttgattttcctgaagctcttcctgcgccattaaatggaattttggccatactaaagaacaacgaaatgcttacgtggcccgagaaagtcaaatttgctattggactcttgccagcaatgcttggagggcaatcttatgttgaagctcaagacggtttaagtgttaaggactggatgagaaagcaaggtgtgcctgatagggtgacagatgaggtgttcattgccatgtcaaaggcacttaacttcataaaccctgacgagctttcgatgcagtgcattttgattgctttgaacagatttcttcaggagaaacatggttcaaaaatggcctttttagatggtaaccctcctgagagactttgcatgccgattgtggaacatattgagtcaaaaggtggccaagtcagactaaactcacgaataaaaaagatcgagctgaatgaggatggaagtgtcaaatgttttatactgaataatggcagtacaattaaaggagatgcttttgtgtttgccactccagtggatatcttgaagcttcttttgcctgaagactggaaagagatcccatatttccaaaagttggagaagctagtgggagttcctgtgataaatgtccatatatggtttgacagaaaactgaagaacacatctgataatctgctcttcagcagaagcccgttgctcagtgtgtacgctgacatgtctgttacatgtaaggaatattacaaccccaatcagtctatgttggaattggtatttgcacccgcagaagagtggataaatcgtagtgactcagaaattattgatgctacaatgaaggaactagcgaagcttttccctgatgaaatttcggcagatcagagcaaagcaaaaatattgaagtatcatgttgtcaaaaccccaaggtctgtttataaaactgtgccaggttgtgaaccctgtcggcccttgcaaagatcccctatagagggtttttatttagctggtgactacacgaaacagaagtacttggcttcaatggaaggtgctgtcttatcaggaaagctttgtgccgaagctattgtacaggattacgagttacttcttggccggagccagaagatgttggcagaagcaagcgtagttagcatagtgaactaa

SEQ ID NO: 4 - DNA, 1752 bp, Solanum lycopersicum
atgcctcaaattggacttgtttctgctgttaacttgagagtccaaggtagttcagcttatctttggagctcgaggtcgtcttctttgggaactgaaagtcgagatggttgcttgcaaaggaattcgttatgttttgctggtagcgaatcaatgggtcataagttaaagattcgtactccccatgccacgaccagaagattggttaaggacttggggcctttaaaggtcgtatgcattgattatccaagaccagagctggacaatacagttaactatttggaggctgcatttttatcatcaacgttccgtgcttctccgcgcccaactaaaccattggagattgttattgctggtgcaggtttgggtggtttgtctacagcaaaatatttggcagatgctggtcacaaaccgatactgctggaggcaagggatgttctaggtggaaaggtagctgcatggaaagatgatgatggagattggtacgagactggtttgcatatattctttggggcttacccaaatattcagaacctgtttggagaattagggattaacgatcgattgcaatggaaggaacattcaatgatatttgcaatgccaagcaagccaggagaattcagccgctttgatttctccgaagctttacccgctcctttaaatggaattttagccatcttaaagaataacgaaatgcttacatggccagagaaagtcaaatttgcaattggactcttgccagcaatgcttggagggcaatcttatgttgaagctcaagatgggataagtgttaaggactggatgagaaagcaaggtgtgccggacagggtgacagatgaggtgttcattgctatgtcaaaggcactcaactttataaaccctgacgaactttcaatgcagtgcattttgatcgcattgaacaggtttcttcaggagaaacatggttcaaaaatggcctttttagatggtaatcctcctgagagactttgcatgccgattgttgaacacattgagtcaaaaggtggccaagtcagactgaactcacgaataaaaaagattgagctgaatgaggatggaagtgtcaagagttttatactgagtgacggtagtgcaatcgagggagatgcttttgtgtttgccgctccagtggatattttcaagcttctattgcctgaagactggaaagagattccatatttccaaaagttggagaagttagtcggagtacctgtgataaatgtacatatatggtttgacagaaaactgaagaacacatatgatcatttgctcttcagcagaagctcactgctcagtgtgtatgctgacatgtctgttacatgtaaggaatattacaaccccaatcagtctatgttggaattggtttttgcacctgcagaagagtggatatctcgcagcgactcagaaattattgatgcaacgatgaaggaactagcaacgctttttcctgatgaaatttcagcagatcaaagcaaagcaaaaatattgaagtaccatgttgtcaaaactccgaggtctgtttataaaactgtgccaggttgtgaaccctgtcggcctttacaaagatccccaatagaggggttttatttagccggtgactacacgaaacagaaatacttggcttcaatggaaggcgctgtcttatcaggaaagctttgtgctcaagctattgtacaggattatgagttacttgttggacgtagccaaaagaagttgtcggaagcaagcgtagtttag

SEQ ID NO: 5 - DNA, 1042 bp, Arabidopsis thaliana
atggataattcagctccagattcgttatccagatcggaaaccgccgtcacatacgactcaccatatccactctacgccatggctttctcttctctccgctcatcctccggtcacagaatcgccgtcggaagcttcctcgaagattacaacaaccgcatcgacattctctctttcgattccgattcaatgaccgttaagcctctcccgaatctctccttcgagcatccttatcctccaacaaagctaatgttcagtcctccttctctccgtcgtccttcctccggagatctcctcgcttcctccggcgatttcctccgtctttgggaaattaacgaagattcatcaaccgtcgagccaatctcggttctcaacaacagcaaaacgagcgagttttgtgcgccgttgacttccttcgattggaacgatgtagagccgaaacgtctcggaacttgtagtattgatacgagctccttagagtttgacgtgtacgatttgggatattgagaagtctgttgttgagactcagcttatagctcatgataaagaggttcatgacattgcttggggagaagctagggttttcgcatcagtctctgctgatggatccgttaggatctttgatttacgtgataaggaacattctacaatcatttacgagagtcctcagcctgatacgcctttgttaagacttgcttggaacaaacaagatcttagatatatggctacgattttgatggattctaataaggttgtgattctcgatattcgttcgccgactatgcctgttgctgagcttgaaagacatcaggctagtgtgaatgctatagcttgggcgcctcagagctgtaaacatatttgttctggtggtgatgatacacaggctcttatttgggagcttcctactgttgctggacccaatgggattgatccgatgtcggtttattcggctggttcggagattaatcagttgcagtggtcttcttcgcagcctgattggattggtattgcttttgctaacaaaatgcagctccttagagtttga

SEQ ID NO: 6 - DNA, 1350 bp, Artificial Sequence
  Synthetic RNAi PDS hairpin construct
gctggaattctagtatactaaaccaatggactttgcatgccgattgtggaacatattgagtcaaaaggtggccaagtcagactaaactcacgaataaaaaagatcgagctgaatgaggatggaagtgtcaaatgttttatactgaataatggcagtacaattaaaggagatgcttttgtgtttgccactccagtggatatcttgaagcttcttttgcctgaagactggaaagagatcccatatttccaaaagttggagaagctagtgggagttcctgtgataaatgtccatatatggtttgacagaaaactgaagaacacatctgataatctgctcttcagcagaagcccgttgctcagtgtgtacgctgacatgtctgttacatgtaaggaatattacaaccccaatcagtctatgttggaattggtatttgcacccgcagaagagtggataaatcgtagtgactcagaaattattgatgctacaatgaaggaactagcgaagcttttccctgatgaaatttcggcagtactctttctgggagtttcgaattttgttgtttctagaattcgaatttgtgattttgcggaactttgaattgatatttgtgttggttgattgtctaagtgttgttggttcttattagatttttttggatattgtagatttaatgtaaagttggtgtgagattattatttcaagcttcttttaattcaaaggcttttaaagctaatgctacagtgaaattgaatgcttagaagattgtttggaacgaaaacagattttggcatttttgtggatgatgtgttatgttgaaacgttgtctcattttcatgttgtattgtagtgccgaaatttcatcagggaaaagcttcgctagttccttcattgtagcatcaataatttctgagtcactacgatttatccactcttctgcgggtgcaaataccaattccaacatagactgattggggttgtaatattccttacatgtaacagacatgtcagcgtacacactgagcaacgggcttctgctgaagagcagattatcagatgtgttcttcagttttctgtcaaaccatatatggacatttatcacaggaactcccactagcttctccaacttttggaaatatgggatctctttccagtcttcaggcaaaagaagcttcaagatatccactggagtggcaaacacaaaagcatctcctttaattgtactgccattattcagtataaaacatttgacacttccatcctcattcagctcgatcttttttattcgtgagtttagtctgacttggccaccttttgactcaatatgttccacaatcggcatgcaaagtccat

SEQ ID NO: 7 - DNA, 10964 bp, Artificial Sequence
  Synthetic plasmid pGWB413
tttcacgcccttttaaatatccgattattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcctgcaggtcgactctagagttatcaacaagtttgtacaaaaaagctgaacgagaaacgtaaaatgatataaatatcaatatattaaattagattttgcataaaaaacagactacataatactgtaaaacacaacatatccagtcactatggcggcccctcgaggggccgcattaggcaccccaggctttacactttatgcttccggctcgtataatgtgtggattttgagttaggatccggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaagatctggatccggcttactaaaagccagataacagtatgcgtatttgcgcgctgatttttgcggtataagaatatatactgatatgtatacccgaagtatgtcaaaaagaggtgtgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggttgataacagcgggttaattaacatcttttacccatacgatgttcctgactatgcgggctatccgtatgacgtcccggactatgcaggatcctatccatatgacgttccagattacgctgctcagtgcagcgcttagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggttccggaaccaattcgtaatcatggtcatagctgtttcctgtgtgaaattgttatccgctcacaattccacacaacatacgagccggaagcataaagtgtaaagcctggggtgcctaatgagtgagctaactcacattaggctgaattaggcgcgcctatttctgaattcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtgcatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatgaattcagaaataaattcagcctaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaacct

SEQ ID NO: 8 - DNA, 6791 bp, Artificial Sequence
  Synthetic plasmid pTRV1
ataaaacatttcaatcctttgaacgcggtagaacgtgctaattggattttggtgagaacgcggtagaacgtacttatcacctacagttttattttgtttttctttttggtttaatctatccagcttagtaccgagtgggggaaagtgactggtgtgcctaaaaccttttctttgatactttgtaaaaatacatacagatacaatggcgaacggtaacttcaagttgtctcaattgctcaatgtggacgagatgtctgctgagcagaggagtcatttctttgacttgatgctgactaaacctgattgtgagatcgggcaaatgatgcaaagagttgttgttgataaagtcgatgacatgattagagaaagaaagactaaagatccagtgattgttcatgaagttctttctcagaaggaacagaacaagttgatggaaatttatcctgaattcaatatcgtgtttaaagacgacaaaaacatggttcatgggtttgcggctgctgagcgaaaactacaagctttattgcttttagatagagttcctgctctgcaagaggtggatgacatcggtggtcaatggtcgttttgggtaactagaggtgagaaaaggattcattcctgttgtccaaatctagatattcgggatgatcagagagaaatttctcgacagatatttcttactgctattggtgatcaagctagaagtggtaagagacagatgtcggagaatgagctgtggatgtatgaccaatttcgtgaaaatattgctgcgcctaacgcggttaggtgcaataatacatatcagggttgtacatgtaggggtttttctgatggtaagaagaaaggcgcgcagtatgcgatagctcttcacagcctgtatgacttcaagttgaaagacttgatggctactatggttgagaagaaaactaaagtggttcatgctgctatgctttttgctcctgaaagtatgttagtggacgaaggtccattaccttctgttgacggttactacatgaagaagaacgggaagatctatttcggttttgagaaagatccttccttttcttacattcatgactgggaagagtacaagaagtatctactggggaagccagtgagttaccaagggaatgtgttctacttcgaaccgtggcaggtgagaggagacacaatgcttttttcgatctacaggatagctggagttccgaggaggtctctatcatcgcaagagtactaccgaagaatatatatcagtagatgggaaaacatggttgttgtcccaattttcgatctggtcgaatcaacgcgagagttggtcaagaaagacctgtttgtagagaaacaattcatggacaagtgtttggattacatagctaggttatctgaccagcagctgaccataagcaatgttaaatcatacttgagttcaaataattgggtcttattcataaacggggcggccgtgaagaacaagcaaagtgtagattctcgagatttacagttgttggctcaaactttgctagtgaaggaacaagtggcgagacctgtcatgagggagttgcgtgaagcaattctgactgagacgaaacctatcacgtcattgactgatgtgctgggtttaatatcaagaaaactgtggaagcagtttgctaacaagatcgcagtcggcggattcgttggcatggttggtactctaattggattctatccaaagaaggtactaacctgggcgaaggacacaccaaatggtccagaactatgttacgagaactcgcacaaaaccaaggtgatagtatttctgagtgttgtgtatgccattggaggaatcacgcttatgcgtcgagacatccgagatggactggtgaaaaaactatgtgatatgtttgatatcaaacggggggcccatgtcttagacgttgagaatccgtgccgctattatgaaatcaacgatttctttagcagtctgtattcggcatctgagtccggtgagaccgttttaccagatttatccgaggtaaaagccaagtctgataagctattgcagcagaagaaagaaatcgctgacgagtttctaagtgcaaaattctctaactattctggcagttcggtgagaacttctccaccatcggtggtcggttcatctcgaagcggactgggtctgttgttggaagacagtaacgtgctgacccaagctagagttggagtttcaagaaaggtagacgatgaggagatcatggagcagtttctgagtggtcttattgacactgaagcagaaattgacgaggttgttccagccttttcagctgaatgtgaaagaggggaaacaagcggtacaaaggtgttgtgtaaacctttaacgccaccaggatttgagaacgtgttgccagctgtcaaacctttggtcagcaaaggaaaaacggtcaaacgtgtcgattacttccaagtgatgggaggtgagagattaccaaaaaggccggttgtcagtggagacgattctgtggacgctagaagagagtttctgtactacttagatgcggagagagtcgctcaaaatgatgaaattatgtctctgtatcgtgactattcgagaggagttattcgaactggaggtcagaattacccgcacggactgggagtgtgggatgtggagatgaagaactggtgcatacgtccagtggtcactgaacatgcttatgtgttccaaccagacaaacgtatggatgattggtcgggatacttagaagtggctgtttgggaacgaggtatgttggtcaacgacttcgcggtcgaaaggatgagtgattatgtcatagtttgcgatcagacgtatctttgcaataacaggttgatcttggacaatttaagtgccctggatctaggaccagttaactgttcttttgaattagttgacggtgtacctggttgtggtaagtcgacaatgattgtcaactcagctaatccttgtgtcgatgtggttctctctactgggagagcagcaaccgacgacttgatcgagagattcgcgagcaaaggttttccatgcaaattgaaaaggagagtgaagacggttgattcttttttgatgcattgtgttgatggttctttaaccggagacgtgttgcatttcgatgaagctctcatggcccatgctggtatggtgtacttttgcgctcagatagctggtgctaaacgatgtatctgtcaaggagatcagaatcaaatttctttcaagcctagggtatctcaagttgatttgaggttttctagtctggtcggaaagtttgacattgttacagaaaaaagagaaacttacagaagtccagcagatgtggctgccgtattgaacaagtactatactggagatgtcagaacacataacgcgactgctaattcgatgacggtgaggaagattgtgtctaaagaacaggtttctttgaagcctggtgctcagtacataactttccttcagtctgagaagaaggagttggtaaatttgttggcattgaggaaagtggcagctaaagtgagtacagtacacgagtcgcaaggagagacattcaaagatgtagtcctagtcaggacgaaacctacggatgactcaatcgctagaggtcgggagtacttaatcgtggcgttgtcgcgtcacacacaatcacttgtgtatgaaactgtgaaagaggacgatgtaagcaaagagatcagggaaagtgccgcgcttacgaaggcggctttggcaagattttttgttactgagaccgtcttatgacggtttcggtctaggtttgatgtctttagacatcatgaagggccttgcgccgttccagattcaggtacgattacggacttggagatgtggtacgacgctttgtttccgggaaattcgttaagagactcaagcctagacgggtatttggtggcaacgactgattgcaatttgcgattagacaatgttacgatcaaaagtggaaactggaaagacaagtttgctgaaaaagaaacgtttctgaaaccggttattcgtactgctatgcctgacaaaaggaagactactcagttggagagtttgttagcattgcagaaaaggaaccaagcggcacccgatctacaagaaaatgtgcacgcaacagttctaatcgaagagacgatgaagaagttgaaatctgttgtctacgatgtgggaaaaattcgggctgatcctattgtcaatagagctcaaatggagagatggtggagaaatcaaagcacagcggtacaggctaaggtagtagcagatgtgagagagttacatgaaatagactattcgtcttacatgtatatgatcaaatctgacgtgaaacctaagactgatttaacaccgcaatttgaatactcagctctacagactgttgtgtatcacgagaagttgatcaactcgttgttcggtccaattttcaaagaaattaatgaacgcaagttggatgctatgcaaccacattttgtgttcaacacgagaatgacatcgagtgatttaaacgatcgagtgaagttcttaaatacggaagcggcttacgactttgttgagatagacatgtctaaattcgacaagtcggcaaatcgcttccatttacaactgcagctggagatttacaggttatttgggctagatgagtgggcggccttcctttgggaggtgtcgcacactcaaactactgtgagagatattcaaaatggtatgatggcgcatatttggtaccaacaaaagagtggagatgctgatacttataatgcaaattcagatagaacactgtgtgcactcttgtctgaattaccattggagaaagcagtcatggttacatatggaggagatgactcactgattgcgtttcctagaggaacgcagtttgttgatccgtgtccaaagttggctactaagtggaatttcgagtgcaagatttttaagtacgatgtcccaatgttttgtgggaagttcttgcttaagacgtcatcgtgttacgagttcgtgccagatccggtaaaagttctgacgaagttggggaaaaagagtataaaggatgtgcaacatttagccgagatctacatctcgctgaatgattccaatagagctcttgggaactacatggtggtatccaaactgtccgagtctgtttcagaccggtatttgtacaaaggtgattctgttcatgcgctttgtgcgctatggaagcatattaagagttttacagctctgtgtacattattccgagacgaaaacgataaggaattgaacccggctaaggttgattggaagaaggcacagagagctgtgtcaaacttttacgactggtaatatggaagacaagtcattggtcaccttgaagaagaagactttcgaagtctcaaaattctcaaatctaggggccattgaattgtttgtggacggtaggaggaagagaccgaagtattttcacagaagaagagaaactgtcctaaatcatgttggtgggaagaagagtgaacacaagttagacgtttttgaccaaagggattacaaaatgattaaatcttacgcgtttctaaagatagtaggtgtacaactagttgtaacatcacatctacctgcagatacgcctgggttcattcaaatcgatctgttggattcgagacttactgagaaaagaaagagaggaaagactattcagagattcaaagctcgagcttgcgataactgttcagttgcgcagtacaaggttgaatacagtatttccacacaggagaacgtacttgatgtctggaaggtgggttgtatttctgagggcgttccggtctgtgacggtacataccctttcagtatcgaagtgtcgctaatatgggttgctactgattcgactaggcgcctcaatgtggaagaactgaacagttcggattacattgaaggcgattttaccgatcaagaggttttcggtgagttcatgtctttgaaacaagtggagatgaagacgattgaggcgaagtacgatggtccttacagaccagctactactagacctaagtcattattgtcaagtgaagatgttaagagagcgtctaataagaaaaactcgtcttaatgcataaagaaatttattgtcaatatgacgtgtgtactcaagggttgtgtgaatgaagtcactgttcttggtcacgagacgtgtagtatcggtcatgctaacaaattgcgaaagcaagttgctgacatggttggtgtcacacgtaggtgtgcggaaaataattgtggatggtttgtctgtgttgttatcaatgattttacttttgatgtgtataattgttgtggccgtagtcaccttgaaaagtgtcgtaaacgtgttgaaacaagaaatcgagaaatttggaaacaaattcgacgaaatcaagctgaaaacatgtctgcgacagctaaaaagtctcataattcgaagacctctaagaagaaattcaaagaggacagagaatttgggacaccaaaaagatttttaagagatgatgttcctttcgggattgatcgtttgtttgctttttgattttattttatattgttatctgtttctgtgtatagactgtttgagattggcgcttggccgactcattgtcttaccataggggaacggactttgtttgtgttgttattttatttgtattttattaaaattctcaatgatctgaaaaggcctcgaggctaagagattattggggggtgagtaagtacttttaaagtgatgatggttacaaaggcaaaaggggtaaaacccctcgcctacgtaagcgttattacgccc

SEQ ID NO: 9 - DNA, 11003 bp, Artificial Sequence
  Synthetic plasmid pTRV2
  misc_feature (4824)..(4824): n is a, c, g, or t
ataaaacattgcacctatggtgttgccctggctggggtatgtcagtgatcgcagtagaatgtactaattgacaagttggagaatacggtagaacgtccttatccaacacagcctttatccctctccctgacgaggtttttgtcagtgtaatatttctttttgaactatccagcttagtaccgtacgggaaagtgactggtgtgcttatctttgaaatgttactttgggtttcggttctttaggttagtaagaaagcacttgtcttctcatacaaaggaaaacctgagacgtatcgcttacgaaagtagcaatgaaagaaaggtggtggttttaatcgctaccgcaaaaacgatggggtcgttttaattaacttctcctacgcaagcgtctaaacggacgttggggttttgctagtttctttagagaaaactagctaagtctttaatgttatcattagagatggcataaatataatacttgtgtctgctgataagatcattttaatttggacgattagacttgttgaactacaggttactgaatcacttgcgctaatcaacatgggagatatgtacgatgaatcatttgacaagtcgggcggtcctgctgacttgatggacgattcttgggtggaatcagtttcgtggaaagatctgttgaagaagttacacagcataaaatttgcactacagtctggtagagatgagatcactgggttactagcggcactgaatagacagtgtccttattcaccatatgagcagtttccagataagaaggtgtatttccttttagactcacgggctaacagtgctcttggtgtgattcagaacgcttcagcgttcaagagacgagctgatgagaagaatgcagtggcgggtgttacaaatattcctgcgaatccaaacacaacggttacgacgaaccaagggagtactactactaccaaggcgaacactggctcgactttggaagaagacttgtacacttattacaaattcgatgatgcctctacagctttccacaaatctctaacttcgttagagaacatggagttgaagagttattaccgaaggaactttgagaaagtattcgggattaagtttggtggagcagctgctagttcatctgcaccgcctccagcgagtggaggtccgatacgtcctaatccctagggatttaaggacgtgaactctgttgagatctctgtgaaattcagagggtgggtgataccatattcactgatgccattagcgacatctaaatagggctaattgtgactaatttgagggaatttcctttaccattgacgtcagtgtcgttggtagcatttgagtttcgcaatgcacgaattacttaggaagtggcttgacgacactaatgtgttattgttagataatggtttggtggtcaaggtacgtagtagagtcccacatattcgcacgtatgaagtaattggaaagttgtcagtttttgataattcactgggagatgatacgctgtttgagggaaaagtagagaacgtatttgtttttatgttcaggcggttcttgtgtgtcaacaaagatggacattgttactcaaggaagcacgatgagctttattattacggacgagtggacttagattctgtgagtaaggttaccgaattctctagaaggcctccatggggatccggtaccgagctcacgcgtctcgaggcccgggcatgtcccgaagacattaaactacggttctttaagtagatccgtgtctgaagttttaggttcaatttaaacctacgagattgacattctcgactgatcttgattgatcggtaagtcttttgtaatttaattttctttttgattttattttaaattgttatctgtttctgtgtatagactgtttgagatcggcgtttggccgactcattgtcttaccataggggaacggactttgtttgtgttgttattttatttgtattttattaaaattctcaacgatctgaaaaagcctcgcggctaagagattgttggggggtgagtaagtacttttaaagtgatgatggttacaaaggcaaaaggggtaaaacccctcgcctacgtaagcgttattacgcccgtctgtacttatatcagtacactgacgagtccctaaaggacgaaacgggagaacgctagccaccaccaccaccaccacgtgtgaattacaggtgaccagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgnctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggctgatgaatcccctaatgatttttatcaaaatcattaagttaaggtagatacacatcttgtcatatgatcaaatggtttcgccaaaaatcaataatcagacaacaaaatgtgcgaactcgatattttacacgactctctttaccaattctgccccgaattacacttaaaacgactcaacagcttaacgttggcttgccacgccttacttgactgtaaaactctcactcttaccgaacttggccgtaacctgccaaccaaagcgagaacaaaacataacatcaaacgaatcgaccgattgttaggtaatcgtcacctccacaaagagcgactcgctgtataccgttggcatgctagctttatctgttcgggcaatacgatgcccattgtacttgttgactggtctgatatccgtgagcaaaaacggcttatggtattgcgagcttcagtcgcactacacggtcgttctgttactctttatgagaaagcgttcccgctttcagagcaatgttcaaagaaagctcatgaccaatttctagccgaccttgcgagcattctaccgagtaacaccacaccgctcattgtcagtgatgctggctttaaagtgccatggtataaatccgttgagaagctgggttggtactggttaagtcgagtaagaggaaaagtacaatatgcagacctaggagcggaaaactggaaacctatcagcaacttacatgatatgtcatctagtcactcaaagactttaggctataagaggctgactaaaagcaatccaatctcatgccaaattctattgtataaatctcgctctaaaggccgaaaaaatcagcgctcgacacggactcattgtcaccacccgtcacctaaaatctactcagcgtcggcaaaggagccatggattctagcaactaacttacctgttgaaattcgaacacccaaacaacttgttaatatctattcgaagcgaatgcagattgaagaaaccttccgagacttgaaaagtcctgcctacggactaggcctacgccatagccgaacgagcagctcagagcgttttgatatcatgctgctaatcgccctgatgcttcaactaacatgttggcttgcgggcgttcatgctcagaaacaaggttgggacaagcacttccaggctaacacagtcagaaatcgaaacgtactctcaacagttcgcttaggcatggaagttttgcggcattctggctacacaataacaagggaagactcactcgtggctgcaaccctgcttactcaaaatctattcacacatggttacgttttggggaaattatgaggggatctctcagcgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcctaggccaccatgttgggcccggcgcgccaagcttgcatgcctgcaggtcaacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatggtcaacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagg

SEQ ID NO: 10 - DNA, 10644 bp, Artificial Sequence
  Synthetic TuMV-GFP construct
aaaaaatataaaaactcaacataacatacacaaaacgattaaagcaaacacaaatctttcaaagcattcaagcaatcaaagattctcaaatctttcatcgttatcaaagcaatcaccaacagcaaaccaaatggcagcagttacattcgcatcagctatcaccaacgccatcaccagcaaaccagcactcaccggaatggtgcagtttgggagtttcccaccaatgccattgcgatccaccaccgtcaccacagtcgccacttcagtggcgcaacctaaactgtacacagtgcagtttggaagccttgacccagtagtcgtcaagagtggagcagggtcccttgctaaggcaacacgccagcagcctaacgttgaaatagacgttagcctcagtgaagccgcagctctggaggttgcgaaacctagatcgaatgccgtgttgaggatgcacgaggaagcaaacaaggagagagcactctttttggactgggaggctagtttgaagagaagctcgtatggaattgctgaggacgagaaggttgtaatgacaactcatggcgtcagcaagatagtgcccagaagttcaagggcaatgaagctaaagcgcgcaagggagaggcgtagagcgcagcaaccaattatattaaagtgggagcccaaattgagcgggatctcaatcggaggagggctctctgcgagcgtaatcgaagcagaagaggttcgcacaaagtggccgcttcataagacaccgtcaatgaagaagaggacggtgcacagaatatgcaagatgaacgaccaaggagttgacatgttgacacgatccctggttaagattttcaagactaagagtgccaacattgaatacatcggaaagaagtcgattaaggtcgatttcatcagaaaagaacgaacgaaattcgcaagaatccaagtagcacacttactcgggaagagagcacagcgcgacttgttaactggaatggaagaaaaccattttattgacattctcagtaagtactcaggtaacaaaacaaccataaatcctggagtagtttgcgcaggttggagtggcatagtcgttggaaatggaattctaacccagaaacgaagcagaagtccatcagaggcctttgtaattagaggtgagcacgaaggcaagttgtacgatgccaggatcaaagtcacgaggacaatgagtcacaagattgtgcactttagtgccatggcaagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccttggccaacacttgtcactactttctcttatggtgttcaatgcttttcaagatacccagatcatatgaagcggcacgacttcttcaagagcgccatgcctgagggatacgtgcaggagaggaccatctctttcaaggacgacgggaactacaagacacgtgctgaagtcaagtttgagggagacaccctcgtcaacaggatcgagcttaagggaatcgatttcaaggaggacggaaacatcctcggccacaagttggaatacaactacaactcccacaacgtatacatcacggcagacaaacaaaagaatggaatcaaagctaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaaggatccgttgaggcttgtgtctatcaccaggcaggtgcagcgggagccaacttctggaaaggcttcgacagatgctttctcgcataccgtagtgacaatcgcgagcatacatgctattcagggctagatgtcactgagtgcggcgaggtggcagcactgatgtgtttggctatgttcccatgcggaaagataacctgccctgactgtgtaacagatagtgagctatcccaaggacaagcaagcggaccatctatgaagcacaggttgacacagctacgcgatgtcatcaagtcaagctacccacgcttcaagcatgcagtgcagatactagataggtatgagcaatcactgagcagtgcaaacgagaactaccaagatttcgcagaaatccagagcataagcgatggagttgaaaaagctgcattcccacacgtcaacaagctaaacgcaatattgatcaaaggggccacagtaacaggagaggaattctcgcaggctacgaagcacttgctcgagatagcacgatacctgaagaacagaaccgagaacattgagaagggttcactgaagtcctttcgcaacaagatttcccagaaagcgcacatcaacccaacactaatgtgtgacaaccagctcgatagaaatggaaatttcatatggggtgagagaggataccatgcaaaacgattcttcagcaactactttgaaataatcgatccaaagaaaggctacacccaatacgagacaagagcggtaccaaatgggtcacggaaacttgcaatcggcaaactaatagtcccaacgaacttcgaagttttaagggaacagatgaaaggcgaaccggtagaaccatacccagtaacagtcgagtgtgtgagcaagttacagggtgacttcgtccatgcatgttgttgtgtcacaacagaatcaggcgacccagtcttgtctgagatcaaaatgccaaccaaacaccatctagtgattggtaacagcggtgatccaaagtacatagatctccctgagatcgaggagaataaaatgtacatagcgaaagaaggttattgttacatcaatatcttcctagccatgttggtaaatgtcaaggagtcgcaggcaaaggagttcacgaaagttgttagggacaaactagttggcgaacttggcaagtggcccactctgttagatgtagcaaccgcttgttatttcctgaaagtattttacccagacgttgctaacgccgaattgccacgcatgctagtggaccataagacaaagataattcatgtcgttgattcatatgggtcactgtcaactggatatcatgtccttaagacaaacactgtggaacaactcatcaaattcacgagatgtaatttggagtcaagcttgaaacactaccgcgttggaggaacagaatgggaggacactcatggatccagcaacatagataatccacagtggtgcatcaagaggctcataaaaggagtctacaaaccaaagcaactgaaagaagacatgttggcaaaccctttcttaccactatatgctctactgtcaccaggtgtcatcctggcattttacaatagtggctctctagagtacttgatgaaccattacatcagggtggacagcaacgtcgccgttttgttggtcgttttgaaatctctagcgaagaaggtgtcaactagtcagagtgtgttagcccagcttcaaatcattgaacgaagtctaccagaactcatcgaagcaaaggctaatgttaatgggccagatgacgcagccactcgcgcgtgtaacagattcatgggcatgcttctgcatatggcagaaccaaactgggagcttgcggatggtggatacacaattctgagggatcatagcatctccattttggaaaaaagttatctacaaatcttggacgaagcatggaacgagttaagttggtcggagcgctgtgctataagatactactcgtcaaagcaagcaatctttacacagaaagatttgccaatgaaaagcgaagccgatttaggcggcagatacagcgtgtcagtcatgtcatcttacgaacggagtaagcaatgtatgaaaagcgtgcactctagtataggtaatagattacgtagtagtatgtcttggactagtagcaaggtgtcgaatagtgtgtgtaggactattaactatttagtaccagatgtgttcaagtttatgaatgtactcgtttgtatcagcttactaatcaagatgactgccgaggcgaatcacatcgtcaccacgcaaagaaggctcaaactagatgtcgaggagacagagcgcaggaaaatagaatgggagcttgcattccaccatgccattctgacgcagagtgcaggtcaacacccaacgatagacgagttcagagcgtacatcgccgacaaggcaccacatctaagtgagcatatcgagcctgaagaaaaggcggtggttcatcaagcgaagagacaatccgagcaagaactcgagcgtataatagcatttgttgcattggtgctcatgatgttcgatgcagaacgaagcgactgtgtcacaaagattctcaacaagcttaagggactagtcgccactgtggaacctacagtctaccatcagactctcaatgatatagaggatgacttgagtgagaggaacctcttcgtcgattttgagcttagcagcgatggagatatgctccaacagcttccagccgaaaagacatttgcctcatggtggagtcatcaactaagcagaggattcacaatcccacactacaggacagaagggaagttcatgactttcaccagagcaactgccacggaagtcgcgggtaaaatagcacacgagagtgacaaagacatattactaatgggagcagtaggatcaggtaagtcaactggcttgccatatcatctctccagaaaagggaacgtattactccttgagccgactcggccacttgcagaaaacgtacacaagcagttgtcgcaggcaccgttccatcagaacacaactcttaggatgcgcggactaacagcattcgggtcggcaccaatctcagtgatgaccagtggttttgcactcaattactttgcaaacaacagaatgcgaattgaagaatttgactttgtcatatttgatgaatgtcacgttcatgacgccaatgcaatggcgatgagatgtttgctacatgagtgtgactattctggcaaaattatcaaagtttcagccacaccaccaggtcgagaagttgagttctccactcaataccccgtgtcgataagcacagaagacacactatcgtttcaggattttgtgaacgcacagggtagtggaagcaattgtgatgtgatttcaaaaggagacaatatcctcgtgtatgtagcaagctacaatgaggtagacgcgctttcaaaacttctaattgaaagagacttcaaagtcacgaaggttgatggaagaacgatgaaagttggaaacatcgagatcaccacaagtggaacacctagtaagaagcacttcatagttgcaaccaacatcatagagaacggtgttactctagacatcgatgtggttgctgattttggaacgaaggtactcccatatcttgatacagacagcagaatgctgagcacaactaagacaagcatcaattatggggaacgtatccaaaggctaggaagagtcggaaggcacaagccaggtcacgctctgcgaataggtcacacagagaaggggttgagcgaagttccaagttgtattgcaacagaagcagctttaaagtgcttcacttatgggcttccagtgatcaccaacaacgtctcgacaagtattcttggtaatgtaacggtaaagcaggcacgaacaatgtctgtatttgagataacaccgttctacacaagccaagtggtgagatatgatggctccatgcatccacaggtgcacgcactcttaaagagattcaaactcagagactctgagattgttttgaataaattagccatacctcaccgaggagtgaacgcttggctcacagctagtgagtatgcacgacttggcgcgaatgttgaagataggcgtgacgttcgaattccttttatgtgtcgcgacatcccagaaaaacttcatctagacatgtgggatgtgattgttaaattcaaaggtgatgcaggttttggtcggctttcaagcgccagtgcgagcaaggtagcttatactctacagacggacgtcaactccatacagcgaacagtcactatcatagatacactaatcgctgaggagagaaggaagcaggaatacttcaagacggtaacctccaactgtgtctcttcttcgaacttctcactgcagagcataacaaatgcgataaaatctcgtatgatgaaagatcacacgtgcgagaacatatcagtgcttgaaggagcgaagtcacagttactcgagtttagaaacctgaatgctgatcactcatttgctacaaaaaccgatggaatatctcggcatttcatgagtgagtatggagctcttgaggcagttcaccatcaaaacaccagcgacatgagcaaattcctcaagcttaagggcaaatggaataaaacgctaatcacgcgagatgtgctggtactttgtggagttcttggaggtggattgtggatggttattcagcacctgcggtcaaagatgtccgaacccgtaacccatgaagcgaaaggtaagaggcaaaggcagaaactaaaatttcgcaatgcccgagacaacaaaatgggtagagaagtgtacggagatgatgataccatagagcatttcttcggtgatgcctacacaaagaaagggaagagcaagggtaggacacgtggtatcggacacaaaaacaggaagttcatcaacatgtatgggtttgatcctgaagatttctctgcagttcgtttcgtggatccactcacaggagcgacgttggacgacaacccgctcacagacatcacccttgtgcaagagcacttcggcaacataagaatggacttactcggggaggatgagctggactcaaatgaaatacgtgtgaataagactattcaagcctactacatgaacaataaaacaggcaaggctttgaaggtggatctgacaccacacatacctctcaaggtgtgtgatcttcacgcaaccattgctggattcccagagcgagaaaacgagctgaggcagactggaaaggctcagcccatcaacatagacgaagtgccaagagctaacaacgaactcgtcccagtggaccacgagagtaactccatgttcagagggttgcgtgactacaacccaatatcaaacaacatttgtcatctcacaaatgtttcagatggagcatcaaactcgttatatggagtcggtttcggaccactcatattaacgaaccgacacctctttgagcggaataacggtgaactcgtaataaaatcacgacatggtgagttcgtgattaaaaacacaactcagctacacttgctaccgattccagacagagatcttctgctaatccggttaccaaaggacgtcccaccctttccacagaaattgggtttcaggcaacctgagaaaggtgaacgaatttgcatggtggggtccaatttccaaaccaagagcataacgagtatagtctctgagactagtacaataatgccagtggagaacagtcagttttggaaacactggattagcactaaagacggccaatgcggaagtccaatggtgagcacgaaagacgggaaaatactcggattacacagcctagcgaacttccagaactccatcaattactttgctgctttcccagatgattttgccgagaagtatcttcataccattgaagcacacgagtgggtcaagcactggaagtataacactagcgccatcagttggggctctttgaatatacaagcatcgcaaccgtccggcttgttcaaagtaagcaagctaatctcagacctcgacagcacggcagtctacgcacaaacccagcagaatcggtggatgttcgagcagctcaacgggaacctaaaagcgatagcacactgccctagccagcttgtgacaaagcacacagttaaaggaaaatgtcagatgtttgacttgtatctcaagttgcatgatgaagcacgagagtatttccaaccgatgctgggccagtatcaaaagagcaaactcaatcgagaagcatatgcaaaggatcttctgaaatatgcaacgccaatcgaagcaggaaacatcgactgtgatctgtttgaaaagacagttgaaatagtcgtatcagatctgcgaggttatggtttcgaaacatgcaattatgtcactgatgagaatgacatattcgaagctcttaacatgaaatccgcagttggagcgttgtataaaggaaagaagaaggattacttcgctgagttcacacccgagatgaaagaagaaatactgaaacaaagttgtgaacggctcttcctaggaaagatgggagtgtggaacggctcgctgaaggcagagttgcgaccactagaaaaagtggaagcaaacaaaacacggacgtttactgccgcaccactagacacactgttgggtggaaaagtttgcgtggatgatttcaacaaccagttctatgatcacaaccttagagctccttggagcgttggcatgacaaagttttattgtggttgggatcgcttgttggagtcgttgccagatggttgggtgtattgcgatgctgatggctcacagttcgacagctcgctatcgccatacttgatcaacgcagtactcaacatccgcttaggattcatggaagagtgggacataggggaggtaatgctgagaaatttgtacaccgaaatcgtgtatacccctatttctacaccagatggtacactcgtcaagaagttcaaaggaaacaatagcggacagccatcgactgttgtggacaacacgctcatggtcatattggcagtcaactattcactcaagaaaagcggaattccaagtgagttgcgcgacagcatcatcagattcttcgtcaacggagatgatttactgctaagcgtacacccagagtatgagtatattcttgacactatggcagacaactttcgtgaactgggcctgaagtatactttcgactcaagaaccagggaaaaaggagacctctggtttatgtcgcaccaggggcacaaaagagagggaatctggattcccaagctcgagccagagcgaatagtatcgattctagaatgggatcggtcgaaagagccatgccatcgactagaggcaatctgcgcagcgatgattgagtcgtggggatacgacaagttaactcacgagatacgcaagttctacgcgtggatgattgaacaagctccatttagctccctagcacaagaagggaaagctccttacatagcggaaacagcgctgaggaagctctaccttgataaggaaccagctcaagaggatctcacccattatttgcaagcaatctttgaggattatgaagatggtgctgaggcttgtgtttatcaccaggcaggtgaaacgcttgatgcaggtttgacagacgagcaaaagcaggcagagaaggagaagaaggagagagagaaggcagaaaaggaacgagagaggcaaaagcagttggcactcaagaaaggcaaggatgttgcacaagaagagggaaaacgcgacaaggaagtaaacgctggaacctctggaactttcagtgtacccagactcaagagtctgacaagcaagatgcgcgtgccaagatacgagaaaagagtggctctaaacctcgatcatctaatcctatacacgccggagcagacggatctatccaacacacgttcaacgcgaaagcagtttgacacatggtttgaaggtgtaatggctgattacgaactgacggaggacaaaatgcaaatcattctcaatggtttaatggtctggtgcattgagaacggaacctccccgaacataaacggaatgtgggtgatgatggacggcgacgatcaggtggaattcccgatcaaaccgctcattgaccacgccaaacccacatttaggcagataatggcccatttcagtgacgtagctgaagcgtacattgaaaagcgtaaccaagaccgaccatacatgccacgatatggtcttcagcgcaatttaaccgacatgagcttagctcgatacgcatttgatttctatgaaatgacttctaggactccaatacgtgcgagagaggcacacatccagatgaaagcagcagcactgcgtggcgcaaataataatttgttcggcttggatggaaacgttggtacaacggtagagaacacggagaggcatacgaccgaggacgttaatcggaacatgcataacttactgggcgttcaggggttgtgaagttgtatgctggtagactataagtatttaagtttactcgttagtattctcgcttatgggaaatatgtaagtttgttaaagcagccagtgtgactttgtcatgtgtgttgttgttactttctgtattttcgccgaacattttattggtgttagcgcatgtagtgaggatcgtcctcgattgccttaacatttgataggatgcaagggacaaaaaaaaaaaaaaaaaaaaaaaaaaaaaaaaaaaaaaaaaaaaaaaaaaaaaa

SEQ ID NO: 11 - DNA, 2716 bp, Artificial Sequence
  Synthetic plasmid pENTR-AtU6LbuDRBsaI
ctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctccgcggccgcccccttcacctgatcaaaagtcccacatcgatcaggtgatatatagcagcttagtttatataatgatagagtcgacatagcgattggatttagaccaccccaaaaatgaaggggactaaaacaagagaccttttttttttgagaccaagggtgggcgcgccgacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtt

SEQ ID NO: 12 - DNA, 2714 bp, Artificial Sequence
  Synthetic plasmid pENTR-AtU6LbaDRBsaI
ctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctccgcggccgcccccttcacctgatcaaaagtcccacatcgatcaggtgatatatagcagcttagtttatataatgatagagtcgacatagcgattggctggagaagatagcccaagaaagagggcaataacagagaccttttttttttgagaccaagggtgggcgcgccgacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtt

SEQ ID NO: 13 - DNA, 3034 bp, Artificial Sequence
  Synthetic plasmid pCR8PEBVLbuDRBsaI
agggcgaattcgacccagctttcttgtacaaagttggcattataaaaaataattgctcatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgcctcctctagaccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataaccctcgagccacccatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctccgaattcgccctaattcgagcatcttgttctggggtttcacactatctttagagaaagtgttaagttaattaagttatcttaattaagagcataattatactgatttgtctctcgttgatagagtctatcattctgttactaaaaatttgacaactcggtttgctgacctactggttactgtatcacttacccgagttaacgagaacaagagaccttttttttttgagacc

SEQ ID NO: 14 - DNA, 75 bp, Artificial Sequence
  Synthetic AtU6 promoter
tgatcaaaagtcccacatcgatcaggtgatatatagcagcttagtttatataatgatagagtcgacatagcgatt

SEQ ID NO: 15 - DNA, 192 bp, Artificial Sequence
  Synthetic PEBV promoter
aattcgagcatcttgttctggggtttcacactatctttagagaaagtgttaagttaattaagttatcttaattaagagcataattatactgatttgtctctcgttgatagagtctatcattctgttactaaaaatttgacaactcggtttgctgacctactggttactgtatcacttacccgagttaacgag

SEQ ID NO: 16 - DNA, 60 bp, Artificial Sequence
  Synthetic Lbu crRNA cloning backbone
gatttagaccaccccaaaaatgaaggggactaaaacaagagaccttttttttttgagacc

SEQ ID NO: 17 - DNA, 57 bp, Artificial Sequence
  Synthetic Lba crRNA cloning backbone
gctggagaagatagcccaagaaagagggcaataacggtctcgtaactttttgagacc

SEQ ID NO: 18 - DNA, 28 bp, Artificial Sequence
  Synthetic guide sequence
gtttctgcctttgcctcttacctttcgc

SEQ ID NO: 19 - DNA, 28 bp, Artificial Sequence
  Synthetic guide sequence
ctgggaaatcttgttgcgaaaggacttc

SEQ ID NO: 20 - DNA, 28 bp, Artificial Sequence
  Synthetic guide sequence
ttgtgtttgctttaatcgttttgtgtat

SEQ ID NO: 21 - DNA, 164 bp, Artificial Sequence
  Synthetic guide sequence
taacgtttctgcctttgcctcttacctttcgcgctggagaagatagcccaagaaagagggcaataacctgggaaatcttgttgcgaaaggacttcgctggagaagatagcccaagaaagagggcaataacttgtgtttgctttaatcgttttgtgtattttttt

SEQ ID NO: 22 - DNA, 168 bp, Artificial Sequence
  Synthetic guide sequence
aacagtttctgcctttgcctcttacctttcgcgatttagaccaccccaaaaatgaaggggactaaaacactgggaaatcttgttgcgaaaggacttcgatttagaccaccccaaaaatgaaggggactaaaacattgtgtttgctttaatcgttttgtgtattttttt

SEQ ID NO: 23 - DNA, 28 bp, Artificial Sequence
  Synthetic guide sequence
cctttcggtacttcgtccacaaacacaa

SEQ ID NO: 24 - DNA, 28 bp, Artificial Sequence
  Synethetic guide sequence
gtccaatttggggcattttattgaacaa

SEQ ID NO: 25 - DNA, 28 bp, Artificial Sequence
  Synthetic guide sequence
tcgaaagctcgtcagggtttatgaagtt

SEQ ID NO: 26 - DNA, 28 bp, Artificial Sequence
  Synthetic guide sequence
gaagtaactcgtaatcctgtacaatagc

SEQ ID NO: 27 - DNA, 169 bp, Artificial Sequence
  Synthetic guide sequence
aacagtccaatttggggcattttattgaacaagatttagaccaccccaaaaatgaaggggactaaaacatcgaaagctcgtcagggtttatgaagttgatttagaccaccccaaaaatgaaggggactaaaacagaagtaactcgtaatcctgtacaatagcttttttt

SEQ ID NO: 28 - DNA, 169 bp, Artificial Sequence
  Synthetic guide sequence
aacaccttttgactcaatatgttccacaatcggatttagaccaccccaaaaatgaaggggactaaaacaacagacatgtcagcgtacacactgagcagatttagaccaccccaaaaatgaaggggactaaaacagggaaaagcttcgctagttccttcattgttttttt

SEQ ID NO: 29 - DNA, 28 bp, Artificial Sequence
  Synthetic nontargeting control guide
cctttcggtacttcgtccacaaacacaa

SEQ ID NO: 30 - DNA, 166 bp, Artificial Sequence
  Synthetic guide sequence
aacagtccgctttggggcattttattgaacaagatttagaccaccccaaaaatgaaggggactaaaacatcgagcgctcgtcagggtttatgaagttgatttagaccaccccaaaaatgaaggggactaaaacagaagcgactcgtaatcctgtacaatagctttt

SEQ ID NO: 31 - DNA, 166 bp, Artificial Sequence
  Synthetic guide sequence
aacagtccaattttaggcattttattgaacaagatttagaccaccccaaaaatgaaggggactaaaacatcgaaagctattcagggtttatgaagttgatttagaccaccccaaaaatgaaggggactaaaacagaagtaacttataatcctgtacaatagctttt

SEQ ID NO: 32 - DNA, 166 bp, Artificial Sequence
  Synthetic guide sequence
aacagtccaatttggggcattttagcgaacaagatttagaccaccccaaaaatgaaggggactaaaacatcgaagctcgtcagggttttcggaagttgatttagaccaccccaaaaatgaaggggactaaaacagaagtaactcgtaatcctgttgaatagctttt

SEQ ID NO: 33 - DNA, 117 bp, Artificial Sequence
  Synthetic nontargeting guide control
aacacctttcggtacttcgtccacaaacacaacgggaaccattcaaaacagcatagcaagttaaaataaggctagtccgttatcaacttgaaaaagtggcaccgagtcggtgctttt

SEQ ID NO: 34 - DNA, 117 bp, Artificial Sequence
  Synthetic guide sequence
aacagaagtaactcgtaatcctgtacaatagccgggaaccattcaaaacagcatagcaagttaaaataaggctagtccgttatcaacttgaaaaagtggcaccgagtcggtgctttt

SEQ ID NO: 35 - DNA, 117 bp, Artificial Sequence
  Synthetic guide sequence
aacagtacttagtggaattgcagaagatttggcgggaaccattcaaaacagcatagcaagttaaaataaggctagtccgttatcaacttgaaaaagtggcaccgagtcggtgctttt

SEQ ID NO: 36 - DNA, 165 bp, Artificial Sequence
  Synthetic guide sequence
aacataagctgaactaccttggactctcaagtgatttagaccaccccaaaaatgaaggggactaaaacattgaaagttcgtcagggtttataaagttgatttagacaccccaaaaatgaaggggactaaaacacaagtaactcataatcctgtacaatagctttt

SEQ ID NO: 37 - DNA, 28 bp, Artificial Sequence
  Synthetic nontargeting control guide
cctttcggtacttcgtccacaaacacaa

SEQ ID NO: 38 - DNA, 24 bp, Artificial Sequence
  Forward primer
ggactggatgagaaagcaaggtgt

SEQ ID NO: 39 - DNA, 24 bp, Artificial Sequence
  Reverse primer
atcgaaagctcgtcagggtttatg

SEQ ID NO: 40 - DNA, 21 bp, Artificial Sequence
  Forward primer
agctttacctcccaagtcatc

SEQ ID NO: 41 - DNA, 20 bp, Artificial Sequence
  Reverse primer
agaacgcctgtcaatcttgg

SEQ ID NO: 42 - DNA, 23 bp, Artificial Sequence
  Forward primer
gaaggagaagaaggagagagaga

SEQ ID NO: 43 - DNA, 21 bp, Artificial Sequence
  Reverse primer
cagaggttccagcgtttactt

SEQ ID NO: 44 - DNA, 22 bp, Artificial Sequence
  Forward primer
tgaggacgagaaggttgtaatg

SEQ ID NO: 45 - DNA, 18 bp, Artificial Sequence
  Reverse primer
attggttgctgcgctcta

SEQ ID NO: 46 - DNA, 22 bp, Artificial Sequence
  Forward primer
ccgaacaaggaacaagtgaaag

SEQ ID NO: 47 - DNA, 22 bp, Artificial Sequence
  Reverse primer
gtcctcatcttcaatgccagta

SEQ ID NO: 48 - DNA, 21 bp, Artificial Sequence
  Forward primer
cgcctcagagctgtaaacata

SEQ ID NO: 49 - DNA, 20 bp, Artificial Sequence
  Reverse primer
ctccgaaccagccgaataaa

SEQ ID NO: 50 - DNA, 25 bp, Artificial Sequence
  Forward primer
ggtcacaaaccgatattgctggagg

SEQ ID NO: 51 - DNA, 28 bp, Artificial Sequence
  Reverse primer
acagacatgtcagcgtacacactgagca

SEQ ID NO: 52 - DNA, 27 bp, Artificial Sequence
  Forward primer
gctgaaaatatttaggcagttgaactc

SEQ ID NO: 53 - DNA, 23 bp, Artificial Sequence
  Reverse primer
ggaaccctttaagttcgtcagct

SEQ ID NO: 54 - DNA, 24 bp, Artificial Sequence
  Forward primer
gttcgggattatgcgaaagtgacc

SEQ ID NO: 55 - DNA, 23 bp, Artificial Sequence
  Reverse primer
acggaaaacctccagttcctcgc

SEQ ID NO: 56 - DNA, 34 bp, Artificial Sequence
  Reverse primer
gactagcgaattcaattcgagcatcttgttctgg

SEQ ID NO: 57 - DNA, 29 bp, Artificial Sequence
  Forward primer
cagtacaacgcgtctgggtcgaattcgcc

SEQ ID NO: 58 - DNA, 85 bp, Artificial Sequence
  Synthetic nanostring probe
cgtgtataggattagatgatcgaggtttagagccactcttttctcgtatccaacagccactttttttccaaattttgcaagagcc

SEQ ID NO: 59 - DNA, 63 bp, Artificial Sequence
  Synthetic nanostring probe
cgaaagccatgacctccgatcactccgcgttgaacgtgtgttggatagatccgtctgctccgg

SEQ ID NO: 60 - DNA, 85 bp, Artificial Sequence
  Synthetic nanostring probe
cgaaaggacttcagtgaacccttctcaatgttctcggttctgttcttcagcaccgtgtggacggcaactcagagataacgcatat

SEQ ID NO: 61 - DNA, 75 bp, Artificial Sequence
  Synthetic nanostring probe
cgaaagccatgacctccgatcactctgtcacacattagtgttgggttgatgtgcgctttctgggaaatcttgttg

SEQ ID NO: 62 - DNA, 85 bp, Artificial Sequence
  Synthetic nanostring probe
cgcagttacgcacataggtgtccagcttgtttaacagcttattttcgatacatcctcttcttttcttggtgttgagaagatgctc

SEQ ID NO: 63 - DNA, 75 bp, Artificial Sequence
  Synthetic nanostring probe
cgaaagccatgacctccgatcactcgaagtctgaagtggctatctccccgtcttgcaaataatagttgtacttgc

SEQ ID NO: 64 - DNA, 85 bp, Artificial Sequence
  Synthetic nanostring probe
ttgggtaagccccaaagaatatgtgcaacccagtctcgtaccaatctccacaaagacgcctatcttccagtttgatcgggaaact

SEQ ID NO: 65 - DNA, 75 bp, Artificial Sequence
  Synthetic nanostring probe
cgaaagccatgacctccgatcactcccactgcaaccgatcgttaatccctagttctccaaacaggttctgcatat

SEQ ID NO: 66 - DNA, 85 bp, Artificial Sequence
  Synthetic nanostring probe
cagggacggttccaatgccaccaatcttgtaaacatcctgaagtggaagaccaatttggttttactcccctcgattatgcggagt

SEQ ID NO: 67 - DNA, 60 bp, Artificial Sequence
  Synthetic nanostring probe
cgaaagccatgacctccgatcactcaccaggcttgagcacaccagtctccacacgaccaa

SEQ ID NO: 68 - DNA, 79 bp, Artificial Sequence
  Synthetic nanostring probe
tcccatcaggcctaatcaaggtattgactttctttgtctggatgcgaacctaactcctcgctacattcctattgttttc

SEQ ID NO: 69 - DNA, 75 bp, Artificial Sequence
  Synthetic nanostring probe
cgaaagccatgacctccgatcactcaacgtccaatgcatcgtagtcaggagtcaacctcacatatgctttcttcg

SEQ ID NO: 70 - DNA, 85 bp, Artificial Sequence
  Synthetic nanostring probe
cttgccaagtgcagcacaaccctcaacagctaataaacgtacagaatcctcagataaggttgttattgtggaggatgttactaca

SEQ ID NO: 71 - DNA, 75 bp, Artificial Sequence
  Synthetic nanostring probe
cgaaagccatgacctccgatcactcttgacaatgacaggcaggatgtgtgcaacacaatcctgtggctccaacag

SEQ ID NO: 72 - DNA, 28 bp, Artificial Sequence
  Synthetic guide sequence
taacgaatctggagctgaattatccatg

SEQ ID NO: 73 - DNA, 28 bp, Artificial Sequence
  Synthetic guide sequence
ctacaagttccgagacgtttcggctcta

SEQ ID NO: 74 - DNA, 28 bp, Artificial Sequence
  Synthetic guide sequence
aggagctgcattttgttagcaaaagcaa

SEQ ID NO: 75 - DNA, 166 bp, Artificial Sequence
  Synthetic guide sequence
aacataacgaatctggagctgaattatccatggatttagaccaccccaaaaatgaaggggactaaaacactacaagttccgagacgtttcggctctagatttagaccaccccaaaaatgaaggggactaaaacaaggagctgcattttgttagcaaaagcaatttt

SEQ ID NO: 76 - DNA, 168 bp, Artificial Sequence; identical to SEQ ID NO: 22
  Synthetic nontargeting control guide